(19) United States
(12) Patent Application Publication
(10) Pub. No.: US 2025/0064948 A1
(43) Pub. Date: Feb. 27, 2025

(54) GCPII INHIBITION FOR THE TREATMENT OF SARCOPENIA AND AGING

(71) Applicant: The Johns Hopkins University, Baltimore, MD (US)

(72) Inventors: Barbara SLUSHER, Kingsville, MD (US); Carolyn TALLON, Baltimore, MD (US)

(21) Appl. No.: 18/729,564

(22) PCT Filed: Feb. 14, 2023
(86) PCT No.: PCT/US2023/062528

§ 371 Date: Jul. 17, 2024

Related U.S. Application Data
(60) Provisional application No. 63/309,846, filed on Feb. 14, 2022.

(51) Int. Cl. A61K 47/59; A61K 31/662; A61P 21/00

(52) U.S. Cl. CPC A61K 47/595; A61K 31/662; A61P 21/00

(57) ABSTRACT

Methods for treating age-related sarcopenia and/or enhancing longevity by administering one or more GCPII inhibitors, wherein the one or more GCPII inhibitors are selected from a phosphonate-based GCPII inhibitor, a phosphinate-based GCPII inhibitor, a phosphoramidate-based GCPII inhibitor, a thiol-based inhibitor, a hydroxamate-based inhibitor, and a urea-glutamate based GCPII inhibitor, including one or more of 2-PMPA and prodrugs thereof, L-DOPA, D-DOPA, caffeic acid, and prodrugs thereof, a hydroxamate-based prodrug, and a dendrimer 2-PMPA conjugate are disclosed.

### FEDERALLY SPONSORED RESEARCH OR DEVELOPMENT

This invention was made with government support under grant AG068130 awarded by the National Institutes of Health. The government has certain rights in the invention.

### BACKGROUND

Sarcopenia is the progressive loss of muscle mass and function and is a natural part of the aging process. Sarcopenia can contribute to frailty and increase the likelihood of falls and fractures in older adults. With an aging population in the United States, sarcopenia is predicted to have significant negative economic ramifications. Thus, being able to delay the onset and slow the progression of sarcopenia would be beneficial in terms of improved health and economics.

### SUMMARY

In some aspects, the presently disclosed subject matter provides a method for treating age-related sarcopenia and/or enhancing longevity, the method comprising administering to a subject in need of treatment thereof one or more GCPII inhibitors.

In certain aspects, the one or more GCPII inhibitors are selected from a phosphonate-based GCPII inhibitor, a phosphinate-based GCPII inhibitor, a phosphoramidate-based GCPII inhibitor, a thiol-based inhibitor, a hydroxamate-based inhibitor, and a urea-glutamate based GCPII inhibitor.

In particular aspects, the phosphonate-based GCPII inhibitor is selected form 2-(phosphonomethyl) pentanedioic acid (2-PMPA), GPI-5232, and VA-033.

In particular aspects, the phosphinate-based GCPII inhibitors are selected from 2-[[methylhydroxyphosphinyl]methyl]pentanedioic acid, 2-[[ethylhydroxyphosphinyl]methyl]pentanedioic acid, 2-[[propylhydroxyphosphinyl]methyl]pentanedioic acid, 2-[[butylhydroxyphosphinyl]methyl]pentanedioic acid, 2-[[cyclohexylhydroxyphosphinyl]methyl]pentanedioic acid, 2-[[phenylhydroxyphosphinyl]methyl]pentanedioic acid, 2-[[(phenylmethyl)hydroxyphosphinyl]methyl]pentanedioic acid, 2-[[((2-phenylethyl)methyl)hydroxyphosphinyl]methyl]pentanedioic acid, 2-[[((3-phenylpropyl)methyl)hydroxyphosphinyl]methyl]pentanedioic acid, 2-[[((3-phenylbutyl)methyl)hydroxyphosphinyl]methyl]pentanedioic acid 2-[[((2-phenylbutyl)methyl)hydroxyphosphinyl]methyl]pentanedioic acid, 2-[[(4-phenylbutyl)hydroxyphosphinyl]methyl]pentanedioic acid, 2-[[(aminomethyl)hydroxyphosphinyl]methyl]pentanedioic acid; 7-(L-2-amino-2-carboxyethylthio)-2-(2,2-dimethylcyclopropanecarboxamide)-2-heptenoic acid, 2-(phosphonomethyl)pentanedioic acid, N-[methylhydroxyphosphinyl]glutamic acid; N-[ethylhydroxyphosphinyl]glutamic acid, N-[propylhydroxyphosphinyl]glutamic acid; N-[butylhydroxyphosphinyl]glutamic acid, N-[phenylhydroxyphosphinyl]glutamic acid; aN-[(phenylmethyl)hydroxyphosphinyl]glutamic acid; (S)-2-((N—((S)-1,2-nd dicarboxyethyl)sulfamoyl)amino)pentanedioic acid.

In certain aspects, the thiol-based GCPII inhibitor is selected from 2-(3-mercaptopropyl)pentanedioic acid (2-MPPA), 3-(2-mercaptoethyl)biphenyl-2,3-dicarboxylic acid (E2072) and GPI-5693.

In certain aspects, the hydroxamate-based GCPII inhibitor is selected from 2-(2-(hydroxyamino)-2-oxoethyl)pentanedioic acid (JHU 241) and 4-carboxy-alpha-[3-(hydroxyamino)-3-oxopropyl]-benzenepropanoic acid.

In certain aspects, the urea-glutamate based GCPII inhibitor is selected from N—[N—[(S)]-1,3-dicarboxypropyl]carbamoyl]-L-leucine (ZJ-43), MIP-1555, MIP-1519, MIP-1545, MIP-1427, MIP-1428, MIP-1379, MIP-1072, MIP-1095, MIP-1558, MIP-1405, MIP-1404, PSMA I&T, PSMA-617, PSMA-11, DCIBzL, ¹⁸F-DCFPyl, ZJ 38, GCPII-IN-1, and JB-352.

In certain aspects, the GCPII inhibitor is selected from quisqualate and β-citryl-L-glutamate.

In certain aspects, the GCPII inhibitor is selected from (S)-2-((((S)-5-(4-bromo-2-fluorobenzamido)-1-carboxypentyl)carbamoyl)oxy)pentanedioic acid; and (S)-2-((S)-1-carboxy-3-methylbutylcarbamoyloxy)pentanedioic acid.

In some aspects, the GCPII inhibitor is selected from one or more of 2-PMPA and prodrugs thereof, L-DOPA, D-DOPA, caffeic acid, and prodrugs thereof, a hydroxamate-based prodrug, and a dendrimer 2-PMPA conjugate.

In particular aspects, the 2-PMPA and prodrugs thereof is a compound of formula (Ia) or formula (Ib):

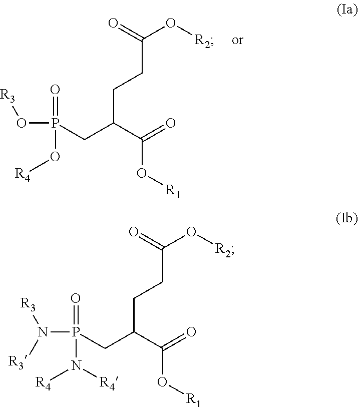

wherein:
each R₁, R₂, R₃, and R₄ is independently selected from the group consisting of H, alkyl, Ar, —(CR₅R₆)ₙ—Ar, —(CR₅R₆)ₙ—O—C(═O)—R₇, —(CR₅R₆)ₙ—C(═O)—O—R₇, —(CR₅R₆)ₙ—O—C(═O)—O—R₇, —(CR₅R₆)ₙ—O—R₇, —(CR₅R₆)ₙ—O—[(CR₅R₆)ₙ—O]ₘ—R₇, —(CR₅R₆)ₙ—Ar—O—C(═O)—R₇, —Ar—C(═O)—O—(CR₅R₆)ₙ—R₇, —(CR₅R₆)ₙ—NR₈R₉, and —(CR₅R₆)ₙ—C(═O)—NR₈R₉;
wherein:
n is an integer from 1 to 20;
m is an integer from 1 to 20;
each R₃′ and R₄′ are independently H or alkyl;
each R₅ and R₆ is independently selected from the group consisting of H, alkyl, and alkylaryl;
each R₇ is independently straight chain or branched alkyl;
Ar is aryl, substituted aryl, heteroaryl or substituted heteroaryl; and
R₈ and R₉ are each independently H or alkyl; and
pharmaceutically acceptable salts thereof.
   In certain aspects, the L-DOPA, D-DOPA, caffeic acid, and prodrugs thereof comprise a compound of formula (II):

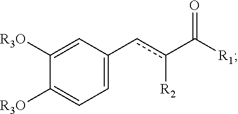

wherein:
[symbol] indicates that the bond can be a single or a double bond;
R₁ is:
—OR₅, wherein R₅ is selected from the group consisting of H, C₁-C₈ alkyl, and —O—(CH₂)ₙ—R₆, wherein n is an integer selected from the group consisting of 0, 1, 2, 3, 4, 5, 6, 7, and 8 and R₆ is substituted or unsubstituted aryl or heteroaryl; or
—NR₇R₈, wherein R₇ and R₈ are each independently selected from the group consisting of H, C₁-C₄ alkyl, C₃-C₆ cycloalkyl, C₁-C₈ alkoxyl, unsubstituted or substituted aryl or heteroaryl, —(CH₂)ₘ—R₉, wherein R₉ is —OR₁₀ or CHX₂, wherein R₁₀ is H or C₁-C₄ alkyl, and each X is halogen, and —(CH₂)ₘ—CH(NH₂)(COOH), wherein each m is independently an integer selected from the group consisting of 0, 1, 2, 3, 4, 5, 6, 7, and 8;
R₂ is H or —NR₁₁R₁₂, wherein R₁₁ and R₁₂ are each independently selected from the group consisting of H, C₁-C₄ alkyl, and —C(═O)—R₁₃, wherein R₁₃ is C₁-C₄ alkyl or —C(NH₂)—(CH₂)ₚ—R₁₄, wherein R₁₄ is C₁-C₄ alkyl or —NR₁₅R₁₆, wherein R₁₅ and R₁₆ are each H or C₁-C₄ alkyl, and p is an integer selected from the group consisting of 0, 1, 2, 3, 4, 5, 6, 7, and 8;
R₃ and R₄ are each independently H or —C(═O)—R₁₇, wherein R₁₇ is C₁-C₈ alkyl or —(CH₂)ₜ—O—C(═O)—O—R₁₈, wherein R₁₈ is C₁-C₈ alkyl, and t is an integer selected from the group consisting of 0, 1, 2, 3, 4, 5, 6, 7, and 8; and
stereoisomers and pharmaceutically acceptable salts thereof.

In certain aspects, the hydroxamate-based prodrug comprises a compound of formula (III):

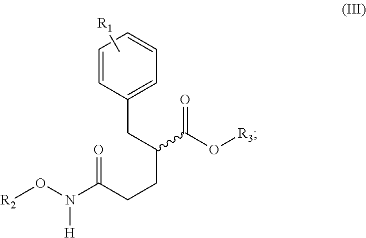

wherein:
R₁ is selected from the group consisting of —C(═O)—O—R₄ and —Ar—C(═O)—O—R₄;
R₂ is selected from the group consisting of substituted and unsubstituted C₁-C₆ alkyl, substituted and unsubstituted C₃-C₈ cycloalkyl, substituted and unsubstituted C₆-C₁₂ aryl, substituted and unsubstituted C₆-C₁₂ heteroaryl, —(CR₅R₆)ₙ—R₇, —C(═O)—O—R₇, —C(═O)—R₇, —C(═O)—NR₇R₈, —(CR₅R₆)ₙ—O—C(═O)—O—R₇, —(CR₅R₆)ₙ—Ar—O—C(═O)—R₇; R₃ is selected from the group consisting of H, substituted and unsubstituted C₁-C₆ alkyl, substituted and unsubstituted C₃-C₁₂ cycloalkyl, substituted and unsubstituted C₆-C₁₂ aryl, substituted and unsubstituted C₅-C₁₂ heteroaryl; R₄ is selected from the group consisting of H, substituted and unsubstituted C₁-C₆ alkyl, substituted and unsubstituted C₃-C₁₂ cycloalkyl, substituted and unsubstituted C₆-C₁₂ aryl, substituted and unsubstituted C₅-C₁₂ heteroaryl, —(CR₅R₆)ₙ—O—C(═O)—O—R₉, and —(CR₅R₆)ₙ—Ar—O—C(═O)—R₉; each R₅ and R₆ is independently selected from the group consisting of H, C₁-C₁₀ alkyl, and C₆-C₁₂ aralkyl; R₇ is selected from the group consisting of H, and substituted and unsubstituted C₁-C₁₀ alkyl, substituted and unsubstituted C₁-C₁₀ heteroalkyl, substituted and unsubstituted C₃-C₁₆ cycloalkyl, substituted and unsubstituted C₃-C₁₂ cycloheteroalkyl, substituted and unsubstituted C₃-C₁₂ cycloheteroalkenyl, substituted and unsubstituted C₆-C₁₂ aryl, substituted and unsubstituted C₆-C₁₂ heteroaryl, and substituted and unsubstituted C₆-C₁₂ aralkyl; R₈ is selected from the group consisting of H, and substituted and unsubstituted C₁-C₆ alkyl; R₉ is selected from the group consisting of H, and substituted and unsubstituted C₁-C₆ alkyl; n is an integer selected from the group consisting of 0, 1, 2, 3, 4, 5, and 6; Ar is selected from the group consisting of substituted and unsubstituted C₆-C₁₂ aryl, and substituted and unsubstituted C₆-C₁₂ heteroaryl; and stereoisomers and pharmaceutically acceptable salts thereof.

In certain aspects, the dendrimer 2-PMPA conjugate comprises one or more dendrimers selected from the group consisting of polyamidoamine (PAMAM), polypropyiamine (POPAM), poly(propylene imine) (PPI), polyethylenimine, polylysine, polyester, iptycene, aliphatic poly(ether), aromatic polyether dendrimers, and combinations thereof. In particular aspects, the dendrimer comprises a generation-4 (G4) to generation-10 (G10) PAMAM dendrimer having terminal group selected from the group consisting of a carboxylic group, an amine group, and a hydroxyl group. In yet more particular aspects, the dendrimer 2-PMPA conjugate comprises a generation-4 through generation-10 hydroxyl-terminated PAMAM dendrimer covalently linked to 2-PMPA through, in some aspects, a disulfide bridge.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

The patent or application file contains at least one drawing executed in color. Copies of this patent or patent application publication with color drawing(s) will be provided by the Office upon request and payment of the necessary fee.

Having thus described the presently disclosed subject matter in general terms, reference will now be made to the accompanying Figures, which are not necessarily drawn to scale, and wherein:

FIG. 1 shows a homodimer of human GCPII (crystal structure) tethered to the biological membrane. Left panel—residing at the plasma membrane of astrocytes/Schwann cells, GCPII catabolizes NAAG, the most prevalent peptidic neurotransmitter in the mammalian nervous system. N-acetylaspartate and glutamate, the reaction products, are selectively transported into glial cells, metabolized and reused for NAAG synthesis in neurons (prior art; modified from Bařrinka et al., 2012;

FIG. 2 demonstrates that excess glutamate causes axonal pruning in the developing peripheral nervous system (prior art; from Personius et al., 2016;

FIG. 3A, FIG. 3B, FIG. 3C, FIG. 3D, FIG. 3E, and FIG. 3F demonstrate that inhibiting elevated GCPII in an ALS mouse model delayed motor function loss and denervation, Tallon et al. 2022;

FIG. 4 demonstrates that aged and ALS mouse muscles are similarly denervated (prior art; Valdez et al., 2012);

FIG. 5A, FIG. 5B, and FIG. 5C demonstrate that GCPII protein and activity levels are increased in muscle from aged mice. FIG. 5A and FIG. 5B demonstrate that GCPII protein expression is increased in 20-month-old WT mice. FIG. 5C demonstrates that GCPII enzymatic activity is increased in 20-month-old WT mice (N=3-6/group);

FIG. 6A, FIG. 6B, FIG. 6C, FIG. 6D, FIG. 6E, and FIG. 6F show that GCPII is expressed on activated macrophages in aged muscle;

FIG. 7A, FIG. 7B, and FIG. 7C show that 2-PMPA treatment delays motor function loss and frailty in aged animals. FIG. 7A shows that CMAP decline is reduced after 20-weeks treatment. FIG. 7B shows that hind limb grip strength decline is reduced after 20-weeks treatment. FIG. 7C shows that ambulatory movement is increased after 20-weeks treatment. FIG. 7D shows that frailty index scores are reduced after 20-weeks treatment (N=48-50/group);

FIG. 8A, FIG. 8B, FIG. 8C, FIG. 8D, and FIG. 8E demonstrates that muscle wasting is delayed in 2-PMPA treated aged animals. FIG. 8A, FIG. 8B, FIG. 8C, and FIG. 8D demonstrate that 8-weeks treatment of 2-PMPA improved whole calf muscle wasting as measured by MRI. FIG. 8E demonstrates that 20-weeks of 2-PMPA treatment improved gastrocnemius muscle wasting as measured by dissected muscle weight (N=8-12/group);

FIG. 9A and FIG. 9B demonstrate that 2-PMPA treatment prolongs survival and delays weight loss. FIG. 9A demonstrates that 2-PMPA mice lost less body weight after 20 weeks on drug. FIG. 9B demonstrates that 2-PMPA treated mice survived longer than vehicle mice (N=48-50/group);

FIG. 10A, FIG. 10B, FIG. 10C, and FIG. 10D demonstrate that 20 weeks of 2-PMPA treatment reduces several circulating pro-inflammatory cytokines (N=8/group); and

FIG. 11A and FIG. 11B demonstrate that 2-PMPA treatment significantly increased gastrocnemius muscle fiber cross sectional area. FIG. 11A is a histogram of the distribution of fiber cross sectional areas in young mice treated with vehicle, old mice treated with vehicle, and old mice treated with 2-PMPA. Both old groups had a leftward shift towards a greater proportion of smaller fibers compared to young mice. 2-PMPA treatment caused a rightward shift towards slightly larger fiber size compared to old mice treated with vehicle. FIG. 11B is the mean cross sectional area (CSA) of gastrocnemius muscle fibers. Both old mice treated with vehicle and old mice treated with 2-PMPA had significantly smaller muscle fibers than young mice treated with vehicle. 2-PMPA treated old mice had significantly larger fiber CSA than vehicle treated old mice. Bars represent±SEM. ** p<0.01, **** p<0.0001.

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

### I. GCPII Inhibition for the Treatment of Sarcopenia and Aging

In some embodiments, the presently disclosed subject matter demonstrates that inhibition of the enzyme glutamate carboxypeptidase II (GCPII) is a potential therapeutic for the treatment of age-related sarcopenia and aging. Sarcopenia is a common condition associated with aging where there is a substantial loss of muscle mass and function, resulting in lowered quality of life, increased fall risk, and associated with poorer health outcomes.

Glutamate carboxypeptidase II (GCPII) is a 94 kD class II membrane bound zinc metalloenzyme which catalyzes the hydrolysis of the abundant neuropeptide N-acetylaspartylglutamate (NAAG) to glutamate. GCPII is a well-established therapeutic target in neurological diseases wherein excess glutamate is presumed pathogenic.

As used herein, the term “excess,” as in “an excess of GCPII,” refers to a level of GCPII in a subject having or suspected of having a disease, disorder, or condition associated with GCPII compared to a level of GCPII in a normal subject, i.e., a subject who does not have or is not suspected of having a disease, disorder, or condition associated with excess GCPII, such as an increase of approximately 50%, 100%, 200%, 300%, 400%, 500%, or more.

In some embodiments, performing the presently disclosed method results in inhibiting excess GCPII activity in a subject. In other embodiments, performing the presently disclosed method results in inhibition of GCPII enzyme activity. As used herein, the term “inhibit,” and grammatical derivations thereof, means to decrease or diminish the excess GCPII activity found in a subject. The term “inhibit” also may mean to decrease, suppress, attenuate, diminish, arrest, or stabilize the development or progression of a disease, disorder, or condition. Inhibition may occur, for e.g., by at least 10%, 20%, 30%, 40%, 50%, 60%, 70%, 80%, 90%, 95%, 98%, 99%, or even 100% compared to an untreated control subject or a subject without the disease or disorder.

As used herein, an “inhibitor” of GCPII is a molecule that generally inhibits or decreases the activity of GCPII. In some embodiments, small molecule GCPII inhibitors, directly or indirectly, increase extracellular NAAG and decrease extracellular glutamate. The inhibitor may interact with GCPII directly or may interact with another molecule that results in a decrease in the activity of GCPII.

More particularly, the presently disclosed subject matter provides a method for treating age-related sarcopenia and/or enhancing longevity, the method comprising administering to a subject in need of treatment thereof one or more GCPII inhibitors. In more particular embodiments, the GCPII inhibitor is selected from one or more of 2-PMPA and prodrugs thereof, L-DOPA, D-DOPA, caffeic acid, and prodrugs thereof, a hydroxamate-based prodrug, and a dendrimer 2-PMPA conjugate.

### A. Representative GCPII Inhibitors

GCPII inhibitors generally fall into the following representative classes including, but not limited to, phosphonates, phosphinates, phosphoramidates, thiols, hydroxamates, and urea-glutamates:

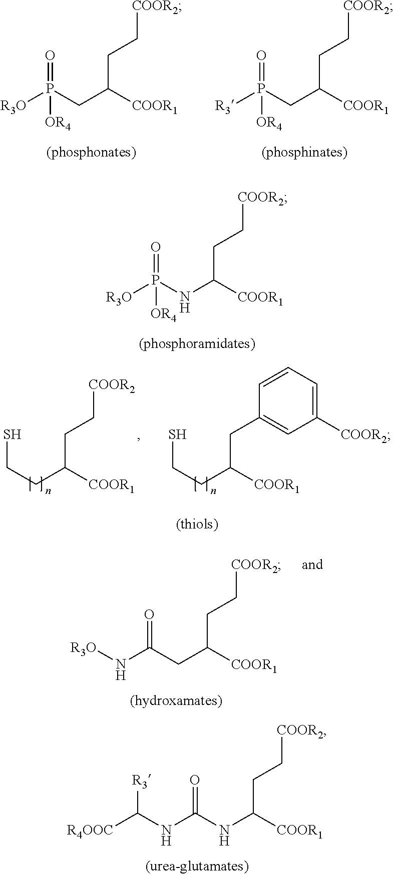

wherein R₁, R₂, R₃, R′₃, and R₄ are substituent groups as defined herein. See, for example, Vornov e al., 20; Pastorino et al. 2020; Gourni and Henrisken, 2017; Bařinka et al., 2012.

Known GCPII inhibitors representative of these general classes include 2-(phosphonomethyl) pentanedioic acid (2-PMPA) (phosphonates), 2-(3-mercaptopropyl)pentanedioic acid (2-MPPA) (thiols), 2-(2-(hydroxyamino)-2-oxoethyl)pentanedioic acid (JHU 241) (hydroxamates), see also Rais et al., 2017, for other hydroxamate-based glutamate GCPII inhibitors, including, 4-carboxy-alpha-[3-(hydroxyamino)-3-oxopropyl]-benzenepropanoic acid, and N—[N—[(S)]-1,3-dicarboxypropyl]carbamoyl]-L-leucine (ZJ-43) (urea-glutamates):

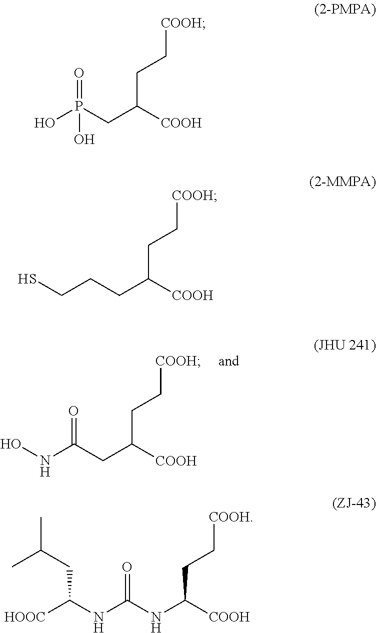 [SMILES: O=C(O)CCC(CP(=O)(O)O)C(=O)O ; O=C(O)CCC(CCCS)C(=O)O ; O=C(O)CCC(CC(=O)NO)C(=O)O ; CC(C)C[C@H](NC(=O)N[C@@H](CCC(=O)O)C(=O)O)C(=O)O]

See, for example, Vornov et al., 2020.

Other phosphonate-based GCPII inhibitors include GPI-5232, Jackson and Slusher, 2001, and VA-033, Ding et al., 2004:

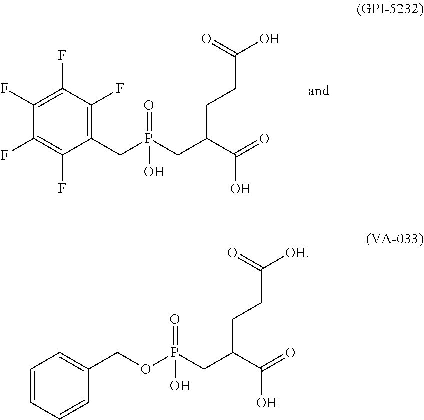 [SMILES: O=C(O)CCC(CP(=O)(O)Cc1c(F)c(F)c(F)c(F)c1F)C(=O)O ; O=C(O)CCC(CP(=O)(O)OCc1ccccc1)C(=O)O]

Representative phosphinate-based GCPII inhibitors include, but are not limited to:
2-[[methylhydroxyphosphinyl]methyl]pentanedioic acid;
2-[[ethylhydroxyphosphinyl]methyl]pentanedioic acid;
2-[[propylhydroxyphosphinyl]methyl]pentanedioic acid;
2-[[butylhydroxyphosphinyl]methyl]pentanedioic acid;
2-[[cyclohexylhydroxyphosphinyl]methyl]pentanedioic acid;
2-[[phenylhydroxyphosphinyl]methyl]pentanedioic acid;
2-[[(phenylmethyl)hydroxyphosphinyl]methyl]pentanedioic acid;
2-[[((2-phenylethyl)methyl)hydroxyphosphinyl]methyl]pentanedioic acid;
2-[[((3-phenylpropyl)methyl)hydroxyphosphinyl]methyl]pentanedioic acid;
2-[[((3-phenylbutyl)methyl)hydroxyphosphinyl]methyl]pentanedioic acid;
2-[[((2-phenylbutyl)methyl)hydroxyphosphinyl]methyl]pentanedioic acid;
2-[[(4-phenylbutyl)hydroxyphosphinyl]methyl]pentanedioic acid;
2-[[(aminomethyl)hydroxyphosphinyl]methyl]pentanedioic acid;
7-(L-2-amino-2-carboxyethylthio)-2-(2,2-dimethylcyclopropanecarboxamide)-2-heptenoic acid; 2-(phosphonomethyl)pentanedioic acid;
N-[methylhydroxyphosphinyl]glutamic acid;
N-[ethylhydroxyphosphinyl]glutamic acid;
N-[propylhydroxyphosphinyl]glutamic acid;
N-[butylhydroxyphosphinyl]glutamic acid;
N-[phenylhydroxyphosphinyl]glutamic acid; and
N-[(phenylmethyl)hydroxyphosphinyl]glutamic acid.

See U.S. Pat. No. 11,167,049, for Organ protection in PSMA-targeted radionuclide therapy of prostate cancer, to Babich et al., issued Nov. 9, 2021, which is incorporated herein by reference in its entirety.

Representative thiol-based GCPII inhibitors include 3-(2-mercaptoethyl)biphenyl-2,3-dicarboxylic acid (E2072) and GPI-5693:

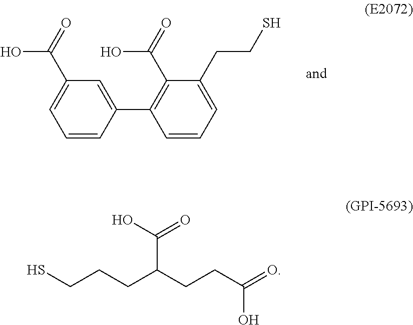 [SMILES: O=C(O)c1cccc(-c2cccc(CCS)c2C(=O)O)c1 ; O=C(O)CCC(CCCS)C(=O)O]

See Wozniak et al., 2012; Slusher et al., 2001. Other thiol-based are provided in Bařinka et al., 2012.

Other representative GCPII inhibitors include quisqualate and β-citryl-L-glutamate:

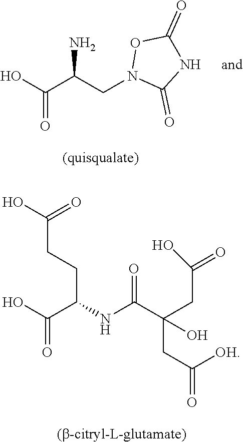 [SMILES: N[C@@H](Cn1oc(=O)[nH]c1=O)C(=O)O ; O=C(O)CC[C@H](NC(=O)C(O)(CC(=O)O)CC(=O)O)C(=O)O]

### See Knedlik et al., 2017.

In some embodiments, the GCPII inhibitor includes:
(S)-2-((N—((S)-1,2-dicarboxyethyl)sulfamoyl)amino)pentanedioic acid:

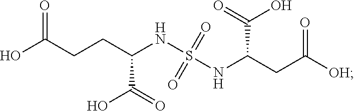 [SMILES: O=C(O)CC[C@H](NS(=O)(=O)N[C@@H](CC(=O)O)C(=O)O)C(=O)O]

(S)-2-((((S)-5-(4-bromo-2-fluorobenzamido)-1-carboxypentyl)carbamoyl)oxy)pentanedioic acid:

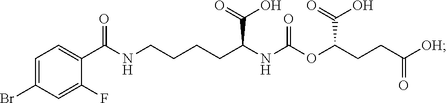 [SMILES: O=C(O)CC[C@H](OC(=O)N[C@@H](CCCCNC(=O)c1ccc(Br)cc1F)C(=O)O)C(=O)O]

(S)-2-((S)-1-carboxy-3-methylbutylcarbamoyloxy)pentanedioic acid:

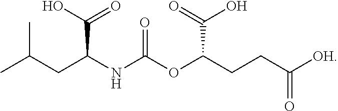 [SMILES: CC(C)C[C@H](NC(=O)O[C@@H](CCC(=O)O)C(=O)O)C(=O)O]

Urea-glutamate based GCPII inhibitors include MIP-1555, MIP-1519, MIP-1545, MIP-1427, MIP-1428, MIP-1379, MIP-1072, MIP-1095, MIP-1558, MIP-1405, and MIP-1404. See U.S. Pat. No. 11,167,049, for Organ protection in PSMA-targeted radionuclide therapy of prostate cancer, to Babich et al., issued Nov. 9, 2021, which is incorporated herein by reference in its entirety.

Other urea-glutamate based GCPII inhibitors include PSMA I&T, Weineisen et al., 2015, PSMA-617, Benešová et al., 2015, PSMA-11, Eder et al., 2012, DCIBzL, Chen et al., 2008, ¹⁸F-DCFPyl, Chen et al., 2011, ZJ 38, GCPII-IN-1, and JB-352, Knedlik et al. 2017:

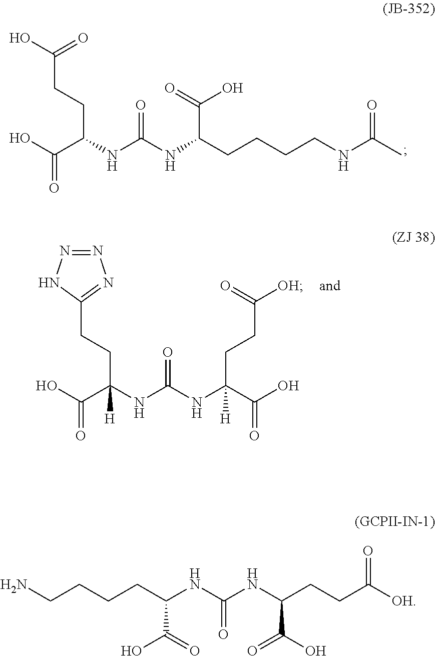 [SMILES: CC(=O)NCCCC[C@H](NC(=O)N[C@@H](CCC(=O)O)C(=O)O)C(=O)O ; O=C(O)CC[C@H](NC(=O)N[C@@H](CCc1nnn[nH]1)C(=O)O)C(=O)O ; NCCCC[C@H](NC(=O)N[C@@H](CCC(=O)O)C(=O)O)C(=O)O]

### B. 2-PMPA and Prodrugs Thereof

In some embodiments, the GCPII inhibitor is 2-PMPA or a prodrug thereof. Representative prodrugs of 2-PMPA are disclosed in International PCT Patent Application Publication No. WO2016022827 for Prodrugs of Prostate Specific Membrane Antigen (PSMA) Inhibitor, to Slusher et al., published Feb. 11, 2016, which is incorporated by reference in its entirety, in particular page 9, line 19, through page 25, line 12.

TABLE 1
Structures of Representative 2-PMPA Prodrugs and Metabolic
Products

| IOCB No./ | | |
|---|---|---|
| Compound No. | Structure | MW |
| 2-PMPA | 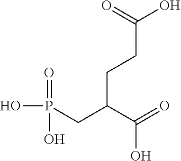 [SMILES: O=C(O)CCC(CP(=O)(O)O)C(=O)O] | 226.12 |
| TT-140113 JHU 2106 | 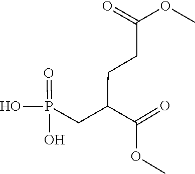 [SMILES: COC(=O)CCC(CP(=O)(O)O)C(=O)OC] | 254.17 |
| MK-797 JHU 2236 | 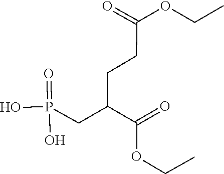 [SMILES: CCOC(=O)CCC(CP(=O)(O)O)C(=O)OCC] | 282.23 |
| MK-801 JHU 2263 | 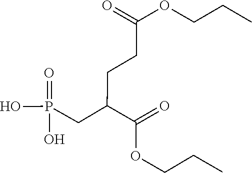 [SMILES: CCCOC(=O)CCC(CP(=O)(O)O)C(=O)OCCC] | 310.28 |
| | 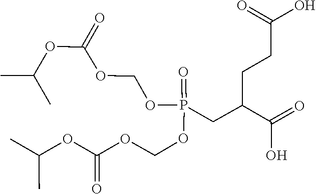 [SMILES: CC(C)OC(=O)OCOP(=O)(CC(CCC(=O)O)C(=O)O)OCOC(=O)OC(C)C] | 458.35 |
| | 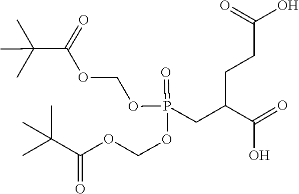 [SMILES: CC(C)(C)C(=O)OCOP(=O)(CC(CCC(=O)O)C(=O)O)OCOC(=O)C(C)(C)C] | 454.41 |
| TT-010213 JHU 2110 | 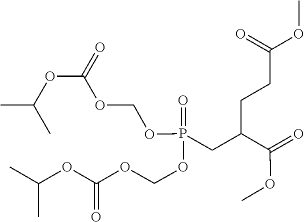 [SMILES: COC(=O)CCC(CP(=O)(OCOC(=O)OC(C)C)OCOC(=O)OC(C)C)C(=O)OC] | 486.41 |
| MK-793 JHU 2234 | 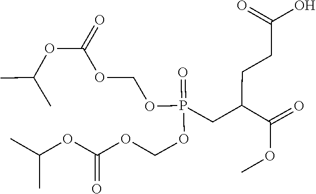 [SMILES: COC(=O)C(CCC(=O)O)CP(=O)(OCOC(=O)OC(C)C)OCOC(=O)OC(C)C] | 472.38 |
| TT-150313 JHU 2201 | 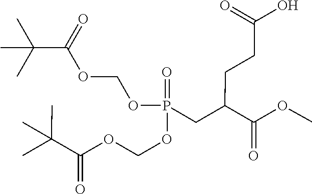 [SMILES: COC(=O)C(CCC(=O)O)CP(=O)(OCOC(=O)C(C)(C)C)OCOC(=O)C(C)(C)C] | 468.43 |
| | 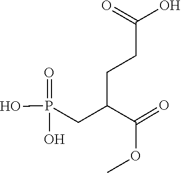 | 240.15 |
| JAM0186 2-PMPATRIS- POC | 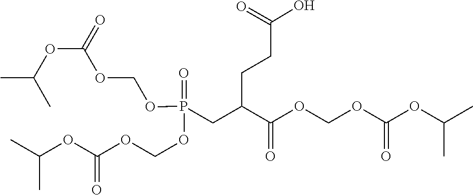 [SMILES: CC(C)OC(=O)OCOC(=O)C(CCC(=O)O)CP(=O)(OCOC(=O)OC(C)C)OCOC(=O)OC(C)C] | 574.47 |
| | 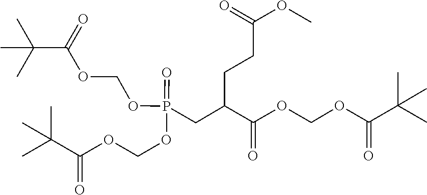 [SMILES: COC(=O)CCC(CP(=O)(OCOC(=O)C(C)(C)C)OCOC(=O)C(C)(C)C)C(=O)OCOC(=O)C(C)(C)C] | 582.57 |
| TT-041212 JHU 2107 | 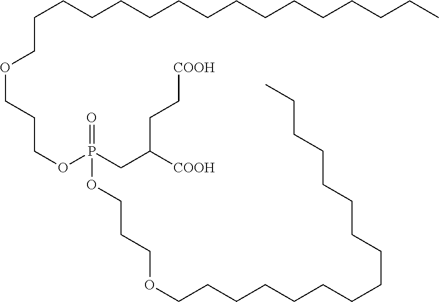 [SMILES: CCCCCCCCCCCCCCCCOCCCOP(=O)(CC(CCC(=O)O)C(=O)O)OCCCOCCCCCCCCCCCCCCCC] | 791.13 |
| TT-250113 JHU 2108 | 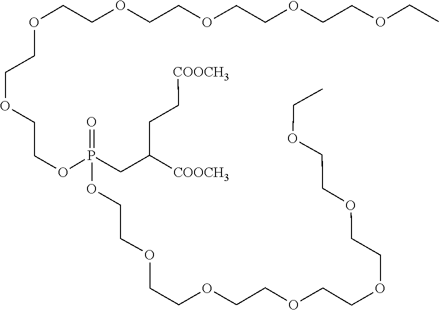 [SMILES: CCOCCOCCOCCOCCOCCOCCOP(=O)(CC(CCC(=O)OC)C(=O)OC)OCCOCCOCCOCCOCCOCCOCC] | 838.90 |
| TT-201212A JHU 2109 | 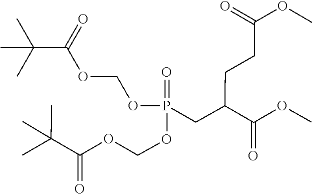 [SMILES: COC(=O)CCC(CP(=O)(OCOC(=O)C(C)(C)C)OCOC(=O)C(C)(C)C)C(=O)OC] | 482.46 |
| TT-010213 JHU 2110 | 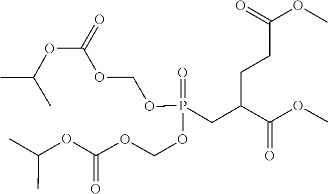 [SMILES: COC(=O)CCC(CP(=O)(OCOC(=O)OC(C)C)OCOC(=O)OC(C)C)C(=O)OC] | 486.40 |
| TT-100113 JHU 2111 | 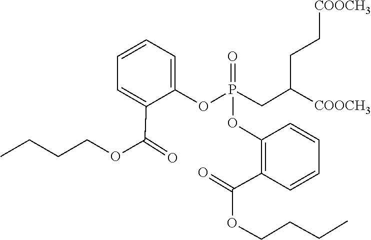 [SMILES: CCCCOC(=O)c1ccccc1OP(=O)(CC(CCC(=O)OC)C(=O)OC)Oc1ccccc1C(=O)OCCCC] | 606.58 |
| TT-280113 JHU 2112 | 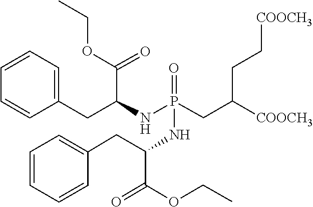 [SMILES: CCOC(=O)[C@H](Cc1ccccc1)NP(=O)(CC(CCC(=O)OC)C(=O)OC)N[C@@H](Cc1ccccc1)C(=O)OCC] | 604.60 |
| MK798 JHU 2237 | 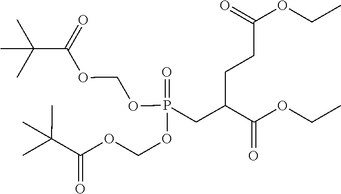 [SMILES: CCOC(=O)CCC(CP(=O)(OCOC(=O)C(C)(C)C)OCOC(=O)C(C)(C)C)C(=O)OCC] | 510.51 |
| MK804 | | 482.46 |
| JHU 2264 | | |
| MK806 | | 486.40 |
| JHU 2265 | | |
| MK-795 JHU 2235 | 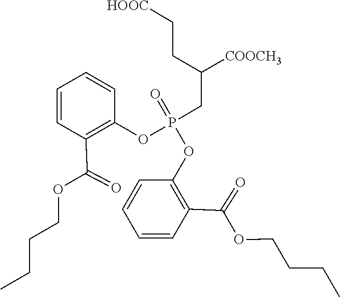 [SMILES: CCCCOC(=O)c1ccccc1OP(=O)(CC(CCC(=O)O)C(=O)OC)Oc1ccccc1C(=O)OCCCC] | 592.57 |
| MK-799 JHU 2238 | 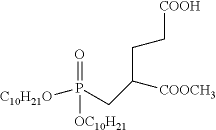 | 520.68 |
| JAM0168 | | 568.55 |
| 2-PMPA TRIS | | |
| POM | | |
| JAM0195 JHU 2609 | 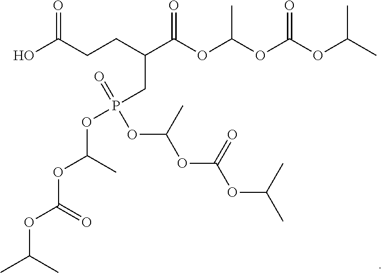 [SMILES: CC(C)OC(=O)OC(C)OC(=O)C(CCC(=O)O)CP(=O)(OC(C)OC(=O)OC(C)C)OC(C)OC(=O)OC(C)C] | 616.55 |
| JAM0191 JHU 2608 | 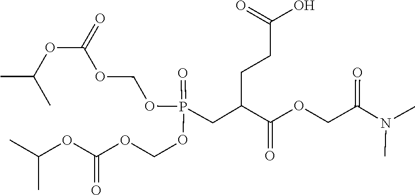 [SMILES: CC(C)OC(=O)OCOP(=O)(CC(CCC(=O)O)C(=O)OCC(=O)N(C)C)OCOC(=O)OC(C)C] | 543.46 |
| JAM0196 JHU 2610 | 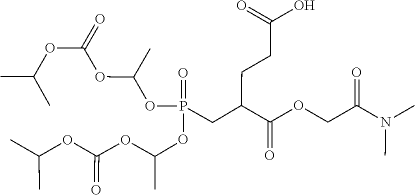 [SMILES: CC(C)OC(=O)OC(C)OP(=O)(CC(CCC(=O)O)C(=O)OCC(=O)N(C)C)OC(C)OC(=O)OC(C)C] | 571.51 |
| LTP023 | 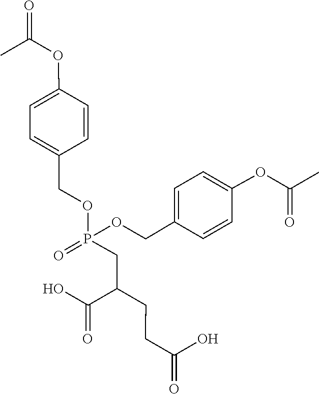 [SMILES: CC(=O)Oc1ccc(COP(=O)(CC(CCC(=O)O)C(=O)O)OCc2ccc(OC(C)=O)cc2)cc1] | 522.44 |
| LTP120 | 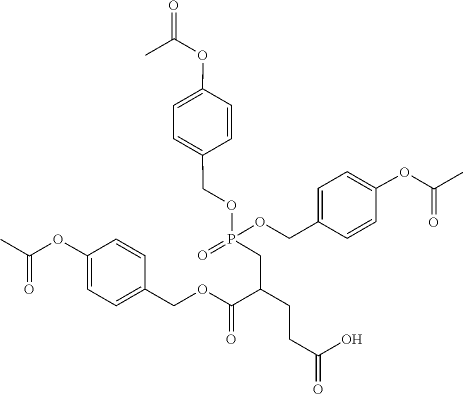 [SMILES: CC(=O)Oc1ccc(COC(=O)C(CCC(=O)O)CP(=O)(OCc2ccc(OC(C)=O)cc2)OCc2ccc(OC(C)=O)cc2)cc1] | 670.60 |
| LTP124 | 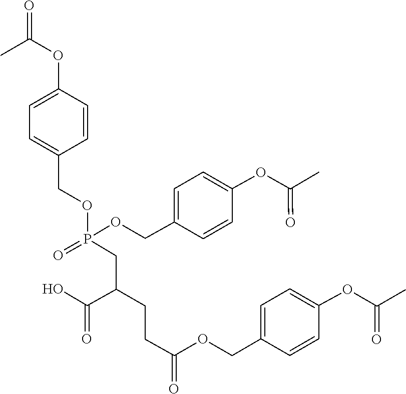 [SMILES: CC(=O)Oc1ccc(COC(=O)CCC(CP(=O)(OCc2ccc(OC(C)=O)cc2)OCc2ccc(OC(C)=O)cc2)C(=O)O)cc1] | 670.60 |
| JAM0388H | 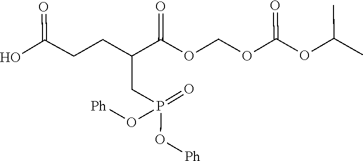 [SMILES: CC(C)OC(=O)OCOC(=O)C(CCC(=O)O)CP(=O)(Oc1ccccc1)Oc1ccccc1] | 494.43 |
| JAM0341H | 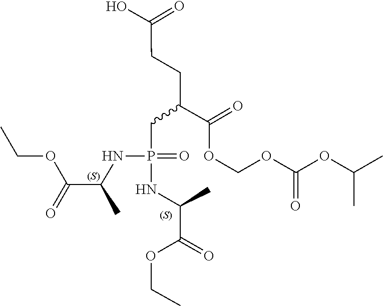 | 540.50 |
| TT-120814 | 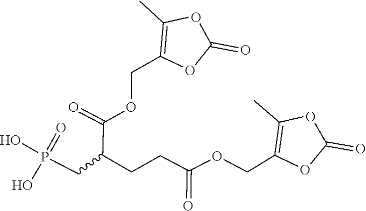 [SMILES: Cc1oc(=O)oc1COC(=O)CCC(CP(=O)(O)O)C(=O)OCc1oc(=O)oc1C] | 450.29 |
| TT-200714 | 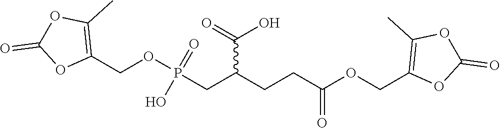 [SMILES: Cc1oc(=O)oc1COC(=O)CCC(CP(=O)(O)OCc1oc(=O)oc1C)C(=O)O] | 450.29 |
| TT-270514 | 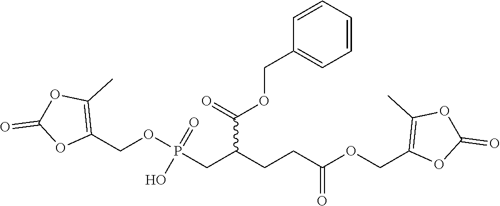 [SMILES: Cc1oc(=O)oc1COC(=O)CCC(CP(=O)(O)OCc1oc(=O)oc1C)C(=O)OCc1ccccc1] | 540.41 |
| TT-011214 | 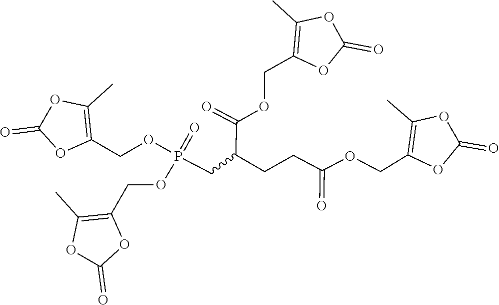 [SMILES: Cc1oc(=O)oc1COC(=O)CCC(CP(=O)(OCc1oc(=O)oc1C)OCc1oc(=O)oc1C)C(=O)OCc1oc(=O)oc1C] | 674.45 |
| TT-110814A | 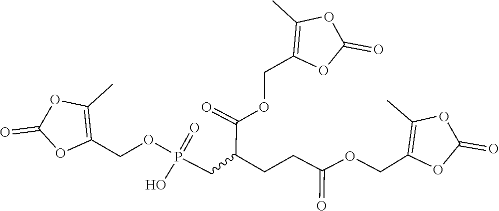 [SMILES: Cc1oc(=O)oc1COC(=O)CCC(CP(=O)(O)OCc1oc(=O)oc1C)C(=O)OCc1oc(=O)oc1C] | 562.37 |

In some embodiments, the presently disclosed subject matter provides a compound of formula (Ia) or formula (Ib):

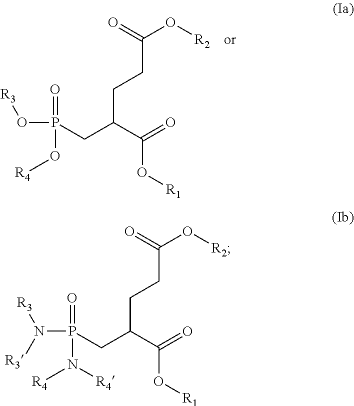

wherein:
each R₁, R₂, R₃, and R₄ is independently selected from the group consisting of H, alkyl, Ar, —(CR₅R₆)ₙ—Ar, —(CR₅R₆)ₙ—O—C(═O)—R₇, —(CR₅R₆)ₙ—C(═O)—O—R₇, —(CR₅R₆)ₙ—O—C(═O)—O—R₇, —(CR₅R₆)ₙ—O—R₇, —(CR₅R₆)ₙ—O—[(CR₅R₆)ₙ—O]ₘ—R₇, —(CR₅R₆)ₙ—Ar—O—C(═O)—R₇, —Ar—C(═O)—O—(CR₅R₆)ₙ—R₇, —(CR₅R₆)ₙ—NR₈R₉, and —(CR₅R₆)ₙ—C(═O)—NR₈R₉;
wherein:
n is an integer from 1 to 20;
m is an integer from 1 to 20;
each R₃′ and R₄′ are independently H or alkyl;
each R₅ and R₆ is independently selected from the group consisting of H, alkyl, and alkylaryl;
each R₇ is independently straight chain or branched alkyl;
Ar is aryl, substituted aryl, heteroaryl or substituted heteroaryl; and
R₈ and R₉ are each independently H or alkyl; and
pharmaceutically acceptable salts thereof.

In particular embodiments, the compound of formula (Ia) is selected from the group consisting of:

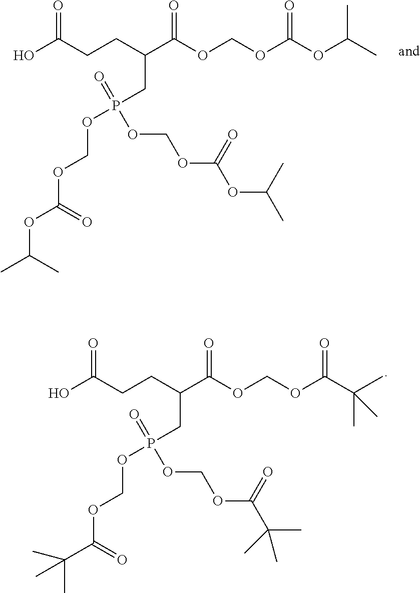

In particular embodiments, the compound of formula (Ia) is selected from the group consisting of:

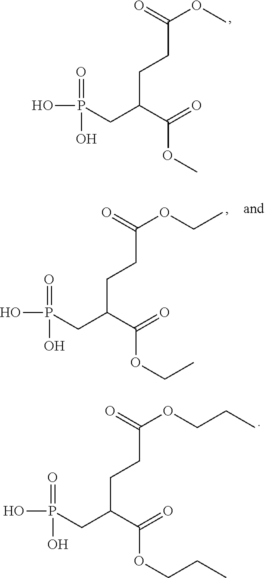

In particular embodiments, the compound of formula (Ia) is selected from the group consisting of:

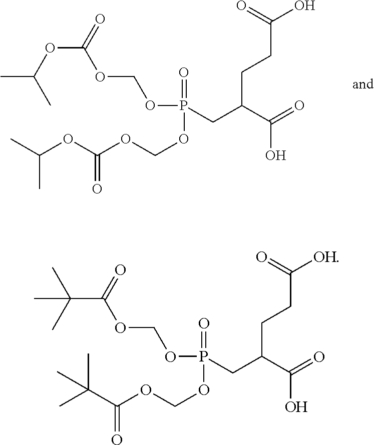 [SMILES: CC(C)OC(=O)OCOP(=O)(CC(CCC(=O)O)C(=O)O)OCOC(=O)OC(C)C ; CC(C)(C)C(=O)OCOP(=O)(CC(CCC(=O)O)C(=O)O)OCOC(=O)C(C)(C)C]

In particular embodiments, the compound of formula (Ia) is selected from the group consisting of:

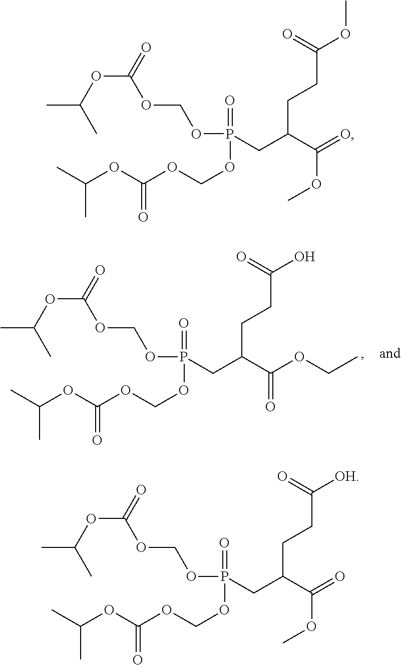

In particular embodiments, the compound of formula (Ia) is:

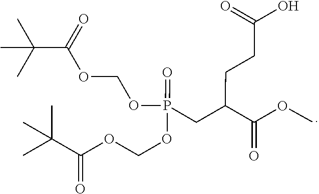 [SMILES: COC(=O)C(CCC(=O)O)CP(=O)(OCOC(=O)C(C)(C)C)OCOC(=O)C(C)(C)C]

In particular embodiments, the compound of formula (Ia) is:

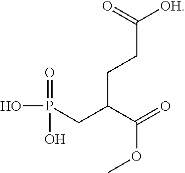

In particular embodiments, the compound of formula (Ia) is:

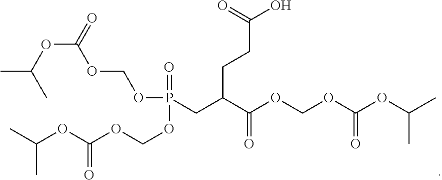 [SMILES: CC(C)OC(=O)OCOC(=O)C(CCC(=O)O)CP(=O)(OCOC(=O)OC(C)C)OCOC(=O)OC(C)C]

In particular embodiments, the compound of formula (Ia) is:

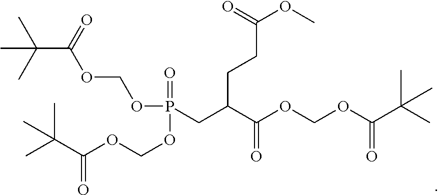 [SMILES: COC(=O)CCC(CP(=O)(OCOC(=O)C(C)(C)C)OCOC(=O)C(C)(C)C)C(=O)OCOC(=O)C(C)(C)C]

In particular embodiments, the compound of formula (Ia) is:

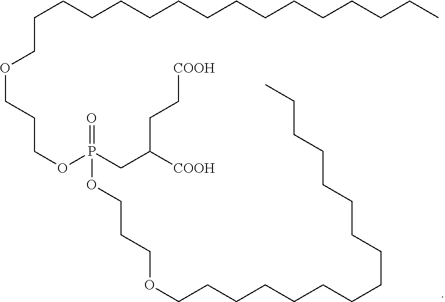 [SMILES: CCCCCCCCCCCCCCCCOCCCOP(=O)(CC(CCC(=O)O)C(=O)O)OCCCOCCCCCCCCCCCCCCCC]

In particular embodiments, the compound of formula (Ia) is:

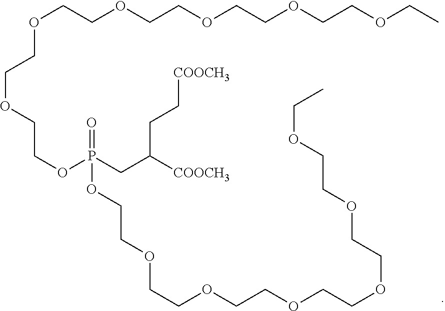 [SMILES: CCOCCOCCOCCOCCOCCOCCOP(=O)(CC(CCC(=O)OC)C(=O)OC)OCCOCCOCCOCCOCCOCCOCC]

In particular embodiments, the compound of formula (Ia) is selected from the group consisting of:

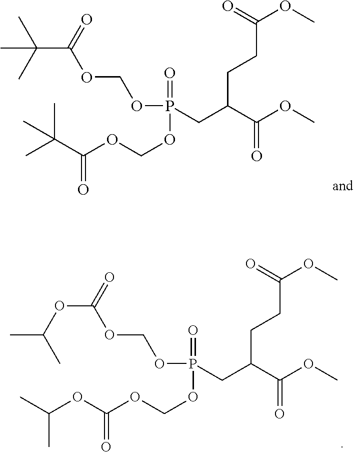

In particular embodiments, the compound of formula (Ia) is:

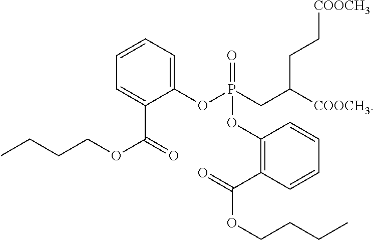 [SMILES: CCCCOC(=O)c1ccccc1OP(=O)(CC(CCC(=O)OC)C(=O)OC)Oc1ccccc1C(=O)OCCCC]

In particular embodiments, the compound of formula (Ia) is:

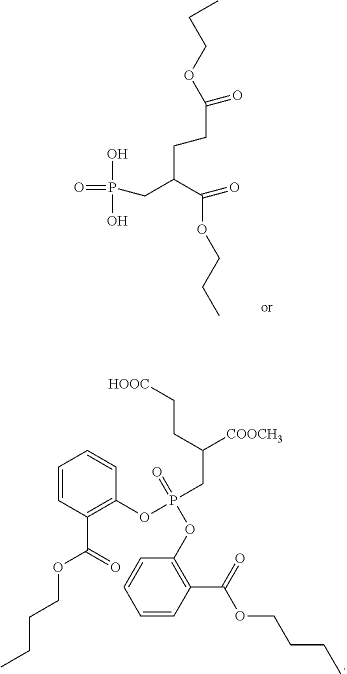 [SMILES: CCCOC(=O)CCC(CP(=O)(O)O)C(=O)OCCC ; CCCCOC(=O)c1ccccc1OP(=O)(CC(CCC(=O)O)C(=O)OC)Oc1ccccc1C(=O)OCCCC]

In particular embodiments, the compound of formula (Ia) is selected from the group consisting of:

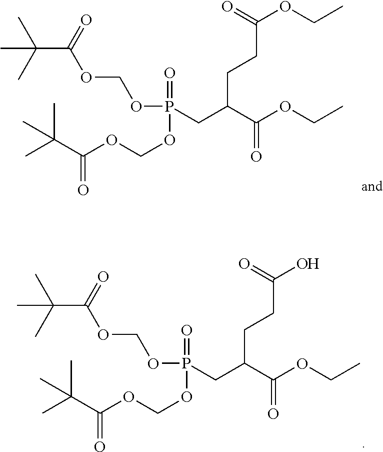

In particular embodiments, the compound of formula (Ia) is:

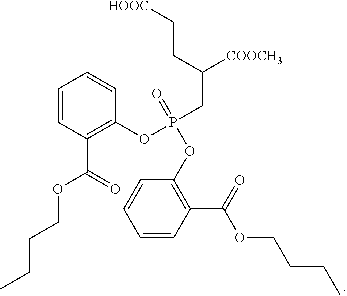 [SMILES: CCCCOC(=O)c1ccccc1OP(=O)(CC(CCC(=O)O)C(=O)OC)Oc1ccccc1C(=O)OCCCC]

In particular embodiments, the compound of formula (Ia) is

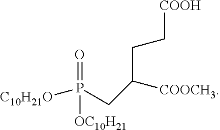

In particular embodiments, the compound of formula (Ia) is

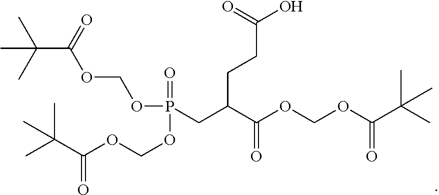 [SMILES: CC(C)(C)C(=O)OCOC(=O)C(CCC(=O)O)CP(=O)(OCOC(=O)C(C)(C)C)OCOC(=O)C(C)(C)C]

In particular embodiments, the compound of formula (Ia) is:

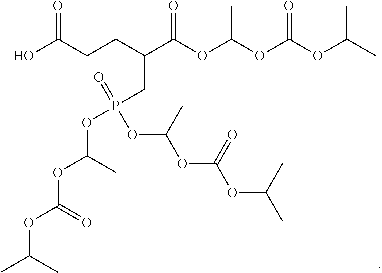 [SMILES: CC(C)OC(=O)OC(C)OC(=O)C(CCC(=O)O)CP(=O)(OC(C)OC(=O)OC(C)C)OC(C)OC(=O)OC(C)C]

In particular embodiments, the compound of formula (Ia) is selected from the group consisting of:

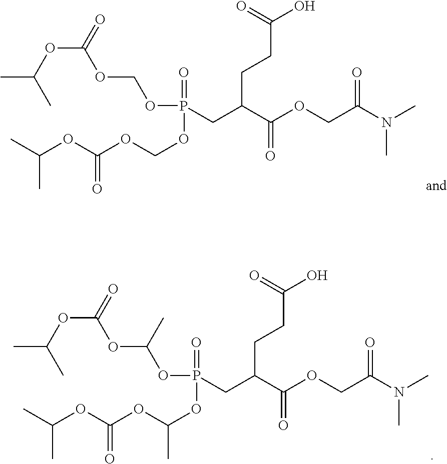

In particular embodiments, the compound of formula (Ia) is selected from the group consisting of:

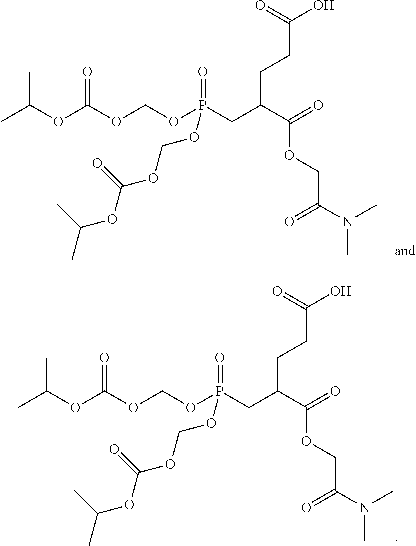

In particular embodiments, the compound of formula (Ia) is selected from the group consisting of:

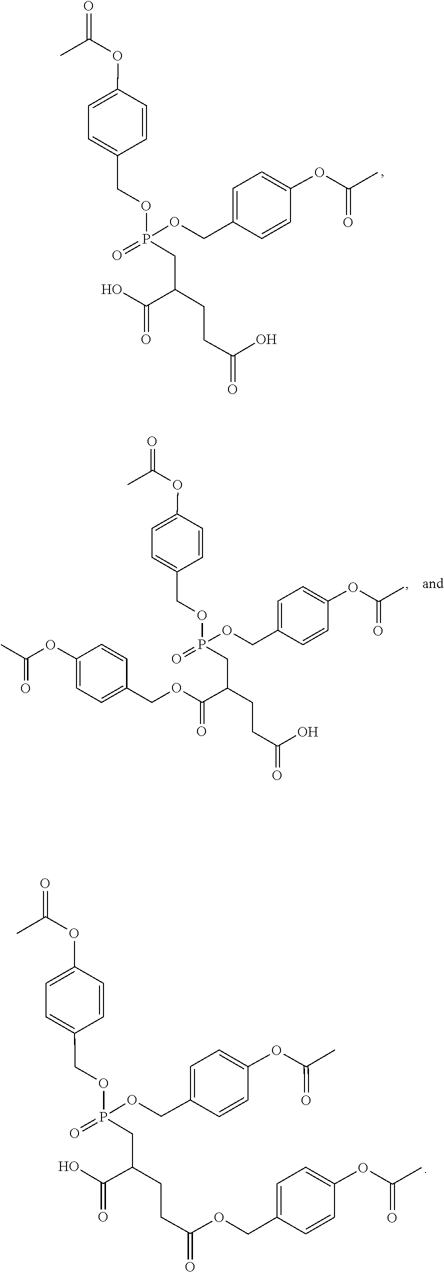

In particular embodiments, the compound of formula (Ia) is:

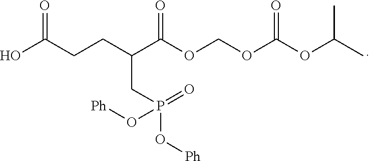 [SMILES: CC(C)OC(=O)OCOC(=O)C(CCC(=O)O)CP(=O)(Oc1ccccc1)Oc1ccccc1]

In particular embodiments, the compound of formula (Ia) is selected from the group consisting of:

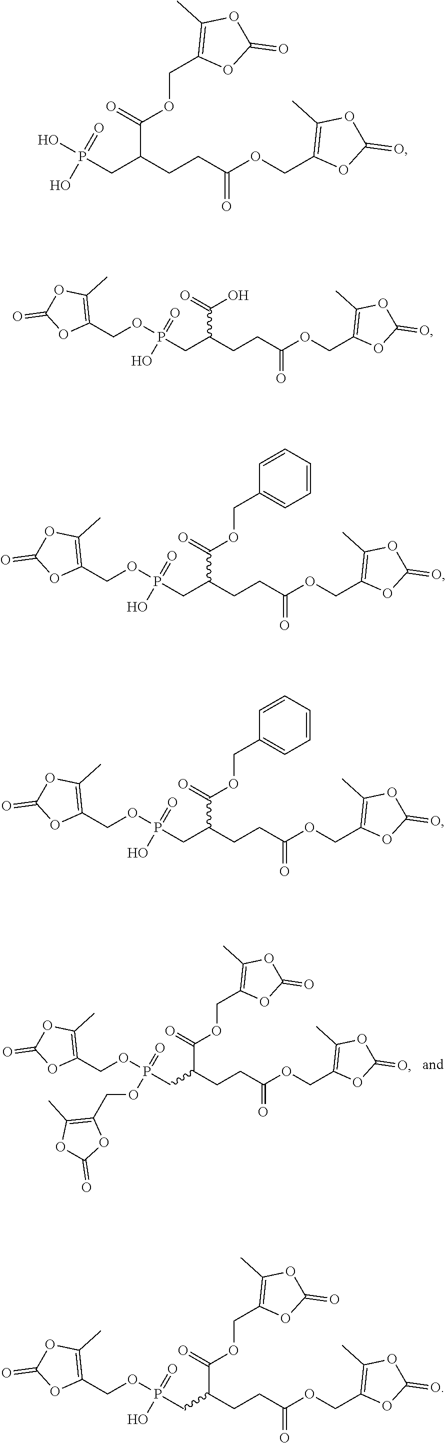 [SMILES: Cc1oc(=O)oc1COC(=O)CCC(CP(=O)(O)O)C(=O)OCc1oc(=O)oc1C ; Cc1oc(=O)oc1COC(=O)CCC(CP(=O)(O)OCc1oc(=O)oc1C)C(=O)O ; Cc1oc(=O)oc1COC(=O)CCC(CP(=O)(O)OCc1oc(=O)oc1C)C(=O)OCc1ccccc1 ; Cc1oc(=O)oc1COC(=O)CCC(CP(=O)(O)OCc1oc(=O)oc1C)C(=O)OCc1ccccc1 ; Cc1oc(=O)oc1COC(=O)CCC(CP(=O)(OCc1oc(=O)oc1C)OCc1oc(=O)oc1C)C(=O)OCc1oc(=O)oc1C ; Cc1oc(=O)oc1COC(=O)CCC(CP(=O)(O)OCc1oc(=O)oc1C)C(=O)OCc1oc(=O)oc1C]

In particular embodiments, the compound of formula (Ib) is:

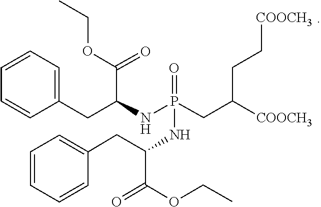 [SMILES: CCOC(=O)[C@H](Cc1ccccc1)NP(=O)(CC(CCC(=O)OC)C(=O)OC)N[C@@H](Cc1ccccc1)C(=O)OCC]

In particular embodiments, the compound of formula (Ib) is:

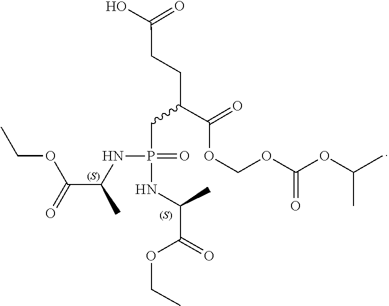

In certain embodiments:
(a) each R₁ is H;
each R₂ is selected from the group consisting of H, alkyl, Ar, —(CR₅R₆)ₙ—Ar, —(CR₅R₆)ₙ—O—C(═O)—R₇, —(CR₅R₆)ₙ—C(═O)—O—R₇, —(CR₅R₆)ₙ—O—C(═O)—O—R₇, —(CR₅R₆)ₙ—O—R₇, —(CR₅R₆)ₙ—O—[(CR₅R₆)ₙ—O]ₘ—R₇, —(CR₅R₆)ₙ—Ar—O—C(═O)—R₇, —Ar—C(═O)—O—(CR₅R₆)ₙ—R₇, —(CR₅R₆)ₙ—NR₈R₉, and —(CR₅R₆)ₙ—C(═O)—NR₈R₉;
each R₃ is selected from the group consisting of H, alkyl, Ar, —(CR₅R₆)ₙ—Ar, —(CR₅R₆)ₙ—O—C(═O)—R₇, —(CR₅R₆)ₙ—C(═O)—O—R₇, —(CR₅R₆)ₙ—O—C(═O)—O—R₇, —(CR₅R₆)ₙ—O—R₇, —(CR₅R₆)ₙ—O—[(CR₅R₆)ₙ—O]ₘ—R₇, —(CR₅R₆)ₙ—Ar—O—C(═O)—R₇, —Ar—C(═O)—O—(CR₅R₆)ₙ—R₇, —(CR₅R₆)ₙ—NR₈R₉, and —(CR₅R₆)ₙ—C(═O)—NR₈R₉; and
each R₄ is selected from the group consisting of —(CR₅R₆)ₙ—O—C(═O)—R₇, —(CR₅R₆)ₙ—C(═O)—O—R₇, —(CR₅R₆)ₙ—O—C(═O)—O—R₇, —(CR₅R₆)ₙ—O—R₇, —(CR₅R₆)ₙ—O—[(CR₅R₆)ₙ—O]ₘ—R₇, —(CR₅R₆)ₙ—Ar—O—C(═O)—R₇, —Ar—C(═O)—O—(CR₅R₆)ₙ—R₇, —(CR₅R₆)ₙ—NR₈R₉, and —(CR₅R₆)ₙ—C(═O)—NR₈R₉;
(b) each R₁ is alkyl;
each R₂ is selected from the group consisting of H, alkyl, Ar, —(CR₅R₆)ₙ—Ar, —(CR₅R₆)ₙ—O—C(═O)—R₇, —(CR₅R₆)ₙ—C(═O)—O—R₇, —(CR₅R₆)ₙ—O—C(═O)—O—R₇, —(CR₅R₆)ₙ—O—R₇, —(CR₅R₆)ₙ—O—[(CR₅R₆)ₙ—O]ₘ—R₇, —(CR₅R₆)ₙ—Ar—O—C(═O)—R₇, —Ar—C(═O)—O—(CR₅R₆)ₙ—R₇, —(CR₅R₆)ₙ—NR₈R₉, and —(CR₅R₆)ₙ—C(═O)—NR₈R₉;
each R₃ is selected from the group consisting of H, alkyl, Ar, —(CR₅R₆)ₙ—Ar, —(CR₅R₆)ₙ—O—C(═O)—R₇, —(CR₅R₆)ₙ—C(═O)—O—R₇, —(CR₅R₆)ₙ—O—C(═O)—O—R₇, —(CR₅R₆)ₙ—O—R₇, —(CR₅R₆)ₙ—O—[(CR₅R₆)ₙ—O]ₘ—R₇, —(CR₅R₆)ₙ—Ar—O—C(═O)—R₇, —Ar—C(═O)—O—(CR₅R₆)ₙ—R₇, —(CR₅R₆)ₙ—NR₈R₉, and —(CR₅R₆)ₙ—C(═O)—NR₈R₉; and
each R₄ is selected from the group consisting of Ar, —(CR₅R₆)ₙ—O—C(═O)—R₇, —(CR₅R₆)ₙ—C(═O)—O—R₇, —(CR₅R₆)ₙ—O—C(═O)—O—R₇, —(CR₅R₆)ₙ—O—R₇, —(CR₅R₆)ₙ—O—[(CR₅R₆)ₙ—O]ₘ—R₇, —(CR₅R₆)ₙ—Ar—O—C(═O)—R₇, —Ar—C(═O)—O—(CR₅R₆)ₙ—R₇, —(CR₅R₆)ₙ—NR₈R₉, and —(CR₅R₆)ₙ—C(═O)—NR₈R₉;
(c) each R₁ is —(CR₅R₆)ₙ—Ar;
each R₂ is selected from the group consisting of H, alkyl, Ar, —(CR₅R₆)ₙ—Ar, —(CR₅R₆)ₙ—O—C(═O)—R₇, —(CR₅R₆)ₙ—C(═O)—O—R₇, —(CR₅R₆)ₙ—O—C(═O)—O—R₇, —(CR₅R₆)ₙ—O—R₇, —(CR₅R₆)ₙ—O—[(CR₅R₆)ₙ—O]ₘ—R₇, —(CR₅R₆)ₙ—Ar—O—C(═O)—R₇, —Ar—C(═O)—O—(CR₅R₆)ₙ—R₇, —(CR₅R₆)ₙ—NR₈R₉, and —(CR₅R₆)ₙ—C(═O)—NR₈R₉;
each R₃ is selected from the group consisting of H, alkyl, Ar, —(CR₅R₆)ₙ—Ar, —(CR₅R₆)ₙ—O—C(═O)—R₇, —(CR₅R₆)ₙ—C(═O)—O—R₇, —(CR₅R₆)ₙ—O—C(═O)—O—R₇, —(CR₅R₆)ₙ—O—R₇, —(CR₅R₆)ₙ—O—[(CR₅R₆)ₙ—O]ₘ—R₇, —(CR₅R₆)ₙ—Ar—O—C(═O)—R₇, —Ar—C(═O)—O—(CR₅R₆)ₙ—R₇, —(CR₅R₆)ₙ—NR₈R₉, and —(CR₅R₆)ₙ—C(═O)—NR₈R₉; and
each R₄ is selected from the group consisting of Ar, —(CR₅R₆)ₙ—O—C(═O)—R₇, —(CR₅R₆)ₙ—C(═O)—O—R₇, —(CR₅R₆)ₙ—O—C(═O)—O—R₇, —(CR₅R₆)ₙ—O—R₇, —(CR₅R₆)ₙ—O—[(CR₅R₆)ₙ—O]ₘ—R₇, —(CR₅R₆)ₙ—Ar—O—C(═O)—R₇, —Ar—C(═O)—O—(CR₅R₆)ₙ—R₇, —(CR₅R₆)ₙ—NR₈R₉, and —(CR₅R₆)ₙ—C(═O)—NR₈R₉; or
(d) each R₁ is selected from Ar, —(CR₅R₆)ₙ—O—C(═O)—R₇, —(CR₅R₆)ₙ—C(═O)—O—R₇, —(CR₅R₆)ₙ—O—C(═O)—O—R₇, —(CR₅R₆)ₙ—O—R₇, —(CR₅R₆)ₙ—O—[(CR₅R₆)ₙ—O]ₘ—R₇, —(CR₅R₆)ₙ—Ar—O—C(═O)—R₇, —Ar—C(═O)—O—(CR₅R₆)ₙ—R₇, —(CR₅R₆)ₙ—NR₈R₉, and —(CR₅R₆)ₙ—C(═O)—NR₈R₉;
each R₂ is selected from the group consisting of H, alkyl, Ar, —(CR₅R₆)ₙ—Ar, —(CR₅R₆)ₙ—O—C(═O)—R₇, —(CR₅R₆)ₙ—C(═O)—O—R₇, —(CR₅R₆)ₙ—O—C(═O)—O—R₇, —(CR₅R₆)ₙ—O—R₇, —(CR₅R₆)ₙ—O—[(CR₅R₆)ₙ—O]ₘ—R₇, —(CR₅R₆)ₙ—Ar—O—C(═O)—R₇, —Ar—C(═O)—O—(CR₅R₆)ₙ—R₇, —(CR₅R₆)ₙ—NR₈R₉, and —(CR₅R₆)ₙ—C(═O)—NR₈R₉;
each R₃ is selected from the group consisting of H, alkyl, Ar, —(CR₅R₆)ₙ—Ar, —(CR₅R₆)ₙ—O—C(═O)—R₇, —(CR₅R₆)ₙ—C(═O)—O—R₇, —(CR₅R₆)ₙ—O—C(═O)—O—R₇, —(CR₅R₆)ₙ—O—R₇, —(CR₅R₆)ₙ—O—[(CR₅R₆)ₙ—O]ₘ—R₇, —(CR₅R₆)ₙ—Ar—O—C(═O)—R₇, —Ar—C(═O)—O—(CR₅R₆)ₙ—R₇, —(CR₅R₆)ₙ—NR₈R₉, and —(CR₅R₆)ₙ—C(═O)—NR₈R₉; and
each R₄ is selected from the group consisting of H, alkyl, Ar, —(CR₅R₆)ₙ—Ar, —(CR₅R₆)ₙ—O—C(═O)—R₇, —(CR₅R₆)ₙ—C(═O)—O—R₇, —(CR₅R₆)ₙ—O—C(═O)—O—R₇, —(CR₅R₆)ₙ—O—R₇, —(CR₅R₆)ₙ—O—[(CR₅R₆)ₙ—O]ₘ—R₇, —(CR₅R₆)ₙ—Ar—O—C(═O)—R₇, —Ar—C(═O)—O—(CR₅R₆)ₙ—R₇, —(CR₅R₆)ₙ—NR₈R₉, and —(CR₅R₆)ₙ—C(═O)—NR₈R₉;
wherein:
each n is an integer from 1 to 20;
each m is an integer from 1 to 20;
each R₅ and R₆ is independently selected from the group consisting of H, alkyl, and alkylaryl;
each R₇ is independently straight chain or branched alkyl;
each Ar is aryl, substituted aryl, heteroaryl or substituted heteroaryl;
each R₈ and R₉ are independently H or alkyl; and
each R₃′ and R₄′ are independently H or alkyl; and
   pharmaceutically acceptable salts thereof.

In some embodiments, the compound is a compound of formula (Ia) and:
R₁ is H;
R₂ and R₃ are each selected from the group consisting of H, —(CR₅R₆)ₙ—O—R₇, —(CR₅R₆)ₙ—Ar—O—C(═O)—R₇, —(CR₅R₆)ₙ—O—C(═O)—R₇, —Ar—C(═O)—O—(CR₅R₆)ₙ—R₇, and —(CR₅R₆)ₙ—O—C(═O)—O—R₇; and
R₄ is selected from the group consisting of —(CR₅R₆)ₙ—O—R₇, —(CR₅R₆)ₙ—Ar—O—C(═O)—R₇, —Ar—C(═O)—O—(CR₅R₆)ₙ—R₇, —(CR₅R₆)ₙ—O—C(═O)—R₇ and —(CR₅R₆)ₙ—O—C(═O)—O—R₇; and
pharmaceutically acceptable salts thereof.

In some embodiments, the compound is a compound of formula (Ia) and:
R₁ is alkyl;
R₂ and R₃ are each independently selected from the group consisting of H, alkyl, —(CR₅R₆)ₙ—O—R₇, —(CR₅R₆)ₙ—Ar—O—C(═O)—R₇, —(CR₅R₆)ₙ—O—[(CR₅R₆)ₙ—O]ₘ—R₇, —(CR₅R₆)ₙ—O—C(═O)—R₇ and —(CR₅R₆)ₙ—O—C(═O)—O—R₇; and
R₄ is selected from the group consisting of —(CR₅R₆)ₙ—O—R₇, —(CR₅R₆)ₙ—Ar—O—C(═O)—R₇, —(CR₅R₆)ₙ—O—[(CR₅R₆)ₙ—O]ₘ—R₇, —(CR₅R₆)ₙ—O—C(═O)—R₇ and —(CR₅R₆)ₙ—O—C(═O)—O—R₇; and
pharmaceutically acceptable salts thereof.

In some embodiments, the compound is a compound of formula (Ia) and:
R₁ is selected from —(CR₅R₆)ₙ—O—C(═O)—R₇ and —(CR₅R₆)ₙ—O—C(═O)—O—R₇; and
R₂ R₃, and R₄ are each independently selected from H, Ar, —(CR₅R₆)ₙ—O—C(═O)—R₇, and —(CR₅R₆)ₙ—O—C(═O)—O—R₇; and
pharmaceutically acceptable salts thereof.

In some embodiments, the compound is a compound of formula (Ia) and:
one of R₁, R₂, R₃, or R₄ is H and the other three are each independently selected from the group consisting of:
—(CR₅R₆)ₙ—O—C(═O)—R₇ and —(CR₅R₆)ₙ—O—C(═O)—O—R₇;
wherein R₅ and R₆ are each independently selected from the group consisting of H, C₁₋₈ straight-chain alkyl, and C₁₋₈ branched-chain alkyl;
R₇ is C₁₋₈ straight-chain alkyl, and C₁₋₈ branched-chain alkyl; and
   pharmaceutically acceptable salts thereof.

In some embodiments, the compound is a compound of formula (Ia) and:
R₂ is H; and
R₁, R₃, and R₄ are each independently selected from the group consisting of:
—(CR₅R₆)ₙ—O—C(═O)—R₇ and —(CR₅R₆)ₙ—O—C(═O)—O—R₇;
wherein R₅ and R₆ are each independently selected from the group consisting of H, C₁₋₈ straight-chain alkyl, and C₁₋₈ branched-chain alkyl;
R₇ is C₁₋₈ straight-chain alkyl or C₁₋₈ branched-chain alkyl; and
   pharmaceutically acceptable salts thereof.

In some embodiments, R₅ and R₆ are each H.

Representative embodiments of 2-PMPA prodrugs are disclosed in:

U.S. Pat. No. 10,544,176 for Prodrugs of Prostate Specific Membrane Antigen (PSMA) Inhibitor, to Slusher et al., issued Jan. 28, 2020;

U.S. Pat. No. 9,988,407 for Prodrugs of Prostate Specific Membrane Antigen (PSMA) Inhibitor, to Slusher et al., issued Jun. 5, 2018;

U.S. Patent Application Publication No. US 2020-0399298 A1 for Prodrugs of Prostate Specific Membrane Antigen (PSMA) Inhibitor, to Slusher et al., published Dec. 24, 2020, each of which is incorporated herein by reference in its entirety.

### C. L-DOPA, D-DOPA, Caffeic acid, and Prodrugs Thereof

In some embodiments, the presently disclosed subject matter provides L-DOPA, D-DOPA, caffeic acid, and prodrugs thereof as GCPII inhibitors. Representative prodrugs of L-DOPA, D-DOPA, and caffeic acid are disclosed in U.S. Provisional Patent Application No. 63/254,344 for DOPA and Caffeic Acid Analogs As Novel GCPII Inhibitors, to Rais et al., filed Oct. 11, 2021, which is incorporated herein by reference in its entirety, including page 9, line 19, through page 14, line 1.

More particularly, the presently disclosed subject matter provides prodrugs of L-DOPA, D-DOPA, and caffeic acid as compounds of formula (II):

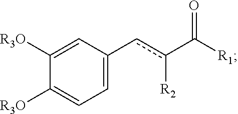

wherein:
[symbol] indicates that the bond can be a single or a double bond;
R₁ is:
—OR₅, wherein R₅ is selected from the group consisting of H, C₁-C₈ alkyl, and —O—(CH₂)ₙ—R₆, wherein n is an integer selected from the group consisting of 0, 1, 2, 3, 4, 5, 6, 7, and 8 and R₆ is substituted or unsubstituted aryl or heteroaryl; or
   —NR₇R₈, wherein R₇ and R₈ are each independently selected from the group consisting of H, C₁-C₄ alkyl, C₃-C₆ cycloalkyl, C₁-C₈ alkoxyl, unsubstituted or substituted aryl or heteroaryl, —(CH₂)ₘ—R₉, wherein R₉ is —OR₁₀ or CHX₂, wherein R₁₀ is H or C₁-C₄ alkyl, and each X is halogen, and —(CH₂)ₘ—CH(NH₂)(COOH), wherein each m is independently an integer selected from the group consisting of 0, 1, 2, 3, 4, 5, 6, 7, and 8;
R₂ is H or —NR₁₁R₁₂, wherein R₁₁ and R₁₂ are each independently selected from the group consisting of H, C₁-C₄ alkyl, and —C(═O)—R₁₃, wherein R₁₃ is C₁-C₄ alkyl or
   —C(NH₂)—(CH₂)ₚ—R₁₄, wherein R₁₄ is C₁-C₄ alkyl or —NR₁₅R₁₆, wherein R₁₅ and R₁₆ are each H or C₁-C₄ alkyl, and p is an integer selected from the group consisting of 0, 1, 2, 3, 4, 5, 6, 7, and 8;
R₃ and R₄ are each independently H or —C(═O)—R₁₇, wherein R₁₇ is C₁-C₈ alkyl or —(CH₂)ₜ—O—C(═O)—O—R₁₈, wherein R₁₈ is C₁-C₈ alkyl, and t is an integer selected from the group consisting of 0, 1, 2, 3, 4, 5, 6, 7, and 8; and
stereoisomers and pharmaceutically acceptable salts thereof.

In certain embodiments, R₁ is —OR₅, and R₅ is selected from the group consisting of H, methyl, ethyl, n-propyl, isopropyl, n-butyl, isobutyl, sec-butyl, tert-butyl, n-pentyl, sec-pentyl, isopentyl, neopentyl, n-hexyl, sec-hexyl, n-heptyl, and n-octyl.

In certain embodiments, R₁ is —OR₅, and R₅ is H or —O—(CH₂)ₙ—R₆, wherein R₆ is substituted or unsubstituted phenyl.

In certain embodiments, R₁ is —NR₇R₈, and R₇ is H or C₁-C₄ alkyl and R₆ is selected from the group consisting of H, C₁-C₄ alkyl, C₃-C₆ cycloalkyl, unsubstituted or substituted phenyl, —(CH₂)ₘ—R₉, wherein R₉ is —OR₁₀ or CHX₂, wherein R₁₀ is H or C₁-C₄ alkyl, and each X is halogen, and —(CH₂)ₘ—CH(NH₂)(COOH), wherein each m is independently an integer selected from the group consisting of 0, 1, 2, 3, 4, 5, 6, 7, and 8.

In certain embodiments, R₂ is —NR₁₁R₁₂, wherein Rn is H and R₁₂ is H or —C(═O)—R₁₃, wherein R₁₃ is C₁-C₄ alkyl or —C(NH₂)—(CH₂)ₚ—R₁₄, wherein R₁₄ is C₁-C₄ alkyl or
—NR₁₅R₁₆, wherein R₁₅ and R₁₆ are each H or C₁-C₄ alkyl, and p is an integer selected from the group consisting of 0, 1, 2, 3, 4, 5, 6, 7, and 8.

In certain embodiments, R₃ and R₄ are each H.

In certain embodiments, if R₁ is —OR₅, then R₅ cannot be H.

In certain embodiments, if R₁ is —OR₅, then R₃, R₄, and R₅ cannot all be H.

In certain embodiments, R₃ and R₄ are each independently selected from the group consisting of —C(═O)—CH₃, —C(═O)—C(CH₃)₃, and —CH₂—O—C(═O)—O—CH(CH₃)₂.

In particular embodiments, the compound of formula (II) is selected from the group consisting of:

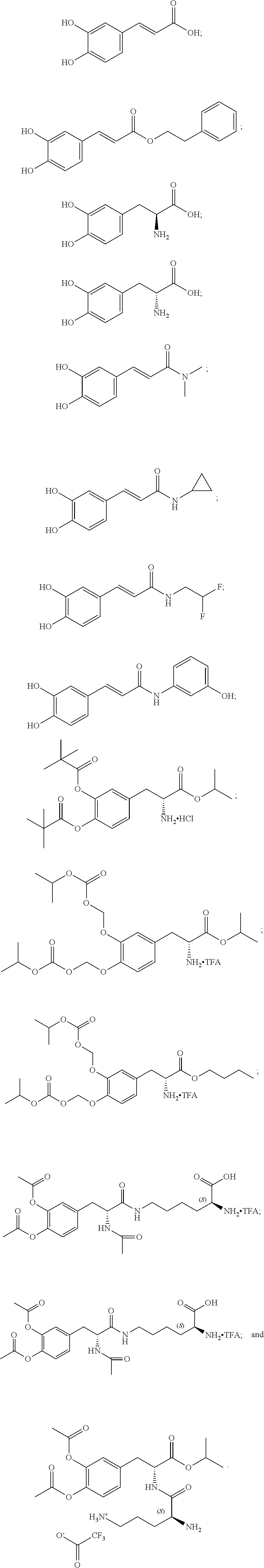

Representative GCPII inhibitor and their prodrugs having a catechol scaffold are provided in Table 2.

TABLE 2
Catechol Scaffolds for GCPII Inhibition

| Compound | Structures | IC₅₀ |
|---|---|---|
| Caffeic acid | 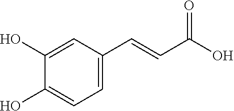 [SMILES: O=C(O)/C=C/c1ccc(O)c(O)c1] | 100 nm |
| CAPE | 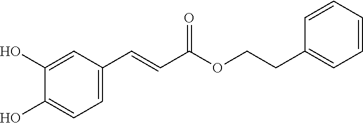 [SMILES: O=C(/C=C/c1ccc(O)c(O)c1)OCCc1ccccc1] | — |
| JHU-5144 | 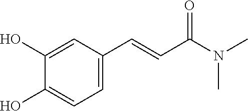 | — |
| JHU-5145 | 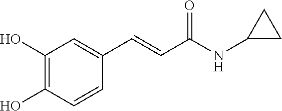 [SMILES: O=C(/C=C/c1ccc(O)c(O)c1)NC1CC1] | — |
| JHU-5146 | 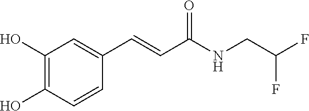 [SMILES: O=C(/C=C/c1ccc(O)c(O)c1)NCC(F)F] | — |
| JHU-5147 | 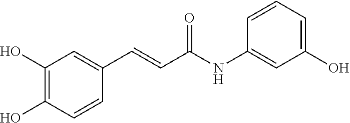 [SMILES: O=C(/C=C/c1ccc(O)c(O)c1)Nc1cccc(O)c1] | — |
| TBD | 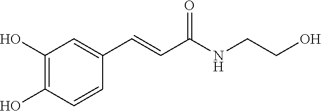 [SMILES: O=C(/C=C/c1ccc(O)c(O)c1)NCCO] | — |
| TBD | 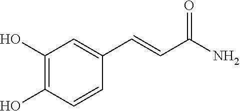 [SMILES: NC(=O)/C=C/c1ccc(O)c(O)c1] | — |
| L-DOPA | 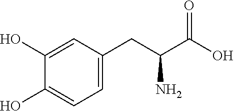 [SMILES: N[C@@H](Cc1ccc(O)c(O)c1)C(=O)O] | 400 nM |
| D-DOPA | 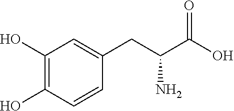 [SMILES: N[C@H](Cc1ccc(O)c(O)c1)C(=O)O] | 200 nM |
| Prodrug-1 | 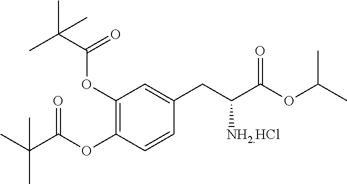 | — |
| Prodrug-2 | 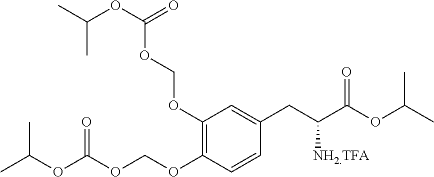 | — |
| Prodrug-3 | 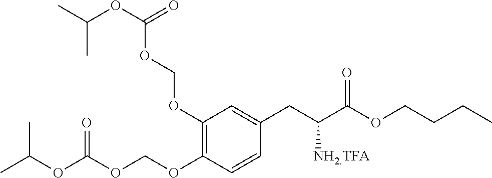 | — |
| Prodrug-4 | 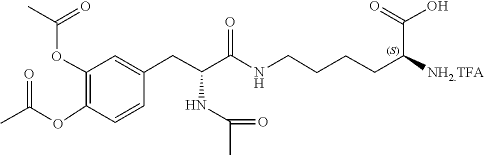 | — |
| Prodrug-5 | 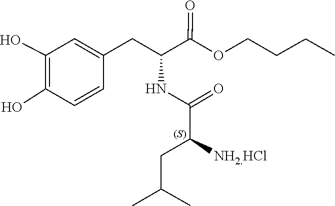 | — |
| Prodrug-7 | 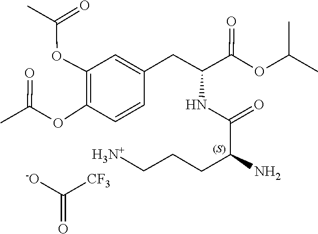 | — |

### D. Prodrugs of Hydroxamate-Based GCPII Inhibitors

Representative prodrugs of hydroxamate-based GCPII inhibitors are disclosed in International PCT Patent Application Publication No. WO2018094334 for Prodrugs of Hydroxamate-Based GCPII Inhibitors, to Slusher et al., published May 24, 2018, which is incorporated herein by reference in its entirety, in particular, page 6, line 31, through page 17, line 1.

In some embodiments, the presently disclosed subject matter provides a compound of formula (III):

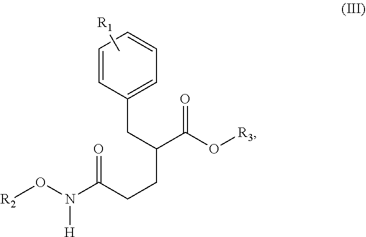

wherein: R₁ is selected from the group consisting of —C(═O)—O—R₄ and —Ar—C(═O)—O—R₄; R₂ is selected from the group consisting of substituted and unsubstituted C₁-C₆ alkyl, substituted and unsubstituted C₃-C₈ cycloalkyl, substituted and unsubstituted C₆-C₁₂ aryl, substituted and unsubstituted C₆-C₁₂ heteroaryl, —(CR₅R₆)ₙ—R₇, —C(═O)—O—R₇, —C(═O)—R₇, —C(═O)—NR₇R₈, —(CR₅R₆)ₙ—O—C(═O)—O—R₇, —(CR₅R₆)ₙ—Ar—O—C(═O)—R₇; R₃ is selected from the group consisting of H, substituted and unsubstituted C₁-C₆ alkyl, substituted and unsubstituted C₃-C₁₂ cycloalkyl, substituted and unsubstituted C₆-C₁₂ aryl, substituted and unsubstituted C₅-C₁₂ heteroaryl; R₄ is selected from the group consisting of H, substituted and unsubstituted C₁-C₆ alkyl, substituted and unsubstituted C₃-C₁₂ cycloalkyl, substituted and unsubstituted C₆-C₁₂ aryl, substituted and unsubstituted C₅-C₁₂ heteroaryl, —(CR₅R₆)ₙ—O—C(═O)—O—R₉, and —(CR₅R₆)ₙ—Ar—O—C(═O)—R₉; each R₅ and R₆ is independently selected from the group consisting of H, C₁-C₁₀ alkyl, and C₆-C₁₂ aralkyl; R₇ is selected from the group consisting of H, and substituted and unsubstituted C₁-C₁₀ alkyl, substituted and unsubstituted C₁-C₁₀ heteroalkyl, substituted and unsubstituted C₃-C₁₆ cycloalkyl, substituted and unsubstituted C₃-C₁₂ cycloheteroalkyl, substituted and unsubstituted C₃-C₁₂ cycloheteroalkenyl, substituted and unsubstituted C₆-C₁₂ aryl, substituted and unsubstituted C₆-C₁₂ heteroaryl, and substituted and unsubstituted C₆-C₁₂ aralkyl; R₈ is selected from the group consisting of H, and substituted and unsubstituted C₁-C₆ alkyl; R₉ is selected from the group consisting of H, and substituted and unsubstituted C₁-C₆ alkyl; n is an integer selected from the group consisting of 0, 1, 2, 3, 4, 5, and 6; Ar is selected from the group consisting of substituted and unsubstituted C₆-C₁₂ aryl, and substituted and unsubstituted C₆-C₁₂ heteroaryl; and stereoisomers and pharmaceutically acceptable salts thereof.

In some embodiments, the compound of formula (III) is selected from the group consisting of:

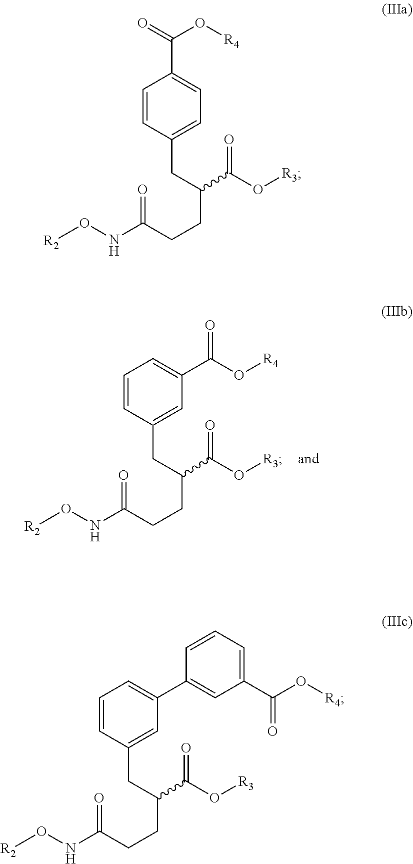

wherein R₂, R₃, and R₄, are as defined hereinabove; and stereoisomers and pharmaceutically acceptable salts thereof.

In some embodiments, R₂ is as defined hereinabove; R₃ is selected from the group consisting of H and substituted and unsubstituted C₁-C₆ alkyl; R₄ is selected from the group consisting of H, substituted and unsubstituted C₁-C₆ alkyl, —(CR₅R₆)ₙ—Ar—O—C(═O)—R₉, and —(CR₅R₆)ₙ—O—C(═O)—O—R₉; n is 1; R₅ and R₆ are H; Ar is phenyl; R₉ is selected from the group consisting of substituted C₁-C₃ alkyl, and unsubstituted C₁-C₃ alkyl; and stereoisomers and pharmaceutically acceptable salts thereof.

In some embodiments R₂ is —(CR₅R₆)ₙ—Ar—O—C(═O)—R₇, n is 1, Ar is phenyl, and R₇ is substituted or unsubstituted C₁-C₆ alkyl.

In particular embodiments, the compound of formula (III) is selected from the group consisting of:

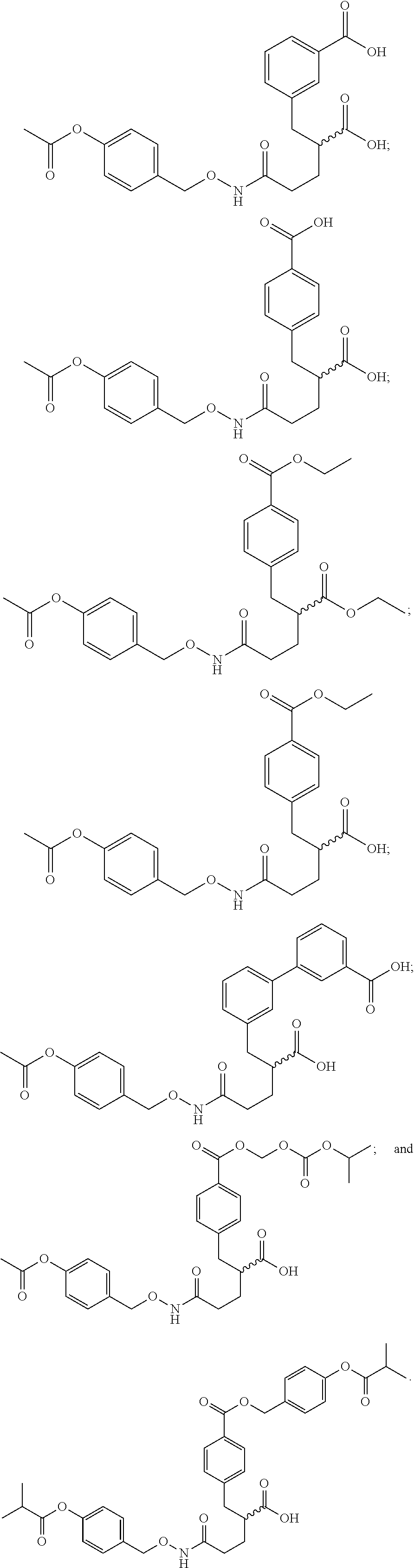

In other embodiments, R₂ is —(CR₅R₆)ₙ—R₇, n is 1, and R₇ is substituted C₃-C₁₂ cycloheteroalkenyl. In particular embodiments, the compound of formula (III) is selected from the group consisting of:

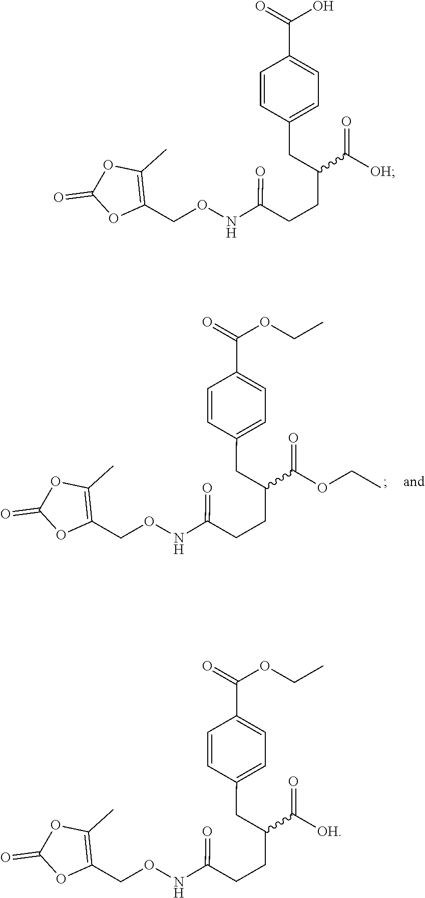 [SMILES: Cc1oc(=O)oc1CONC(=O)CCC(Cc1ccc(C(=O)O)cc1)C(=O)O ; CCOC(=O)c1ccc(CC(CCC(=O)NOCc2oc(=O)oc2C)C(=O)OCC)cc1 ; CCOC(=O)c1ccc(CC(CCC(=O)NOCc2oc(=O)oc2C)C(=O)O)cc1]

In some embodiments R₂ is —C(═O)—R₇, and R₇ is unsubstituted C₁-C₆ alkyl, substituted C₁-C₆ alkyl, unsubstituted C₆-C₁₂ aryl, or unsubstituted C₆-C₁₂ aralkyl. In particular embodiments, the compound of formula (III) is selected from the group consisting of:

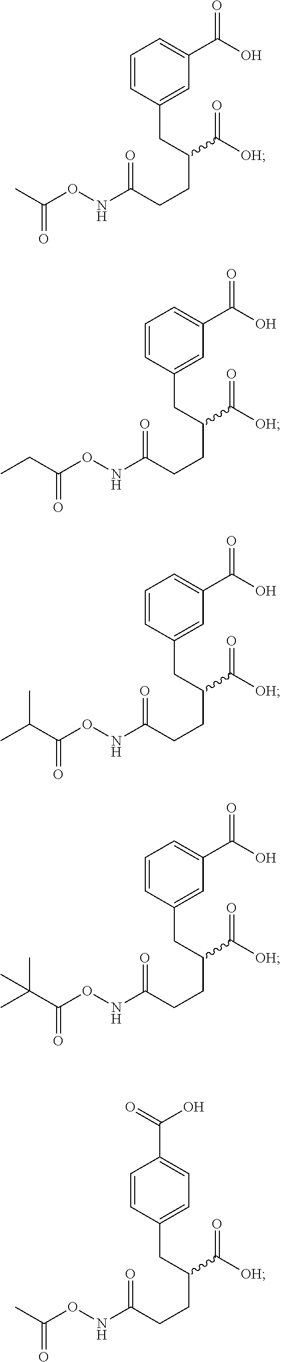 [SMILES: CC(=O)ONC(=O)CCC(Cc1cccc(C(=O)O)c1)C(=O)O ; CCC(=O)ONC(=O)CCC(Cc1cccc(C(=O)O)c1)C(=O)O ; CC(C)C(=O)ONC(=O)CCC(Cc1cccc(C(=O)O)c1)C(=O)O ; CC(C)(C)C(=O)ONC(=O)CCC(Cc1cccc(C(=O)O)c1)C(=O)O ; CC(=O)ONC(=O)CCC(Cc1ccc(C(=O)O)cc1)C(=O)O]

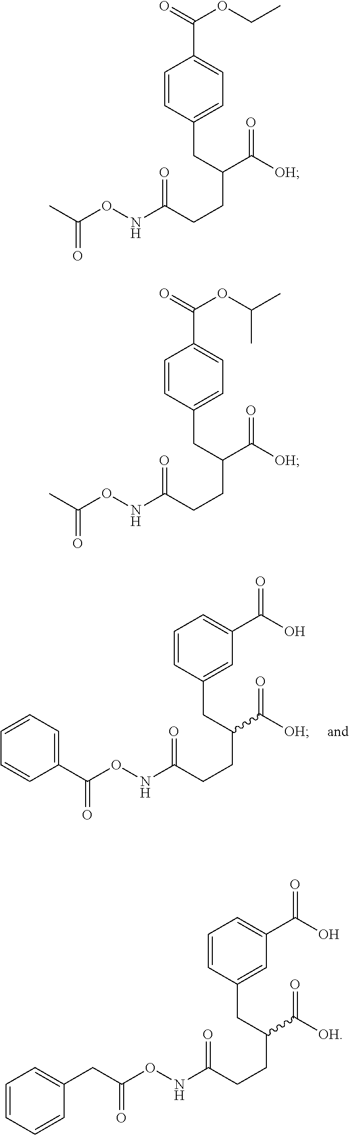

In some embodiments, R₂ is —C(═O)—O—R₇, and R₇ is unsubstituted C₁-C₆ alkyl. In particular embodiments, the compound of formula (III) is:

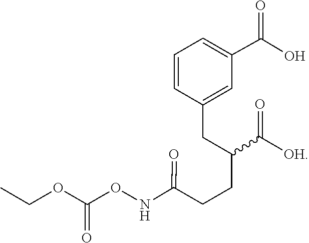 [SMILES: CCOC(=O)ONC(=O)CCC(Cc1cccc(C(=O)O)c1)C(=O)O]

In some embodiments, R₂ is —C(═O)—NR₇R₈, R₇ is substituted C₁-C₆ alkyl, or substituted C₃-C₁₆ cycloalkyl, and R₈ is H. In particular embodiments, the compound of formula (III) is selected from the group consisting of:

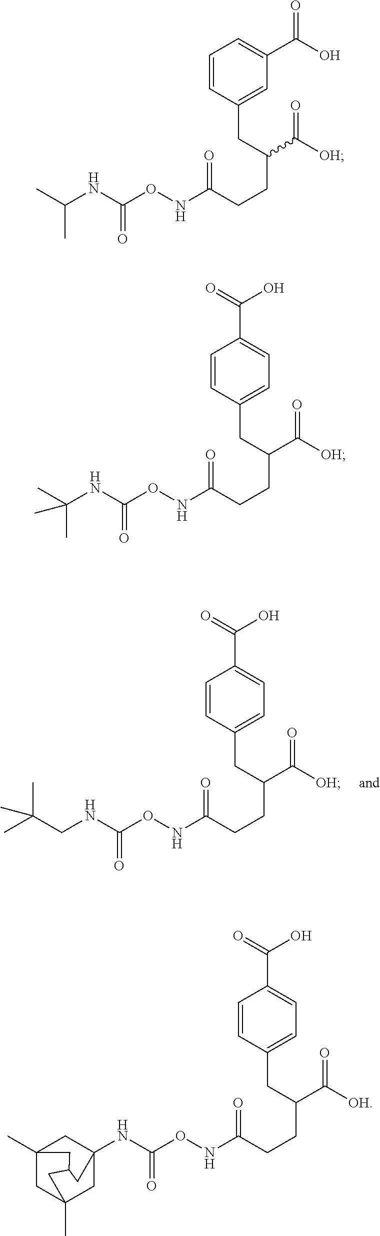 [SMILES: CC(C)NC(=O)ONC(=O)CCC(Cc1cccc(C(=O)O)c1)C(=O)O ; CC(C)(C)NC(=O)ONC(=O)CCC(Cc1ccc(C(=O)O)cc1)C(=O)O ; CC(C)(C)CNC(=O)ONC(=O)CCC(Cc1ccc(C(=O)O)cc1)C(=O)O ; CC12CC3CC(C)(C1)CC(NC(=O)ONC(=O)CCC(Cc1ccc(C(=O)O)cc1)C(=O)O)(C3)C2]

Representative structures of prodrugs of hydroxamate-based GCPII inhibitors are provided in Table 3.

TABLE 3
STRUCTURES OF REPRESENTATIVE PRODRUGS OF
HYDROXAMATE-BASED GCPII INHIBITORS

| IOCB No./ | | |
|---|---|---|
| Compound No. | Structure | MW |
| JV2677 32 | 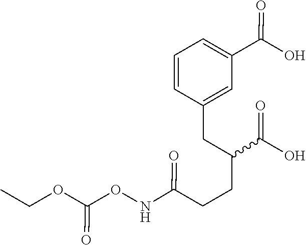 [SMILES: CCOC(=O)ONC(=O)CCC(Cc1cccc(C(=O)O)c1)C(=O)O] | 353.33 |
| JV2678 | 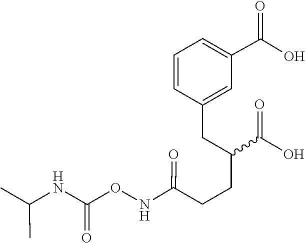 [SMILES: CC(C)NC(=O)ONC(=O)CCC(Cc1cccc(C(=O)O)c1)C(=O)O] | 366.37 |
| JV2680 | 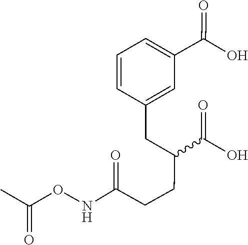 [SMILES: CC(=O)ONC(=O)CCC(Cc1cccc(C(=O)O)c1)C(=O)O] | 323.30 |
| JV2719 | 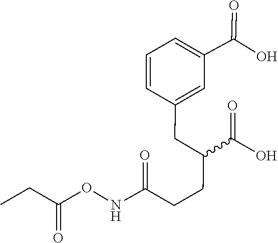 [SMILES: CCC(=O)ONC(=O)CCC(Cc1cccc(C(=O)O)c1)C(=O)O] | 337.33 |
| JV2720 | 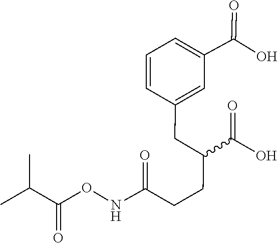 [SMILES: CC(C)C(=O)ONC(=O)CCC(Cc1cccc(C(=O)O)c1)C(=O)O] | 351.36 |
| JV2721 | 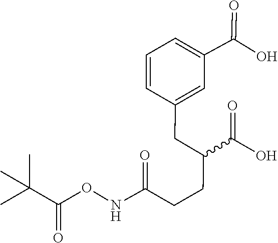 [SMILES: CC(C)(C)C(=O)ONC(=O)CCC(Cc1cccc(C(=O)O)c1)C(=O)O] | 365.38 |
| JV2722 | 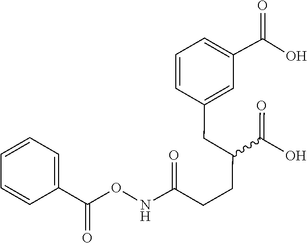 [SMILES: O=C(CCC(Cc1cccc(C(=O)O)c1)C(=O)O)NOC(=O)c1ccccc1] | 385.37 |
| JV2723 | 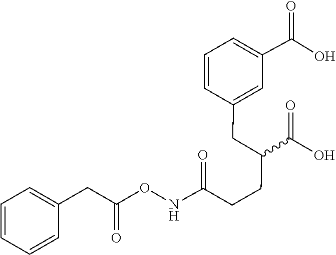 [SMILES: O=C(CCC(Cc1cccc(C(=O)O)c1)C(=O)O)NOC(=O)Cc1ccccc1] | 399.40 |
| JV2837 | 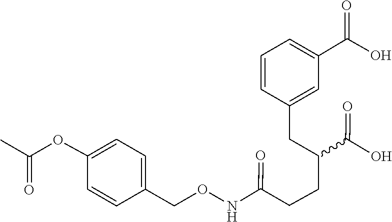 [SMILES: CC(=O)Oc1ccc(CONC(=O)CCC(Cc2cccc(C(=O)O)c2)C(=O)O)cc1] | 429.43 |
| JV2855 | 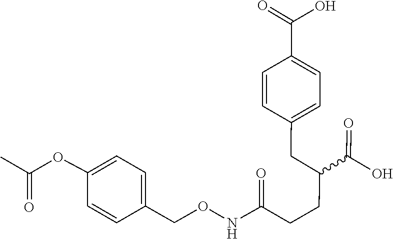 [SMILES: CC(=O)Oc1ccc(CONC(=O)CCC(Cc2ccc(C(=O)O)cc2)C(=O)O)cc1] | 429.43 |
| JV2925 | 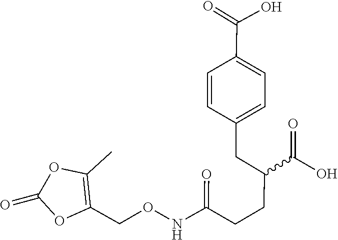 [SMILES: Cc1oc(=O)oc1CONC(=O)CCC(Cc1ccc(C(=O)O)cc1)C(=O)O] | 393.35 |
| JV2928 | 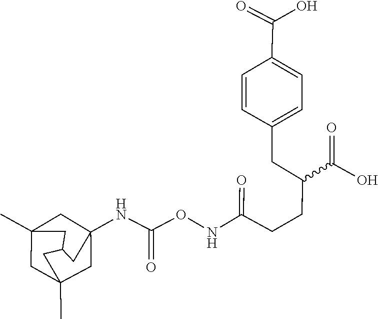 [SMILES: CC12CC3CC(C)(C1)CC(NC(=O)ONC(=O)CCC(Cc1ccc(C(=O)O)cc1)C(=O)O)(C3)C2] | 486.57 |
| JV2946 | 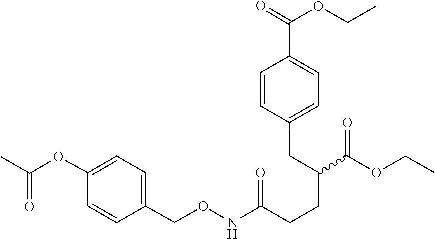 [SMILES: CCOC(=O)c1ccc(CC(CCC(=O)NOCc2ccc(OC(C)=O)cc2)C(=O)OCC)cc1] | 485.53 |
| JV2956 | 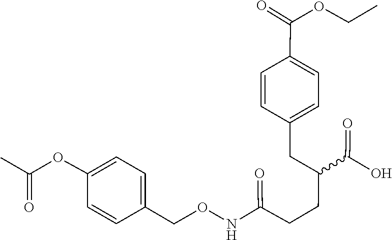 [SMILES: CCOC(=O)c1ccc(CC(CCC(=O)NOCc2ccc(OC(C)=O)cc2)C(=O)O)cc1] | 457.48 |
| JV2947 | 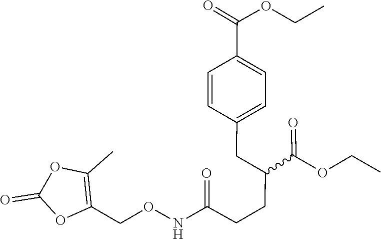 [SMILES: CCOC(=O)c1ccc(CC(CCC(=O)NOCc2oc(=O)oc2C)C(=O)OCC)cc1] | 449.46 |
| JV2957 | 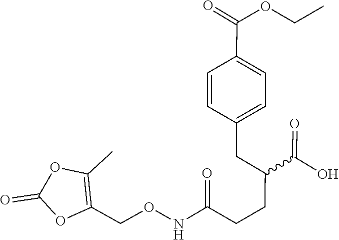 [SMILES: CCOC(=O)c1ccc(CC(CCC(=O)NOCc2oc(=O)oc2C)C(=O)O)cc1] | 421.40 |
| JV3055 | 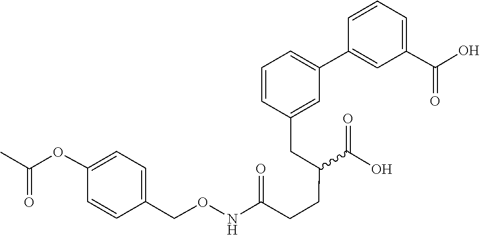 [SMILES: CC(=O)Oc1ccc(CONC(=O)CCC(Cc2cccc(-c3cccc(C(=O)O)c3)c2)C(=O)O)cc1] | 505.52 |
| TT271115 | 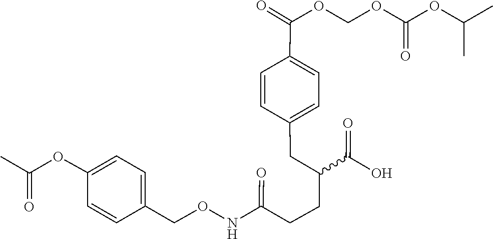 [SMILES: CC(=O)Oc1ccc(CONC(=O)CCC(Cc2ccc(C(=O)OCOC(=O)OC(C)C)cc2)C(=O)O)cc1] | 545.54 |
| TT200216 | 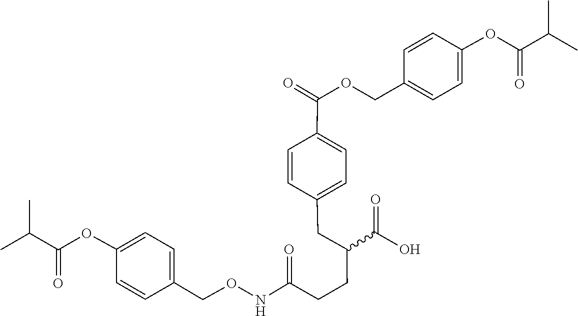 [SMILES: CC(C)C(=O)Oc1ccc(CONC(=O)CCC(Cc2ccc(C(=O)OCc3ccc(OC(=O)C(C)C)cc3)cc2)C(=O)O)cc1] | 633.69 |
| TT171214 | 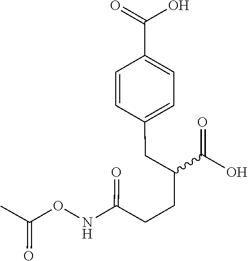 [SMILES: CC(=O)ONC(=O)CCC(Cc1ccc(C(=O)O)cc1)C(=O)O] | 323.30 |
| JV3113 | 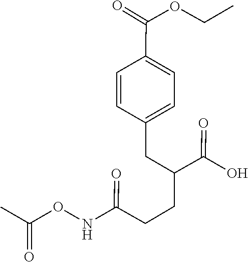 [SMILES: CCOC(=O)c1ccc(CC(CCC(=O)NOC(C)=O)C(=O)O)cc1] | 351.36 |
| JV3138 | 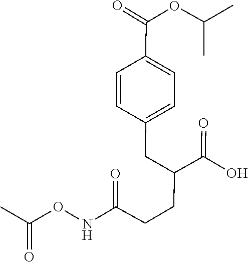 [SMILES: CC(=O)ONC(=O)CCC(Cc1ccc(C(=O)OC(C)C)cc1)C(=O)O] | 365.38 |
| JV3126 | 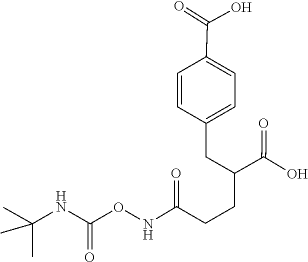 [SMILES: CC(C)(C)NC(=O)ONC(=O)CCC(Cc1ccc(C(=O)O)cc1)C(=O)O] | 380.40 |
| JV3127 | 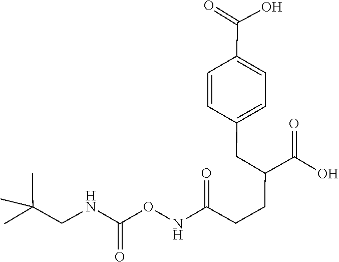 [SMILES: CC(C)(C)CNC(=O)ONC(=O)CCC(Cc1ccc(C(=O)O)cc1)C(=O)O] | 394.42 |

Representative prodrugs of hydroxamate-based GCPII inhibitors are disclosed in:
U.S. Pat. No. 11,059,775 for Prodrug compositions and utility of hydroxamate-based GCPII inhibitors, to Slusher et al., issued Jul. 13, 2021; and
U.S. Patent Application Publication No. US 2021-0355079 A1 for Prodrug compositions and utility of hydroxamate-based GCPII inhibitors, to Slusher et al., published Nov. 18, 2021, each of which is incorporated herein by reference in its entirety.

### E. Dendrimer Conjugates of 2-PMPA

In some embodiments, the presently disclosed subject matter provides dendrimer conjugates of 2-PMPA and their use for treating age-related sarcopenia and/or enhancing longevity. In some embodiments, the dendrimers are in the form of dendrimer nanoparticles comprising poly(amidoamine) (PAMAM) hydroxyl-terminated dendrimers covalently linked to 2-PMPA. Representative dendrimer compositions suitable for use with the presently disclosed methods for treating age-related sarcopenia and/or enhancing longevity are disclosed in International PCT Patent Application Publication No. WO2016025745 for Dendrimer Compositions and use in Treatment of Neurological and CNS Disorders, to Rangaramanujam et al., published Feb. 18, 2016, which is incorporated herein in its entirety.

In particular embodiments, the dendrimer nanoparticles include one or more ethylene diamine-core PAMAM hydroxyl-terminated generation-4 through generation-10 (e.g., ≥G4-OH) dendrimers covalently linked to 2-PMPA.

As used herein, the term “dendrimer” includes, but is not limited to, a molecular architecture having an interior core, interior layers (or “generations”) of repeating units regularly attached to the interior core, and an exterior surface of terminal groups attached to the outermost generation. Dendrimers suitable for use with the presently disclosed methods include, but are not limited to, polyamidoamine (PAMAM), polypropyiamine (POPAM), poly(propylene imine) (PPI), polyethylenimine, polylysine, polyester, iptycene, aliphatic poly(ether), and/or aromatic polyether dendrimers. Each dendrimer of the dendrimer complex may be of similar or different chemical nature than the other dendrimers (e.g., the first dendrimer may include a PAMAM dendrimer, while the second dendrimer may comprise a POPAM dendrimer). In some embodiments, the first or second dendrimer may further include an additional agent. In some embodiments, a multiarm PEG polymer can include a polyethylene glycol having at least two branches bearing sulfhydryl or thiopyridine terminal groups; however, embodiments disclosed herein are not limited to this class and PEG polymers hearing other terminal groups, such as succinimidyl or maleimide terminal groups, can be used. In particular embodiments, PEG polymers in the molecular weight 10 kDa to 80 kDa can be used.

In certain embodiments, the dendrimer complex can include multiple dendrimers. For example, the dendrimer complex can include a third dendrimer; wherein the third-dendrimer is complexed with at least one other dendrimer. Further, a third agent can be complexed with the third dendrimer. In another embodiment, the first and second dendrimers are each complexed to a third dendrimer, wherein the first and second dendrimers are PAMAM dendrimers and the third dendrimer is a POPAM dendrimer. Additional dendrimers also can be incorporated. When multiple dendrimers are used, multiple agents also can be incorporated. This characteristic is not limited by the number of dendrimers complexed to one another.

As used herein, the term “PAMAM dendrimer” refers to a poly(amidoamine) dendrimer, which may contain different cores, with amidoamine building blocks. The method for making them is known to those of skill In the art and generally, involves a two-step iterative reaction sequence that produces concentric shells (i.e., “generations”) of dendritic β-alanine units around a central interior core. This PAMAM core-shell architecture grows linearly in diameter as a function of added shells (generations). Meanwhile, the surface groups amplify exponentially at each generation according to dendritic-branching mathematics. Such dendrimers are available in generations G0-G10 with 5 different core types and 10 functional surface groups.

In certain embodiments, the PAMAM dendrimers can have carboxylic, amine and hydroxyl terminal groups and can be any generation of dendrimers including, but not limited to, generation 1 PAMAM dendrimers, generation 2 PAMAM dendrimers, generation 3 PAMAM dendrimers, generation 4 PAMAM dendrimers, generation 5 PAMAM dendrimers, generation 6 PAMAM dendrimers, generation 7 PAMAM dendrimers, generation 8 PAMAM dendrimers, generation 9 PAMAM dendrimers, or generation 10 PAMAM dendrimers. In particular embodiments, the PAMAM dendrimers can be generation 4 dendrimers, or more, with hydroxyl groups attached to their functional surface groups.

Representative dendrimers suitable for use with the presently disclosed methods are disclosed in International PCT Patent Application Publication No. WO2009/046446 for Dendrimers for Sustained Release of Compounds, to Kannan et al., published Apr. 9, 2009, which is incorporated herein by reference in its entirety.

Dendrimer complexes can be formed by covalently bonding or otherwise attaching, e.g., via intermolecularly dispersion or encapsulation, a therapeutically active agent, e.g., 2-PMPA, to a dendrimer or multiarm PEG. The attachment can occur via an appropriate spacer that provides a disulfide bridge between the agent and the dendrimer. The dendrimer complexes are capable of rapid release of the agent in vivo by thiol exchange reactions, under the reduced conditions found in a body.

The term “spacers” as used herein is intended to include compositions used for linking a therapeutically active agent to the dendrimer. The spacer can be either a single chemical entity or two or more chemical entities linked together to bridge the polymer and the therapeutic agent or imaging agent. The spacers can include any small chemical entity, peptide or polymers having sulfhydryl, thiopyridine, succinimidyl, maleimide, vinylsulfone, and carbonate terminal groups.

In certain embodiments, the spacer can comprise thiopyridine terminated compounds including, but not limited to, dithiodipyridine, N-succinimidyl 3-(2-pyridyldithio)-propionate (SPDP), succinimidyl 6-(3-[2-pyridyldithio]-propionamido)hexanoate LC-SPDP, or Sulfo-LC-SPDP. The spacer also can include peptides wherein the peptides are linear or cyclic having sulfhydryl groups, such as glutathione, homocysteine, cysteine and its derivatives, arg-gly-asp-cys (RGDC), cyclo(Arg-Gly-Asp-d-Phe-Cys) (c(RGDfC)), cyclo(Arg-Gly-Asp-D-Tyr-Cys), cyclo(Arg-Ala-Asp-d-Tyr-Cys). The spacer can be a mercapto acid derivative such as 3-mercapto propionic acid, mercapto acetic acid, 4-mercapto butyric acid, thiolan-2-one, 6-mercaptohexanoic acid, 5-mercapto valeric acid and other mercapto derivatives such as 2-mercaptoethanol and 2-mercaptoethylamine. The spacer can be thiosalicyclic acid and its derivatives including (4-succinimidyloxycarbonyl-methyl-α-2-pyridylthio)toluene and (3-[2-pyridithio]propionyl hydrazide. The spacer can have maleimide terminal groups wherein the spacer comprises polymer or small chemical entity, such as bis-maleimido diethylene glycol and bis-maleimido triethylene glycol, bismaleimidoethane, bismaleimidohexane. The spacer can comprise a vinylsulfone, such as 1,6-hexane-bis-vinylsulfone. The spacer can comprise thioglycosides, such as thioglucose. The spacer can be a reduced protein, such as bovine serum albumin and human serum albumin, or any thiol terminated compound capable of forming disulfide bonds. The spacer can include polyethylene glycol having maleimide, succinimidyl and thiol terminal groups.

As provided hereinabove, in some embodiments, the presently disclosed subject matter provides a method for treating age-related sarcopenia and/or enhancing longevity,

As used herein, the term “treating” can include reversing, alleviating, inhibiting the progression of, preventing or reducing the likelihood of the disease, disorder, or condition to which such term applies, or one or more symptoms or manifestations of such disease, disorder or condition. Preventing refers to causing a disease, disorder, condition, or symptom or manifestation of such, or worsening of the severity of such, not to occur. Accordingly, the presently disclosed compounds can be administered prophylactically to prevent or reduce the incidence or recurrence of the disease, disorder, or condition.

The “subject” treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. The term “subject” also refers to an organism, tissue, cell, or collection of cells from a subject.

In general, the “effective amount” of an active agent or drug delivery device refers to the amount necessary to elicit the desired biological response. As will be appreciated by those of ordinary skill in this art, the effective amount of an agent or device may vary depending on such factors as the desired biological endpoint, the agent to be delivered, the makeup of the pharmaceutical composition, the target tissue, and the like.

The term “combination” is used in its broadest sense and means that a subject is administered at least two agents, more particularly a compound described herein and at least one other therapeutic agent. More particularly, the term “in combination” refers to the concomitant administration of two (or more) active agents for the treatment of a, e.g., single disease state. As used herein, the active agents may be combined and administered in a single dosage form, may be administered as separate dosage forms at the same time, or may be administered as separate dosage forms that are administered alternately or sequentially on the same or separate days. In one embodiment of the presently disclosed subject matter, the active agents are combined and administered in a single dosage form. In another embodiment, the active agents are administered in separate dosage forms (e.g., wherein it is desirable to vary the amount of one but not the other). The single dosage form may include additional active agents for the treatment of the disease state.

Further, the compounds described herein can be administered alone or in combination with adjuvants that enhance stability of the compounds, alone or in combination with one or more therapeutic agents, facilitate administration of pharmaceutical compositions containing them in certain embodiments, provide increased dissolution or dispersion, increase inhibitory activity, provide adjunct therapy, and the like, including other active ingredients. Advantageously, such combination therapies utilize lower dosages of the conventional therapeutics, thus avoiding possible toxicity and adverse side effects incurred when those agents are used as monotherapies.

The timing of administration of a compound described herein and at least one additional therapeutic agent can be varied so long as the beneficial effects of the combination of these agents are achieved. Accordingly, the phrase “in combination with” refers to the administration of a compound described herein and at least one additional therapeutic agent either simultaneously, sequentially, or a combination thereof. Therefore, a subject administered a combination of a compound described herein and at least one additional therapeutic agent can receive a compound and at least one additional therapeutic agent at the same time (i.e., simultaneously) or at different times (i.e., sequentially, in either order, on the same day or on different days), so long as the effect of the combination of both agents is achieved in the subject.

When administered sequentially, the agents can be administered within 1, 5, 10, 30, 60, 120, 180, 240 minutes or longer of one another. In other embodiments, agents administered sequentially, can be administered within 1, 5, 10, 15, 20 or more days of one another. Where the compound described herein and at least one additional therapeutic agent are administered simultaneously, they can be administered to the subject as separate pharmaceutical compositions, each comprising either a compound or at least one additional therapeutic agent, or they can be administered to a subject as a single pharmaceutical composition comprising both agents.

When administered in combination, the effective concentration of each of the agents to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times.

In some embodiments, when administered in combination, the two or more agents can have a synergistic effect. As used herein, the terms “synergy,” “synergistic,” “synergistically” and derivations thereof, such as in a “synergistic effect” or a “synergistic combination” or a “synergistic composition” refer to circumstances under which the biological activity of a combination of a compound described herein and at least one additional therapeutic agent is greater than the sum of the biological activities of the respective agents when administered individually.

Synergy can be expressed in terms of a “Synergy Index (SI),” which generally can be determined by the method described by F. C. Kull et al., Applied Microbiology 9, 538 (1961), from the ratio determined by:

$${Q}_{a} / {Q}_{A} + {Q}_{b} / {Q}_{B} = Synergy Index ( SI )$$

wherein:
Q_{A} is the concentration of a component A, acting alone, which produced an end point in relation to component A;
Qₐ is the concentration of component A, in a mixture, which produced an end point;
Q_{B} is the concentration of a component B, acting alone, which produced an end point in relation to component B; and
Q_{b} is the concentration of component B, in a mixture, which produced an end point.

Generally, when the sum of Qₐ/Q_{A} and Q_{b}/Q_{B} is greater than one, antagonism is indicated. When the sum is equal to one, additivity is indicated. When the sum is less than one, synergism is demonstrated. The lower the SI, the greater the synergy shown by that particular mixture. Thus, a “synergistic combination” has an activity higher that what can be expected based on the observed activities of the individual components when used alone. Further, a “synergistically effective amount” of a component refers to the amount of the component necessary to elicit a synergistic effect in, for example, another therapeutic agent present in the composition.

### F. Pharmaceutical Compositions and Administration

In another aspect, the present disclosure provides a pharmaceutical composition including one compound described herein alone or in combination with one or more additional therapeutic agents in admixture with a pharmaceutically acceptable excipient. One of skill in the art will recognize that the pharmaceutical compositions include the pharmaceutically acceptable salts of the compounds described above. Pharmaceutically acceptable salts are generally well known to those of ordinary skill in the art, and include salts of active compounds which are prepared with relatively nontoxic acids or bases, depending on the particular substituent moieties found on the compounds described herein. When compounds of the present disclosure contain relatively acidic functionalities, base addition salts can be obtained by contacting the neutral form of such compounds with a sufficient amount of the desired base, either neat or in a suitable inert solvent or by ion exchange, whereby one basic counterion (base) in an ionic complex is substituted for another. Examples of pharmaceutically acceptable base addition salts include sodium, potassium, calcium, ammonium, organic amino, or magnesium salt, or a similar salt.

When compounds of the present disclosure contain relatively basic functionalities, acid addition salts can be obtained by contacting the neutral form of such compounds with a sufficient amount of the desired acid, either neat or in a suitable inert solvent or by ion exchange, whereby one acidic counterion (acid) in an ionic complex is substituted for another. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-toluenesulfonic, citric, tartaric, methanesulfonic, trifluoroacetic acid (TFA), and the like. Also included are salts of amino acids such as arginate and the like, and salts of organic acids like glucuronic or galactunoric acids and the like (see, for example, Berge et al, “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Accordingly, pharmaceutically acceptable salts suitable for use with the presently disclosed subject matter include, by way of example but not limitation, acetate, benzenesulfonate, benzoate, bicarbonate, bitartrate, bromide, calcium edetate, camsylate, carbonate, citrate, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, mucate, napsylate, nitrate, pamoate (embonate), pantothenate, phosphate/diphosphate, polygalacturonate, salicylate, stearate, subacetate, succinate, sulfate, tannate, tartrate, or teoclate. Other pharmaceutically acceptable salts may be found in, for example, Remington: The Science and Practice of Pharmacy (20^{th} ed.) Lippincott, Williams & Wilkins (2000). In therapeutic and/or diagnostic applications, the compounds of the disclosure can be formulated for a variety of modes of administration, including systemic and topical or localized administration. Techniques and formulations generally may be found in Remington: The Science and Practice of Pharmacy (20^{th} ed.) Lippincott, Williams & Wilkins (2000). Depending on the specific conditions being treated, such agents may be formulated into liquid or solid dosage forms and administered systemically or locally.

The agents may be delivered, for example, in a timed- or sustained-slow release form as is known to those skilled in the art. Techniques for formulation and administration may be found in Remington: The Science and Practice of Pharmacy (20^{th} ed.) Lippincott, Williams & Wilkins (2000). Suitable routes may include oral, buccal, by inhalation spray, sublingual, rectal, transdermal, vaginal, transmucosal, nasal or intestinal administration; parenteral delivery, including intramuscular, subcutaneous, intramedullary injections, as well as intrathecal, direct intraventricular, intravenous, intra-articullar, intra-sternal, intra-synovial, intra-hepatic, intralesional, intracranial, intraperitoneal, intranasal, or intraocular injections or other modes of delivery.

For injection, the agents of the disclosure may be formulated and diluted in aqueous solutions, such as in physiologically compatible buffers such as Hank's solution, Ringer's solution, or physiological saline buffer. For such transmucosal administration, penetrants appropriate to the barrier to be permeated are used in the formulation. Such penetrants are generally known in the art.

Use of pharmaceutically acceptable inert carriers to formulate the compounds herein disclosed for the practice of the disclosure into dosages suitable for systemic administration is within the scope of the disclosure. With proper choice of carrier and suitable manufacturing practice, the compositions of the present disclosure, in particular, those formulated as solutions, may be administered parenterally, such as by intravenous injection. The compounds can be formulated readily using pharmaceutically acceptable carriers well known in the art into dosages suitable for oral administration. Such carriers enable the compounds of the disclosure to be formulated as tablets, pills, capsules, liquids, gels, syrups, slurries, suspensions and the like, for oral ingestion by a subject (e.g., patient) to be treated.

For nasal or inhalation delivery, the agents of the disclosure also may be formulated by methods known to those of skill in the art, and may include, for example, but not limited to, examples of solubilizing, diluting, or dispersing substances, such as saline; preservatives, such as benzyl alcohol; absorption promoters; and fluorocarbons.

In particular embodiments, the compound of formulae (I-III) is administered intranasally in a form selected from the group consisting of a nasal spray, a nasal drop, a powder, a granule, a cachet, a tablet, an aerosol, a paste, a cream, a gel, an ointment, a salve, a foam, a paste, a lotion, a cream, an oil suspension, an emulsion, a solution, a patch, and a stick.

As used herein, the term administrating via an “intranasal route” refers to administering by way of the nasal structures. It has been found that the presently disclosed small molecule GCP-II inhibitors are much more effective at penetrating the brain and peripheral nervous system when administered intranasally.

As used herein, the term “peripheral nervous system” includes the part of the nervous system comprising the nerves and ganglia on the outside of the brain and spinal cord. The peripheral nervous system connects the central nervous system to the limbs and organs and acts as a communication relay between the brain and the extremities. The presently disclosed small molecule GCP-II inhibitors can access the peripheral nervous system through the blood.

Intranasal administration generally allows the active agent to bypass first pass metabolism, thereby enhancing the bioavailablity of the active agent. Such delivery can offer several advantages over other modes of drug delivery, including, but not limited to, increasing the onset of action, lowering the required dosage, enhancing the efficacy, and improving the safety profile of the active agent. For example, tablet dosage forms enter the bloodstream through the gastrointestinal tract, which subjects the drug to degradation from stomach acid, bile, digestive enzymes, and other first pass metabolism effects. As a result, tablet formulations often require higher doses and generally have a delayed onset of action. Nasal administration of a drug also can facilitate compliance, especially for pediatric patients, geriatric patients, patients suffering from a neurodegenerative disease, or other patients for which swallowing is difficult, e.g., patients suffering from nausea, such as patients undergoing chemotherapy, or patients with a swallowing disorder.

Intranasal (“i.n.” or “IN”) delivery of an agent to a subject can facilitate delivery of the agent to the brain and/or peripheral nervous system. Such administration is non-invasive and offers several advantages including avoidance of hepatic first pass clearance, rapid onset of action, frequent self-administration and easy dose adjustments. Small molecules have an added advantage of being absorbed paracellularly through the nasal epithelium after which, these molecules can then directly enter the CNS through the olfactory or the trigeminal nerve associated pathway and can be directly transported to the brain upon intranasal administration.

For intranasal delivery, in addition to the active ingredients, pharmaceutical compositions may contain suitable pharmaceutically acceptable carriers comprising excipients and auxiliaries which facilitate processing of the active compounds into preparations which can be used pharmaceutically. The agents of the disclosure may be formulated by methods known to those of skill in the art, and may include, for example, but not limited to, examples of solubilizing, diluting, or dispersing substances, such as saline, preservatives, such as benzyl alcohol, absorption promoters, and fluorocarbons. Optimized formulations for intranasal delivery may include addition of permeability enhancers (mucoadhesives, nanoparticles, and the like) as well as combined use with an intranasal drug delivery device (for example, one that provides controlled particle dispersion with particles aerosolized to target the upper nasal cavity).

In particular, polymer-based nanoparticles, including chitosan, maltodextrin, polyethylene glycol (PEG), polylactic acid (PLA), polylactic-co-gly colic acid (PLGA), and PAMAM dendrimer; gels, including poloxamer; and lipid-based formulations, including glycerol monocaprate (Capmuil™), mixtures of mono-, di-, and triglycerides and mono- and di-fatty esters of PEG (Labrafil™), palmitate, glycerol monostearate, and phospholipids can be used to administer the presently disclosed GCP-II inhibitors intranasally.

The presently disclosed GCP-II inhibitors also can be administered intranasally via mucoadhesive agents. Mucoadhesion is commonly defined as the adhesion between two materials, at least one of which is a mucosal surface. More particularly, mucoadhesion is the interaction between a mucin surface and a synthetic or natural polymer. Mucoadhesive dosage forms can be designed to enable prolonged retention at the site of application, providing a controlled rate of drug release for improved therapeutic outcome. Application of dosage forms to mucosal surfaces may be of benefit to drug molecules not amenable to the oral route, such as those that undergo acid degradation or extensive first-pass metabolism. Mucoadhesive materials suitable for use with nasal administration of the presently disclosed GCP-II inhibitors include, but are not limited to, soluble cellulose derivatives, such as hydroxypropyl methylcellulose (HPMC), hydroxypropyl cellulose (HPC), methylcellulose (MC), and carboxymethyl cellulose (CMC), and insoluble cellulose derivatives, such as ethylcellulose and microcrystalline cellulose (MCC), starch (e.g., Amioca®), polyacrylates, such as poly(acrylic acid) (e.g., Carbopol® 974P), functionalized mucoadhesive polymers, such as polycarbophil, hyaluronan, and amberlite resin, and chitosan (2-amino-2-deoxy-(1→4)-β-d-glucopyranan) formulations and derivatives thereof.

In some embodiments, the formulation also includes a permeability enhancer. As used herein, the term “permeability enhancer” refers to a substance that facilitates the delivery of a drug across mucosal tissue. The term encompasses chemical enhancers that, when applied to the mucosal tissue, render the tissue more permeable to the drug. Permeability enhancers include, but are not limited to, dimethyl sulfoxide (DMSO), hydrogen peroxide (H₂O₂), propylene glycol, oleic acid, cetyl alcohol, benzalkoniurm chloride, sodium lauryl sulphate, isopropyl myristate, Tween 80, dimethyl formamide, dimethyl acetamide, sodium lauroylsarcosinate, sorbitan monolaurate, methylsulfonylmethane, Azone, terpenes, phosphatidylcholine dependent phospholipase C, triacyl glycerol hydrolase, acid phosphatase, phospholipase A2, concentrated saline solutions (e.g., PBS and NaCl), polysorbate 80, polysorbate 20, sodium dodecanoate (C12), sodium caprate (CIO) and/or sodium palmitate (CI 6), tert-butyl cyclohexanol (TBCH), and alpha-terpinol.

In some embodiments, the intranasal administration is accomplished via a ViaNase™ device (Kurve Technology, Inc.).

Pharmaceutical compositions suitable for use in the present disclosure include compositions wherein the active ingredients are contained in an effective amount to achieve its intended purpose. Determination of the effective amounts is well within the capability of those skilled in the art, especially in light of the detailed disclosure provided herein. Generally, the compounds according to the disclosure are effective over a wide dosage range. For example, in the treatment of adult humans, dosages from 0.01 to 1000 mg, from 0.5 to 100 mg, from 1 to 50 mg per day, and from 5 to 40 mg per day are examples of dosages that may be used. A non-limiting dosage is 10 to 30 mg per day. The exact dosage will depend upon the route of administration, the form in which the compound is administered, the subject to be treated, the body weight of the subject to be treated, the bioavailability of the compound(s), the adsorption, distribution, metabolism, and excretion (ADME) toxicity of the compound(s), and the preference and experience of the attending physician.

In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions.

Pharmaceutical preparations for oral use can be obtained by combining the active compounds with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl-cellulose, sodium carboxymethyl-cellulose (CMC), and/or polyvinylpyrrolidone (PVP: povidone). If desired, disintegrating agents may be added, such as the cross-linked polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol (PEG), and/or titanium dioxide, lacquer solutions, and suitable organic solvents or solvent mixtures. Dye-stuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin, and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols (PEGs). In addition, stabilizers may be added.

### G. Definitions

Although specific terms are employed herein, they are used in a generic and descriptive sense only and not for purposes of limitation. Unless otherwise defined, all technical and scientific terms used herein have the same meaning as commonly understood by one of ordinary skill in the art to which this presently described subject matter belongs.

While the following terms in relation to compounds of formulae (I-III) are believed to be well understood by one of ordinary skill in the art, the following definitions are set forth to facilitate explanation of the presently disclosed subject matter. These definitions are intended to supplement and illustrate, not preclude, the definitions that would be apparent to one of ordinary skill in the art upon review of the present disclosure.

The terms substituted, whether preceded by the term “optionally” or not, and substituent, as used herein, refer to the ability, as appreciated by one skilled in this art, to change one functional group for another functional group on a molecule, provided that the valency of all atoms is maintained. When more than one position in any given structure may be substituted with more than one substituent selected from a specified group, the substituent may be either the same or different at every position. The substituents also may be further substituted (e.g., an aryl group substituent may have another substituent off it, such as another aryl group, which is further substituted at one or more positions).

Where substituent groups or linking groups are specified by their conventional chemical formulae, written from left to right, they equally encompass the chemically identical substituents that would result from writing the structure from right to left, e.g., —CH₂O— is equivalent to —OCH₂—; —C(═O)O— is equivalent to —OC(═O)—; —OC(═O)NR— is equivalent to —NRC(═O)O—, and the like.

When the term “independently selected” is used, the substituents being referred to (e.g., R groups, such as groups R₁, R₂, and the like, or variables, such as “m” and “n”), can be identical or different. For example, both R₁ and R₂ can be substituted alkyls, or R₁ can be hydrogen and R₂ can be a substituted alkyl, and the like.

The terms “a,” “an,” or “a(n),” when used in reference to a group of substituents herein, mean at least one. For example, where a compound is substituted with “an” alkyl or aryl, the compound is optionally substituted with at least one alkyl and/or at least one aryl. Moreover, where a moiety is substituted with an R substituent, the group may be referred to as “R-substituted.” Where a moiety is R-substituted, the moiety is substituted with at least one R substituent and each R substituent is optionally different.

A named “R” or group will generally have the structure that is recognized in the art as corresponding to a group having that name, unless specified otherwise herein. For the purposes of illustration, certain representative “R” groups as set forth above are defined below.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

Unless otherwise explicitly defined, a “substituent group,” as used herein, includes a functional group selected from one or more of the following moieties, which are defined herein:

The term hydrocarbon, as used herein, refers to any chemical group comprising hydrogen and carbon. The hydrocarbon may be substituted or unsubstituted. As would be known to one skilled in this art, all valencies must be satisfied in making any substitutions. The hydrocarbon may be unsaturated, saturated, branched, unbranched, cyclic, polycyclic, or heterocyclic. Illustrative hydrocarbons are further defined herein below and include, for example, methyl, ethyl, n-propyl, isopropyl, cyclopropyl, allyl, vinyl, n-butyl, tert-butyl, ethynyl, cyclohexyl, and the like.

The term “alkyl,” by itself or as part of another substituent, means, unless otherwise stated, a straight (i.e., unbranched) or branched chain, acyclic or cyclic hydrocarbon group, or combination thereof, which may be fully saturated, mono- or polyunsaturated and can include di- and multivalent groups, having the number of carbon atoms designated (i.e., C₁₋₁₀ means one to ten carbons, including 1, 2, 3, 4, 5, 6, 7, 8, 9, and 10 carbons). In particular embodiments, the term “alkyl” refers to C₁₋₂₀ inclusive, including 1, 2, 3, 4, 5, 6, 7, 8, 9, 10, 11, 12, 13, 14, 15, 16, 17, 18, 19, and 20 carbons, linear (i.e., “straight-chain”), branched, or cyclic, saturated or at least partially and in some cases fully unsaturated (i.e., alkenyl and alkynyl) hydrocarbon radicals derived from a hydrocarbon moiety containing between one and twenty carbon atoms by removal of a single hydrogen atom.

Representative saturated hydrocarbon groups include, but are not limited to, methyl, ethyl, n-propyl, isopropyl, n-butyl, isobutyl, sec-butyl, tert-butyl, n-pentyl, sec-pentyl, isopentyl, neopentyl, n-hexyl, sec-hexyl, n-heptyl, n-octyl, n-decyl, n-undecyl, dodecyl, cyclohexyl, (cyclohexyl)methyl, cyclopropylmethyl, and homologs and isomers thereof.

“Branched” refers to an alkyl group in which a lower alkyl group, such as methyl, ethyl or propyl, is attached to a linear alkyl chain. “Lower alkyl” refers to an alkyl group having 1 to about 8 carbon atoms (i.e., a C₁₋₈ alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms. In certain embodiments, “alkyl” refers, in particular, to C₁₋₈ straight-chain alkyls. In other embodiments, “alkyl” refers, in particular, to C₁₋₈ branched-chain alkyls.

Alkyl groups can optionally be substituted (a “substituted alkyl”) with one or more alkyl group substituents, which can be the same or different. The term “alkyl group substituent” includes but is not limited to alkyl, substituted alkyl, halo, arylamino, acyl, hydroxyl, aryloxyl, alkoxyl, alkylthio, arylthio, aralkyloxyl, aralkylthio, carboxyl, alkoxycarbonyl, oxo, and cycloalkyl. There can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, as used herein, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, amino, alkylamino, dialkylamino, sulfate, cyano, and mercapto.

The term “heteroalkyl,” by itself or in combination with another term, means, unless otherwise stated, a stable straight or branched chain having from 1 to 20 carbon atoms or heteroatoms or a cyclic hydrocarbon group having from 3 to 10 carbon atoms or heteroatoms, or combinations thereof, consisting of at least one carbon atom and at least one heteroatom selected from the group consisting of O, N, P, Si and S, and wherein the nitrogen, phosphorus, and sulfur atoms may optionally be oxidized and the nitrogen heteroatom may optionally be quaternized. The heteroatom(s) O, N, P and S and Si may be placed at any interior position of the heteroalkyl group or at the position at which alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, O—CH₃, —O—CH₂—CH₃, and —CN. Up to two or three heteroatoms may be consecutive, such as, for example, —CH₂—NH—OCH₃ and —CH₂—O—Si(CH₃)₃.

As described above, heteroalkyl groups, as used herein, include those groups that are attached to the remainder of the molecule through a heteroatom, such as —C(O)NR′, —NR′R″, —OR′, —SR, —S(O)R, and/or —S(O₂)R′. Where “heteroalkyl” is recited, followed by recitations of specific heteroalkyl groups, such as —NR′R or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

“Cyclic” and “cycloalkyl” refer to a non-aromatic mono- or multicyclic ring system of about 3 to about 10 carbon atoms, e.g., 3, 4, 5, 6, 7, 8, 9, or 10 carbon atoms. The cycloalkyl group can be optionally partially unsaturated. The cycloalkyl group also can be optionally substituted with an alkyl group substituent as defined herein, oxo, and/or alkylene. There can be optionally inserted along the cyclic alkyl chain one or more oxygen, sulfur or substituted or unsubstituted nitrogen atoms, wherein the nitrogen substituent is hydrogen, unsubstituted alkyl, substituted alkyl, aryl, or substituted aryl, thus providing a heterocyclic group. Representative monocyclic cycloalkyl rings include cyclopentyl, cyclohexyl, and cycloheptyl. Multicyclic cycloalkyl rings include adamantyl, octahydronaphthyl, decalin, camphor, camphane, and noradamantyl, and fused ring systems, such as dihydro- and tetrahydronaphthalene, and the like.

The term “cycloalkylalkyl,” as used herein, refers to a cycloalkyl group as defined hereinabove, which is attached to the parent molecular moiety through an alkylene moiety, also as defined above, e.g., a C₁₋₂₀ alkylene moiety. Examples of cycloalkylalkyl groups include cyclopropylmethyl and cyclopentylethyl.

The terms “cycloheteroalkyl” or “heterocycloalkyl” refer to a non-aromatic ring system, unsaturated or partially unsaturated ring system, such as a 3- to 10-member substituted or unsubstituted cycloalkyl ring system, including one or more heteroatoms, which can be the same or different, and are selected from the group consisting of nitrogen (N), oxygen (O), sulfur (S), phosphorus (P), and silicon (Si), and optionally can include one or more double bonds.

The cycloheteroalkyl ring can be optionally fused to or otherwise attached to other cycloheteroalkyl rings and/or non-aromatic hydrocarbon rings. Heterocyclic rings include those having from one to three heteroatoms independently selected from oxygen, sulfur, and nitrogen, in which the nitrogen and sulfur heteroatoms may optionally be oxidized and the nitrogen heteroatom may optionally be quaternized. In certain embodiments, the term heterocylic refers to a non-aromatic 5-, 6-, or 7-membered ring or a polycyclic group wherein at least one ring atom is a heteroatom selected from O, S, and N (wherein the nitrogen and sulfur heteroatoms may be optionally oxidized), including, but not limited to, a bi- or tri-cyclic group, comprising fused six-membered rings having between one and three heteroatoms independently selected from the oxygen, sulfur, and nitrogen, wherein (i) each 5-membered ring has 0 to 2 double bonds, each 6-membered ring has 0 to 2 double bonds, and each 7-membered ring has 0 to 3 double bonds, (ii) the nitrogen and sulfur heteroatoms may be optionally oxidized, (iii) the nitrogen heteroatom may optionally be quaternized, and (iv) any of the above heterocyclic rings may be fused to an aryl or heteroaryl ring. Representative cycloheteroalkyl ring systems include, but are not limited to pyrrolidinyl, pyrrolinyl, imidazolidinyl, imidazolinyl, pyrazolidinyl, pyrazolinyl, piperidinyl, piperazinyl, indolinyl, quinuclidinyl, morpholinyl, thiomorpholinyl, thiadiazinanyl, tetrahydrofuranyl, and the like.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. The terms “cycloalkylene” and “heterocycloalkylene” refer to the divalent derivatives of cycloalkyl and heterocycloalkyl, respectively.

As used herein the terms “bicycloalkyl” and “bicycloheteroalkyl” refer to two cycloalkyl or cycloheteroalkyl groups that are bound to one another. Non-limiting examples include bicyclohexane and bipiperidine.

An unsaturated hydrocarbon has one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. Alkyl groups which are limited to hydrocarbon groups are termed “homoalkyl.”

More particularly, the term “alkenyl” as used herein refers to a monovalent group derived from a C₂₋₂₀ inclusive straight or branched hydrocarbon moiety having at least one carbon-carbon double bond by the removal of a single hydrogen molecule. Alkenyl groups include, for example, ethenyl (i.e., vinyl), propenyl, butenyl, 1-methyl-2-buten-1-yl, pentenyl, hexenyl, octenyl, allenyl, and butadienyl.

The term “cycloalkenyl” as used herein refers to a cyclic hydrocarbon containing at least one carbon-carbon double bond. Examples of cycloalkenyl groups include cyclopropenyl, cyclobutenyl, cyclopentenyl, cyclopentadiene, cyclohexenyl, 1,3-cyclohexadiene, cycloheptenyl, cycloheptatrienyl, and cyclooctenyl.

The term “alkynyl” as used herein refers to a monovalent group derived from a straight or branched C₂₋₂₀ hydrocarbon of a designed number of carbon atoms containing at least one carbon-carbon triple bond. Examples of “alkynyl” include ethynyl, 2-propynyl (propargyl), 1-propynyl, pentynyl, hexynyl, and heptynyl groups, and the like.

The term “alkylene” by itself or a part of another substituent refers to a straight or branched bivalent aliphatic hydrocarbon group derived from an alkyl group having from 1 to about 20 carbon atoms, e.g., 1, 2, 3, 4, 5, 6, 7, 8, 9, 10, 11, 12, 13, 14, 15, 16, 17, 18, 19, or 20 carbon atoms. The alkylene group can be straight, branched or cyclic. The alkylene group also can be optionally unsaturated and/or substituted with one or more “alkyl group substituents.” There can be optionally inserted along the alkylene group one or more oxygen, sulfur or substituted or unsubstituted nitrogen atoms (also referred to herein as “alkylaminoalkyl”), wherein the nitrogen substituent is alkyl as previously described. Exemplary alkylene groups include methylene (—CH₂—); ethylene (—CH₂—CH₂—); propylene (—(CH₂)₃—); cyclohexylene (—C₆H₁₀—); —CH═CH—CH═CH—; —CH═CH—CH₂—; —CH₂CH₂CH₂CH₂—, —CH₂CH═CHCH₂—, —CH₂CsCCH₂—, —CH₂CH₂CH(CH₂CH₂CH₃)CH₂—, —(CH₂)_{q}—N(R)—(CH₂)ᵣ—, wherein each of q and r is independently an integer from 0 to about 20, e.g., 0, 1, 2, 3, 4, 5, 6, 7, 8, 9, 10, 11, 12, 13, 14, 15, 16, 17, 18, 19, or 20, and R is hydrogen or lower alkyl; methylenedioxyl (—O—CH₂—O—); and ethylenedioxyl (—O—(CH₂)₂—O—). An alkylene group can have about 2 to about 3 carbon atoms and can further have 6-20 carbons. Typically, an alkyl (or alkylene) group will have from 1 to 24 carbon atoms, with those groups having 10 or fewer carbon atoms being some embodiments of the present disclosure. A “lower alkyl” or “lower alkylene” is a shorter chain alkyl or alkylene group, generally having eight or fewer carbon atoms.

The term “heteroalkylene” by itself or as part of another substituent means a divalent group derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms also can occupy either or both of the chain termini (e.g., alkyleneoxo, alkylenedioxo, alkyleneamino, alkylenediamino, and the like). Still further, for alkylene and heteroalkylene linking groups, no orientation of the linking group is implied by the direction in which the formula of the linking group is written. For example, the formula —C(O)OR′— represents both —C(O)OR′— and —R′OC(O)—.

The term “aryl” means, unless otherwise stated, an aromatic hydrocarbon substituent that can be a single ring or multiple rings (such as from 1 to 3 rings), which are fused together or linked covalently. The term “heteroaryl” refers to aryl groups (or rings) that contain from one to four heteroatoms (in each separate ring in the case of multiple rings) selected from N, O, and S, wherein the nitrogen and sulfur atoms are optionally oxidized, and the nitrogen atom(s) are optionally quaternized. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of above noted aryl and heteroaryl ring systems are selected from the group of acceptable substituents described below. The terms “arylene” and “heteroarylene” refer to the divalent forms of aryl and heteroaryl, respectively.

For brevity, the term “aryl” when used in combination with other terms (e.g., aryloxy, arylthioxy, arylalkyl) includes both aryl and heteroaryl rings as defined above. Thus, the terms “arylalkyl” and “heteroarylalkyl” are meant to include those groups in which an aryl or heteroaryl group is attached to an alkyl group (e.g., benzyl, phenethyl, pyridylmethyl, furylmethyl, and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like). However, the term “haloaryl,” as used herein is meant to cover only aryls substituted with one or more halogens.

Where a heteroalkyl, heterocycloalkyl, or heteroaryl includes a specific number of members (e.g. “3 to 7 membered”), the term “member” refers to a carbon or heteroatom.

Further, a structure represented generally by the formula:

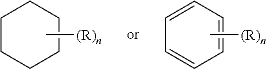

as used herein refers to a ring structure, for example, but not limited to a 3-carbon, a 4-carbon, a 5-carbon, a 6-carbon, a 7-carbon, and the like, aliphatic and/or aromatic cyclic compound, including a saturated ring structure, a partially saturated ring structure, and an unsaturated ring structure, comprising a substituent R group, wherein the R group can be present or absent, and when present, one or more R groups can each be substituted on one or more available carbon atoms of the ring structure. The presence or absence of the R group and number of R groups is determined by the value of the variable “n,” which is an integer generally having a value ranging from 0 to the number of carbon atoms on the ring available for substitution. Each R group, if more than one, is substituted on an available carbon of the ring structure rather than on another R group. For example, the structure above where n is 0 to 2 would comprise compound groups including, but not limited to:

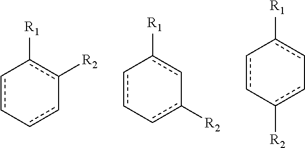

and the like.

A dashed line representing a bond in a cyclic ring structure indicates that the bond can be either present or absent in the ring. That is, a dashed line representing a bond in a cyclic ring structure indicates that the ring structure is selected from the group consisting of a saturated ring structure, a partially saturated ring structure, and an unsaturated ring structure.

The symbol ( [symbol] ) denotes the point of attachment of a moiety to the remainder of the molecule.

When a named atom of an aromatic ring or a heterocyclic aromatic ring is defined as being “absent,” the named atom is replaced by a direct bond.

Each of above terms (e.g., “alkyl,” “heteroalkyl,” “cycloalkyl, and “heterocycloalkyl”, “aryl,” “heteroaryl,” “phosphonate,” and “sulfonate” as well as their divalent derivatives) are meant to include both substituted and unsubstituted forms of the indicated group. Optional substituents for each type of group are provided below.

Substituents for alkyl, heteroalkyl, cycloalkyl, heterocycloalkyl monovalent and divalent derivative groups (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to: —OR′, =O, =NR′, =N—OR′, —NR′R″, —SR′, -halogen, —SiR′R″R′″, —OC(O)R′, —C(O)R′, —CO₂R′, —C(O)NR′R″, —OC(O)NR′R″, —NR″C(O)R′, —NR′—C(O)NR″R′″, —NR″C(O)OR′, —NR—C(NR′R″)=NR′″, —S(O)R′, —S(O)₂R′, —S(O)₂NR′R″, —NRSO₂R′, —CN, CF₃, fluorinated C₁₋₄ alkyl, and —NO₂ in a number ranging from zero to (2m′+1), where m′ is the total number of carbon atoms in such groups. R′, R″, R′″ and R″″ each may independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy or thioalkoxy groups, or arylalkyl groups. As used herein, an “alkoxy” group is an alkyl attached to the remainder of the molecule through a divalent oxygen. When a compound of the disclosure includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 4-, 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for alkyl groups above, exemplary substituents for aryl and heteroaryl groups (as well as their divalent derivatives) are varied and are selected from, for example: halogen, —OR′, —NR′R″, —SR′, —SiR′R″R′″, —OC(O)R′, —C(O)R′, —CO₂R′, —C(O)NR′R″, —OC(O)NR′R″, —NR″C(O)R′, —NR′—C(O)NR″R′″, —NR″C(O)OR′, —NR—C(NR′R″R′″)=NR″″, —NR—C(NR′R″)=NR′″ —S(O)R′, —S(O)₂R′, —S(O)₂NR′R″, —NRSO₂R′, —CN and —NO₂, —R′, —N₃, —CH(Ph)₂, fluoro(C₁₋₄)alkoxo, and fluoro(C₁₋₄)alkyl, in a number ranging from zero to the total number of open valences on aromatic ring system; and where R′, R″, R′″ and R″″ may be independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. When a compound of the disclosure includes more than one R group, for example, each of the R groups is independently selected as are each R′, R″, R′″ and R″″ groups when more than one of these groups is present.

Two of the substituents on adjacent atoms of aryl or heteroaryl ring may optionally form a ring of the formula -T-C(O)—(CRR′)_{q}—U—, wherein T and U are independently —NR—, —O—, —CRR′— or a single bond, and q is an integer of from 0 to 3. Alternatively, two of the substituents on adjacent atoms of aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4.

One of the single bonds of the new ring so formed may optionally be replaced with a double bond. Alternatively, two of the substituents on adjacent atoms of aryl or heteroaryl ring may optionally be replaced with a substituent of the formula —(CRR′)ₛ—X′—(C″R′″)_{d}—, where s and d are independently integers of from 0 to 3, and X′ is —O—, —NR′—, —S—, —S(O)—, —S(O)₂—, or —S(O)₂NR′—. The substituents R, R′, R″ and R′″ may be independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

As used herein, the term “acyl” refers to an organic acid group wherein the —OH of the carboxyl group has been replaced with another substituent and has the general formula RC(═O)—, wherein R is an alkyl, alkenyl, alkynyl, aryl, carbocylic, heterocyclic, or aromatic heterocyclic group as defined herein). As such, the term “acyl” specifically includes arylacyl groups, such as a 2-(furan-2-yl)acetyl)- and a 2-phenylacetyl group. Specific examples of acyl groups include acetyl and benzoyl. Acyl groups also are intended to include amides, —RC(═O)NR′, esters, —RC(═O)OR′, ketones, —RC(═O)R′, and aldehydes, —RC(═O)H.

The terms “alkoxyl” or “alkoxy” are used interchangeably herein and refer to a saturated (i.e., alkyl-O—) or unsaturated (i.e., alkenyl-O— and alkynyl-O—) group attached to the parent molecular moiety through an oxygen atom, wherein the terms “alkyl,” “alkenyl,” and “alkynyl” are as previously described and can include C₁₋₂₀ inclusive, linear, branched, or cyclic, saturated or unsaturated oxo-hydrocarbon chains, including, for example, methoxyl, ethoxyl, propoxyl, isopropoxyl, n-butoxyl, sec-butoxyl, tert-butoxyl, and n-pentoxyl, neopentoxyl, n-hexoxyl, and the like.

The term “alkoxyalkyl” as used herein refers to an alkyl-O-alkyl ether, for example, a methoxyethyl or an ethoxymethyl group.

“Aryloxyl” refers to an aryl-O— group wherein the aryl group is as previously described, including a substituted aryl. The term “aryloxyl” as used herein can refer to phenyloxyl or hexyloxyl, and alkyl, substituted alkyl, halo, or alkoxyl substituted phenyloxyl or hexyloxyl.

“Aralkyl” refers to an aryl-alkyl-group wherein aryl and alkyl are as previously described, and included substituted aryl and substituted alkyl. Exemplary aralkyl groups include benzyl, phenylethyl, and naphthylmethyl.

“Aralkyloxyl” refers to an aralkyl-O— group wherein the aralkyl group is as previously described. An exemplary aralkyloxyl group is benzyloxyl, i.e., C₆H₅—CH₂—O—. An aralkyloxyl group can optionally be substituted.

“Alkoxycarbonyl” refers to an alkyl-O—C(═O)— group. Exemplary alkoxycarbonyl groups include methoxycarbonyl, ethoxycarbonyl, butyloxycarbonyl, and tert-butyloxycarbonyl.

“Aryloxycarbonyl” refers to an aryl-O—C(═O)— group. Exemplary aryloxycarbonyl groups include phenoxy- and naphthoxy-carbonyl.

“Aralkoxycarbonyl” refers to an aralkyl-O—C(═O)— group. An exemplary aralkoxycarbonyl group is benzyloxycarbonyl.

“Carbamoyl” refers to an amide group of the formula —C(═O)NH₂.

“Alkylcarbamoyl” refers to a R′RN—C(═O)— group wherein one of R and R′ is hydrogen and the other of R and R′ is alkyl and/or substituted alkyl as previously described.

“Dialkylcarbamoyl” refers to a R′RN—C(═O)— group wherein each of R and R′ is independently alkyl and/or substituted alkyl as previously described.

The term carbonyldioxyl, as used herein, refers to a carbonate group of the formula —O—C(═O)—OR.

“Acyloxyl” refers to an acyl-O— group wherein acyl is as previously described.

The term “amino” refers to the —NH₂ group and also refers to a nitrogen containing group as is known in the art derived from ammonia by the replacement of one or more hydrogen radicals by organic radicals. For example, the terms “acylamino” and “alkylamino” refer to specific N-substituted organic radicals with acyl and alkyl substituent groups respectively.

An “aminoalkyl” as used herein refers to an amino group covalently bound to an alkylene linker. More particularly, the terms alkylamino, dialkylamino, and trialkylamino as used herein refer to one, two, or three, respectively, alkyl groups, as previously defined, attached to the parent molecular moiety through a nitrogen atom. The term alkylamino refers to a group having the structure —NHR′ wherein R′ is an alkyl group, as previously defined; whereas the term dialkylamino refers to a group having the structure —NR′R″, wherein R′ and R″ are each independently selected from the group consisting of alkyl groups. The term trialkylamino refers to a group having the structure —NR′R″R′″, wherein R′, R″, and R′ are each independently selected from the group consisting of alkyl groups. Additionally, R′, R″, and/or R′″ taken together may optionally be —(CH₂)ₖ— where k is an integer from 2 to 6. Examples include, but are not limited to, methylamino, dimethylamino, ethylamino, diethylamino, diethylaminocarbonyl, methylethylamino, isopropylamino, piperidino, trimethylamino, and propylamino.

The amino group is —NR′R″, wherein R′ and R″ are typically selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

The terms alkylthioether and thioalkoxyl refer to a saturated (i.e., alkyl-S—) or unsaturated (i.e., alkenyl-S— and alkynyl-S—) group attached to the parent molecular moiety through a sulfur atom. Examples of thioalkoxyl moieties include, but are not limited to, methylthio, ethylthio, propylthio, isopropylthio, n-butylthio, and the like.

“Acylamino” refers to an acyl-NH— group wherein acyl is as previously described. “Aroylamino” refers to an aroyl-NH— group wherein aroyl is as previously described.

The term “carbonyl” refers to the —C(═O)— group, and can include an aldehyde group represented by the general formula R—C(═O)H.

The term “carboxyl” refers to the —COOH group. Such groups also are referred to herein as a “carboxylic acid” moiety.

The term “cyano” refers to the —C≡N group.

The terms “halo,” “halide,” or “halogen” as used herein refer to fluoro, chloro, bromo, and iodo groups. Additionally, terms such as “haloalkyl,” are meant to include monohaloalkyl and polyhaloalkyl. For example, the term “halo(C₁₋₄)alkyl” is mean to include, but not be limited to, trifluoromethyl, 2,2,2-trifluoroethyl, 4-chlorobutyl, 3-bromopropyl, and the like.

The term “hydroxyl” refers to the —OH group.

The term “hydroxyalkyl” refers to an alkyl group substituted with an —OH group.

The term “mercapto” refers to the —SH group.

The term “oxo” as used herein means an oxygen atom that is double bonded to a carbon atom or to another element.

The term “nitro” refers to the —NO₂ group.

The term “thio” refers to a compound described previously herein wherein a carbon or oxygen atom is replaced by a sulfur atom.

The term “sulfate” refers to the —SO₄ group.

The term thiohydroxyl or thiol, as used herein, refers to a group of the formula —SH.

More particularly, the term “sulfide” refers to compound having a group of the formula —SR.

The term “sulfone” refers to compound having a sulfonyl group —S(O₂)R.

The term “sulfoxide” refers to a compound having a sulfinyl group —S(O)R

The term ureido refers to a urea group of the formula —NH—CO—NH₂.

Throughout the specification and claims, a given chemical formula or name shall encompass all tautomers, congeners, and optical- and stereoisomers, as well as racemic mixtures where such isomers and mixtures exist.

Certain compounds of the present disclosure may possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as D- or L- for amino acids, and individual isomers are encompassed within the scope of the present disclosure. The compounds of the present disclosure do not include those which are known in art to be too unstable to synthesize and/or isolate. The present disclosure is meant to include compounds in racemic, scalemic, and optically pure forms. Optically active (R)- and (S)-, or D- and L-isomers may be prepared using chiral synthons or chiral reagents, or resolved using conventional techniques. When the compounds described herein contain olefenic bonds or other centers of geometric asymmetry, and unless specified otherwise, it is intended that the compounds include both E and Z geometric isomers.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

It will be apparent to one skilled in the art that certain compounds of this disclosure may exist in tautomeric forms, all such tautomeric forms of the compounds being within the scope of the disclosure. The term “tautomer,” as used herein, refers to one of two or more structural isomers which exist in equilibrium and which are readily converted from one isomeric form to another.

Unless otherwise stated, structures depicted herein are also meant to include compounds which differ only in the presence of one or more isotopically enriched atoms. For example, compounds having the present structures with the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present disclosure, whether radioactive or not, are encompassed within the scope of the present disclosure.

The compounds of the present disclosure may exist as salts. The present disclosure includes such salts. Examples of applicable salt forms include hydrochlorides, hydrobromides, sulfates, methanesulfonates, nitrates, maleates, acetates, citrates, fumarates, tartrates (e.g. (+)-tartrates, (−)-tartrates or mixtures thereof including racemic mixtures, succinates, benzoates and salts with amino acids such as glutamic acid. These salts may be prepared by methods known to those skilled in art. Also included are base addition salts such as sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent or by ion exchange. Examples of acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like. Certain specific compounds of the present disclosure contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

In addition to salt forms, the present disclosure provides compounds, which are in a prodrug form. Prodrugs of the compounds described herein are those compounds that readily undergo chemical changes under physiological conditions to provide the compounds of the present disclosure. Additionally, prodrugs can be converted to the compounds of the present disclosure by chemical or biochemical methods in an ex vivo environment. For example, prodrugs can be slowly converted to the compounds of the present disclosure when placed in a transdermal patch reservoir with a suitable enzyme or chemical reagent.

The term “protecting group” refers to chemical moieties that block some or all reactive moieties of a compound and prevent such moieties from participating in chemical reactions until the protective group is removed, for example, those moieties listed and described in T. W. Greene, P. G. M. Wuts, Protective Groups in Organic Synthesis, 3rd ed. John Wiley & Sons (1999). It may be advantageous, where different protecting groups are employed, that each (different) protective group be removable by a different means. Protective groups that are cleaved under totally disparate reaction conditions allow differential removal of such protecting groups. For example, protective groups can be removed by acid, base, and hydrogenolysis. Groups such as trityl, dimethoxytrityl, acetal and tert-butyldimethylsilyl are acid labile and may be used to protect carboxy and hydroxy reactive moieties in the presence of amino groups protected with Cbz groups, which are removable by hydrogenolysis, and Fmoc groups, which are base labile. Carboxylic acid and hydroxy reactive moieties may be blocked with base labile groups such as, without limitation, methyl, ethyl, and acetyl in the presence of amines blocked with acid labile groups such as tert-butyl carbamate or with carbamates that are both acid and base stable but hydrolytically removable.

Carboxylic acid and hydroxy reactive moieties may also be blocked with hydrolytically removable protective groups such as the benzyl group, while amine groups capable of hydrogen bonding with acids may be blocked with base labile groups such as Fmoc. Carboxylic acid reactive moieties may be blocked with oxidatively-removable protective groups such as 2,4-dimethoxybenzyl, while co-existing amino groups may be blocked with fluoride labile silyl carbamates.

Allyl blocking groups are useful in the presence of acid- and base-protecting groups since the former are stable and can be subsequently removed by metal or pi-acid catalysts. For example, an allyl-blocked carboxylic acid can be deprotected with a palladium(O)—catalyzed reaction in the presence of acid labile t-butyl carbamate or base-labile acetate amine protecting groups. Yet another form of protecting group is a resin to which a compound or intermediate may be attached. As long as the residue is attached to the resin, that functional group is blocked and cannot react. Once released from the resin, the functional group is available to react.

Typical blocking/protecting groups include, but are not limited to the following moieties:

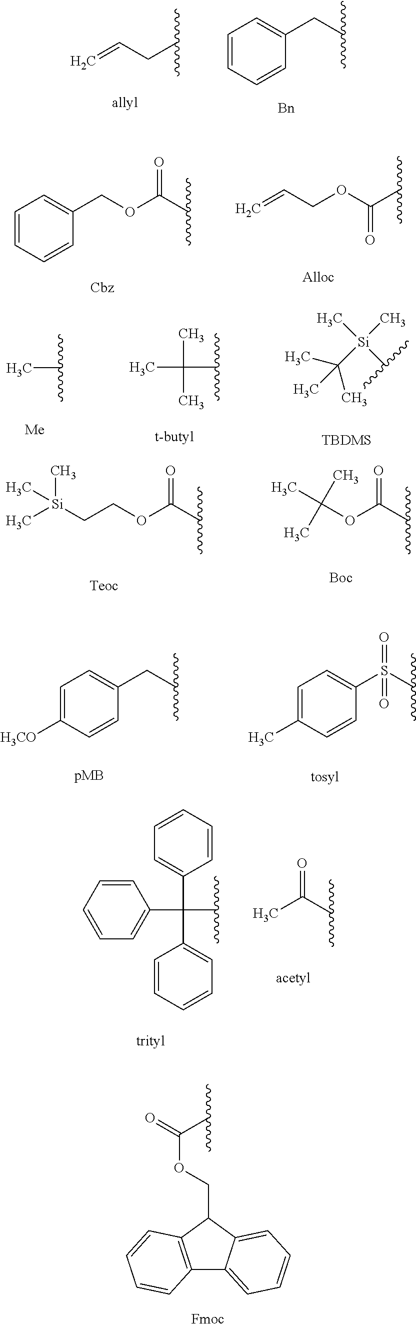

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

### GCPII Inhibition Prolongs Survival and Delays Muscle Mass, Frailty, and Function Loss in Aged C57BL6/J Mice

### 1.1 Overview

Sarcopenia is a common condition associated with aging where there is a substantial loss of muscle mass and function, resulting in a lowered quality of life, an increased fall risk, and is associated with poorer health outcomes. Although the precise mechanisms leading to sarcopenia is currently an area of active research, increased inflammation has been reported in both aging human and mouse muscle tissues and is thought to contribute toward sarcopenia.

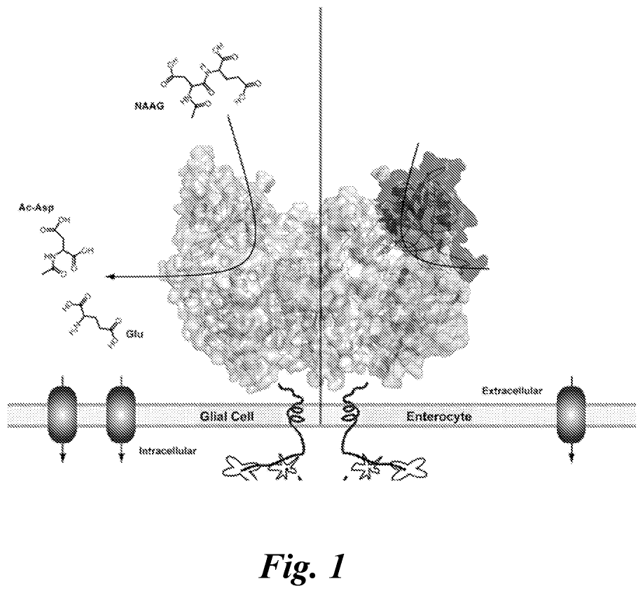

Glutamate carboxypeptidase II (GCPII), also referred to as FOLH1 and PSMA, is a neuropeptidase that catalyzes the conversion of N-acetyl-aspartyl-glutamate (NAAG) into N-acetyl-aspartate (NAA) and Glutamate (see FIG. 1). In the peripheral nervous system (PNS), GCPII is expressed in Schwann cells and activated macrophages and is involved in regulating the synaptic pruning of neuromuscular junctions (NMJs) during normal development.

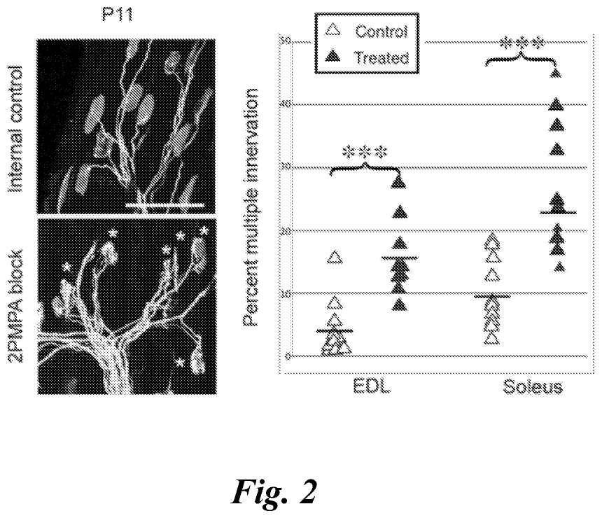

GCPII is highly expressed in brain and kidney and exhibits elevated expression on activated microglia/macrophage. Excess glutamate can be neurotoxic. Referring now to FIG. 2, excess glutamate causes axonal pruning in the developing PNS. Acetylcholine (Ach) is the canonical neurotransmitter at the NMJ. Glutamate is observed to play a role at the NMJ during normal muscle development. Inhibiting GCPII during development delayed synaptic pruning.

Increased GCPII expression was recently observed in the muscle of the SOD1G93A mouse model of amyotrophic lateral sclerosis (ALS) that was selectively associated with infiltrating activated macrophages. By inhibiting GCPII with dendrimer conjugated 2-(phosphonomethyl) pentanedioic acid (2-PMPA), a potent and selective inhibitor, a significant delay in muscle function loss and denervation was observed in the ALS mouse model.

ALS and aging share similarities including the loss of motor neurons and degeneration of the skeletal muscle resulting in reduced synaptic inputs and fine motor skills, muscle weakness or wasting, and neuro-inflammation. Due to these similarities, whether GCPII levels were altered in muscle of aged mice similarly to ALS mice was determined.

### 1.2 Results

### 1.2.1 GCPII Expression and Enzymatic Activity in Muscle Tissue of C57BL6/J Mice

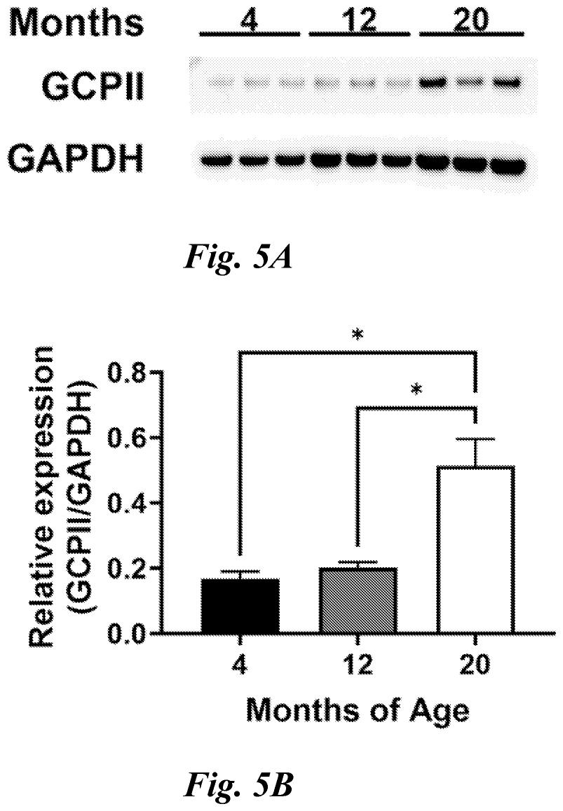

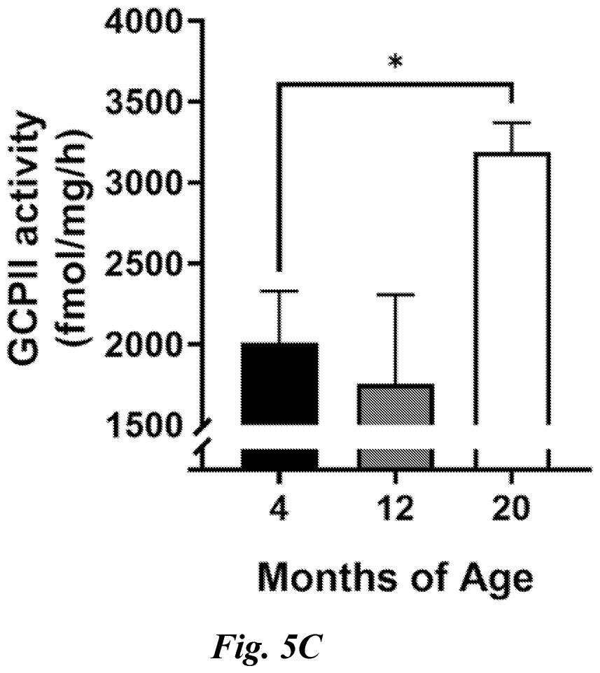

Gastrocnemius and soleus muscle tissue were collected from 4-, 12-, and 20-month-old C57BL/6 mice and GCPII protein expression and enzymatic activity levels were examined. Low expression of GCPII in 4-month-old and 12-month-old mice gastrocnemius muscle and increasing expression in 20-month-old mice was observed. Referring now to FIG. 5A, FIG. 5B, and FIG. 5C, GCPII protein and activity levels are increased in muscle from aged mice. FIG. 5A and FIG. 5B demonstrate that GCPII protein expression is increased in 20-month-old WT mice. FIG. 5C demonstrates that GCPII enzymatic activity is increased in 20-month-old WT mice. These data indicate that GCPII protein levels and enzymatic activity are elevated in the gastrocnemius muscle of aged, 20-month old WT C57BL6/J mice.

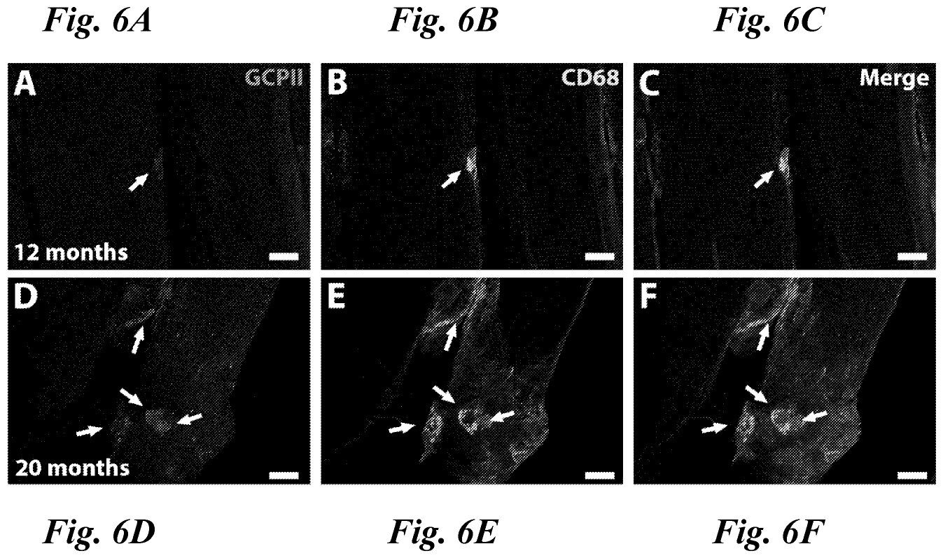

The elevated expression of GCPII in the mouse muscle is specifically associated with infiltrating, activated CD68+ macrophages. FIG. 6A, FIG. 6B, FIG. 6C, FIG. 6D, FIG. 6E, and FIG. 6F show that GCPII is expressed on activated macrophages in aged muscle.

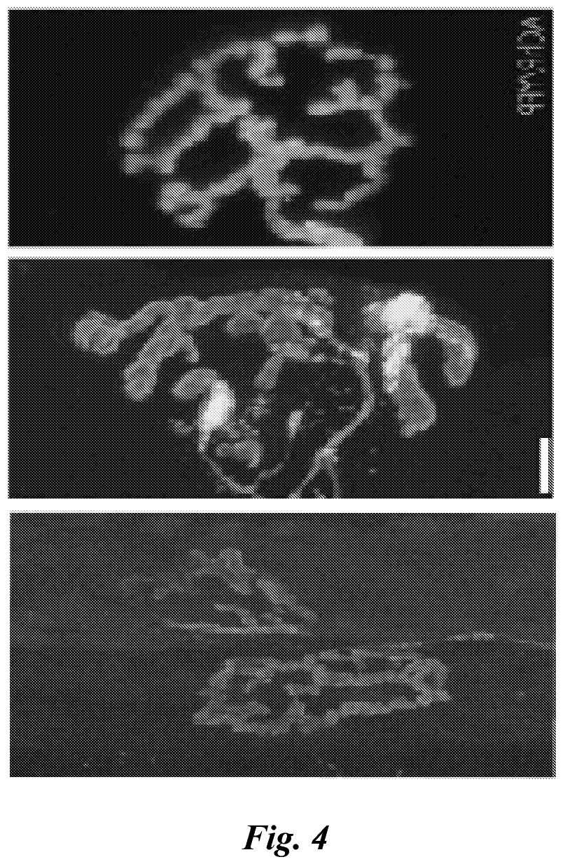

Like ALS, colocalization of GCPII staining on infiltrating activated macrophages in aged gastrocnemius muscle was observed. Further, a significant delay of muscle function loss and NMJ denervation in an ALS mouse model was observed using the potent GCPII inhibitor 2-(phosphonomethyl)-pentanedioic acid (2-PMPA) conjugated to a dendrimer molecule. As provided in FIG. 4, ALS and aging-related sarcopenia share pathological similarities, including progressive muscle weakness/wasting, Virenkumar et al., 2020; progressive muscle denervation and impaired regeneration, Virenkumar et al., 2020; motor neuron dysfunction/death, Virenkumar et al., 2020; elevated circulating plasma cytokines, Hu et al., 2017; Pan et al., 2021.

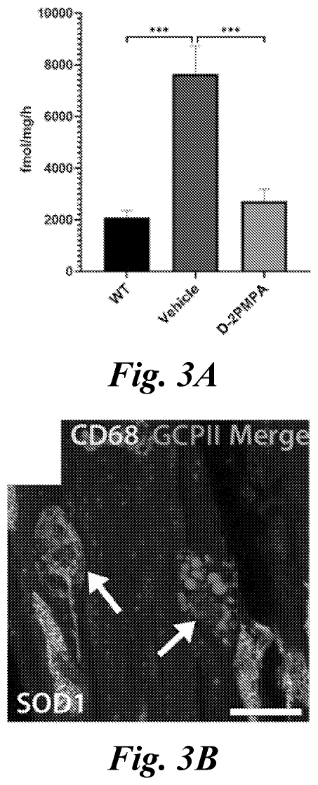

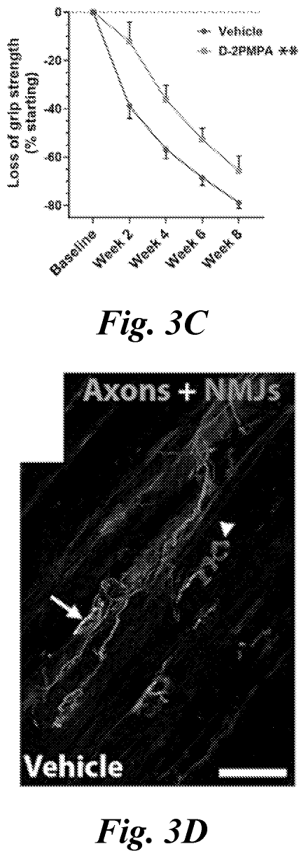

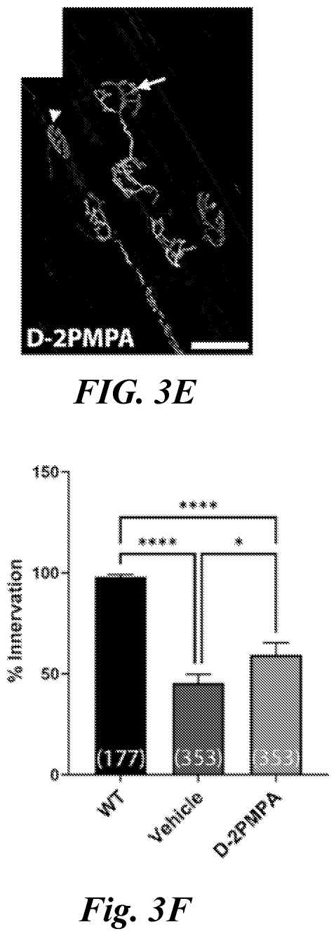

Referring now to FIG. 3A, FIG. 3B, FIG. 3C, FIG. 3D, FIG. 3E, and FIG. 3F, inhibiting elevated GCPII in an ALS mouse model delayed motor function loss and denervation. As provided in FIG. 3A, elevated GCPII activity was associated with activated macrophages in ALS mouse muscle. As provided in FIG. 3B, GCPII staining in the ALS mouse muscle colocalized with CD68+ macrophages. As provided in FIG. 3C, grip strength loss was delayed with GCPII inhibition. As provided in FIG. 3D, FIG. 3E, and FIG. 3F GCPII inhibition reduced muscle denervation.

To determine whether 2-PMPA treatment could delay muscle function loss in aged animals, 15-month old C57BL6/J mice were treated with 100 mg/kg of 2-PMPA IP daily for 5 months and monthly body weight, grip strength, compound muscle action potential (CMAP), open field behavior, end stage muscle weights and survival were monitored.

Treating 15-month old WT mice with 100 mg/kg of the prototypical GCPII inhibitor 2-PMPA 5 days per week for 5 months significantly improved grip strength, movement in open field, and electrophysiological recordings of the compound muscle action potential (CMAP) in the foot muscles. Compared to vehicle treated mice, a significant delay in body weight loss and grip strength loss were observed after 5 months on 2-PMPA therapy. For example, as early as 2 months on treatment, 2-PMPA treated animals had significantly greater calf muscle area compared to vehicle treated mice as measured by MRI. After 5 months of treatment, 2-PMPA treated animals were moving more in the open field apparatus. By 5 months, 2-PMPA treated animals had significantly increased gastrocnemius muscle weights and exhibited a significant delay in body weight loss and CMAP decline compared to vehicle treated mice.

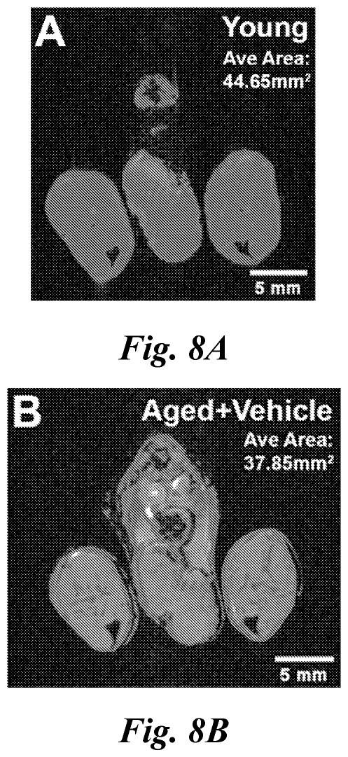

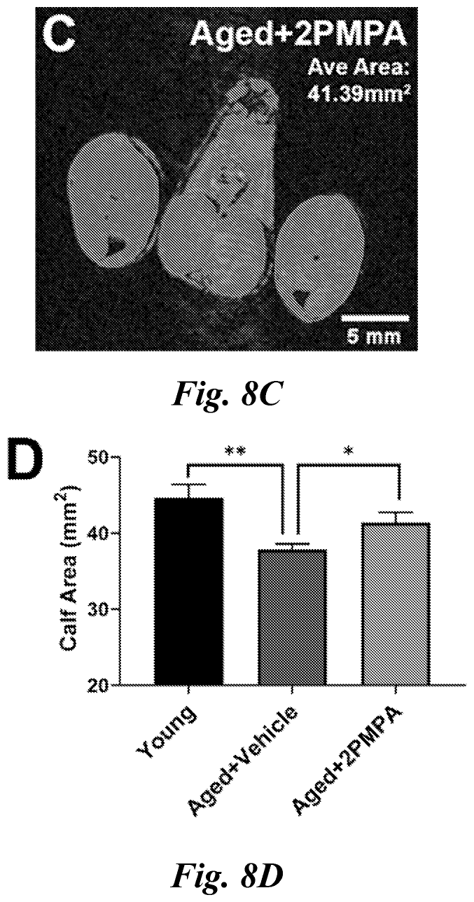

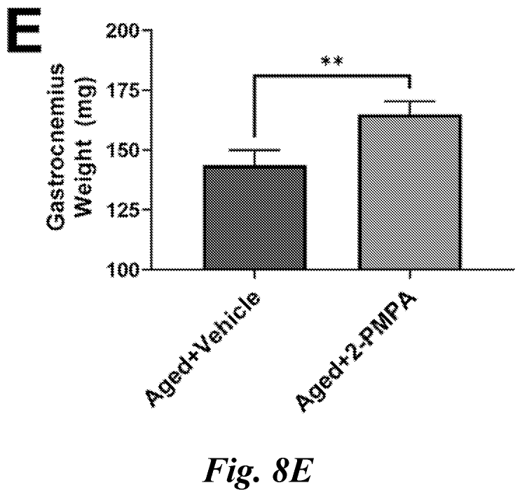

Further, FIG. 8A, FIG. 8B, FIG. 8C, FIG. 8D, and FIG. 8E demonstrates that muscle wasting is delayed in 2-PMPA treated aged animals. FIG. 8A, FIG. 8B, FIG. 8C, and FIG. 8D demonstrated that 8-weeks treatment of 2-PMPA improved whole calf muscle wasting as measured by MRI. FIG. 8E demonstrates that 20-weeks 2-PMPA treatment improved gastrocnemius muscle wasting as measured by dissected muscle weight (N=8-12/group);

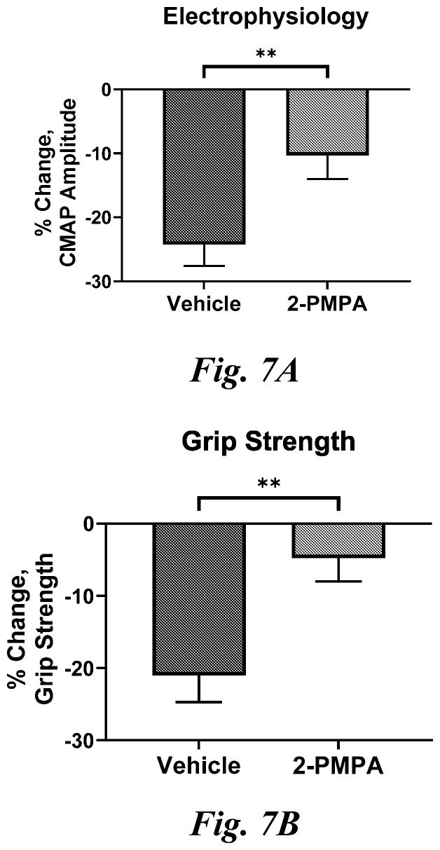

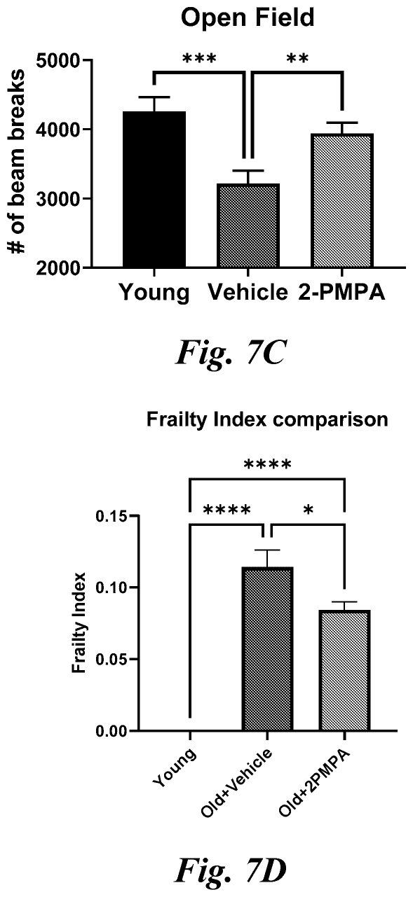

2-PMPA treatment also delayed CMAP amplitude and grip strength decline, with treated animals having significantly greater values after 5 months of treatment. 2-PMPA-treated animals also exhibited elevated ambulation in the open field test after 5 months of treatment. Referring now to FIG. 7A, FIG. 7B, and FIG. 7C, 2-PMPA treatment delays motor function loss in aged animals. FIG. 7A shows that CMAP decline is significantly reduced after 20-weeks treatment. FIG. 7B shows that hind limb grip strength decline is reduced after 20-weeks treatment. FIG. 7C shows that ambulatory movement is increased after 20-weeks treatment;

Further, monthly frailty scoring was performed using a frailty index that tracks a spectrum of aging-related characteristics. Referring now to FIG. 7D, after 5 months of treatment, 2-PMPA treated mice had significantly reduced frailty index scores compared with vehicle-treated mice. These studies demonstrate that blockade of GCPII activity has potential therapeutic benefits for slowing aging-related frailty.

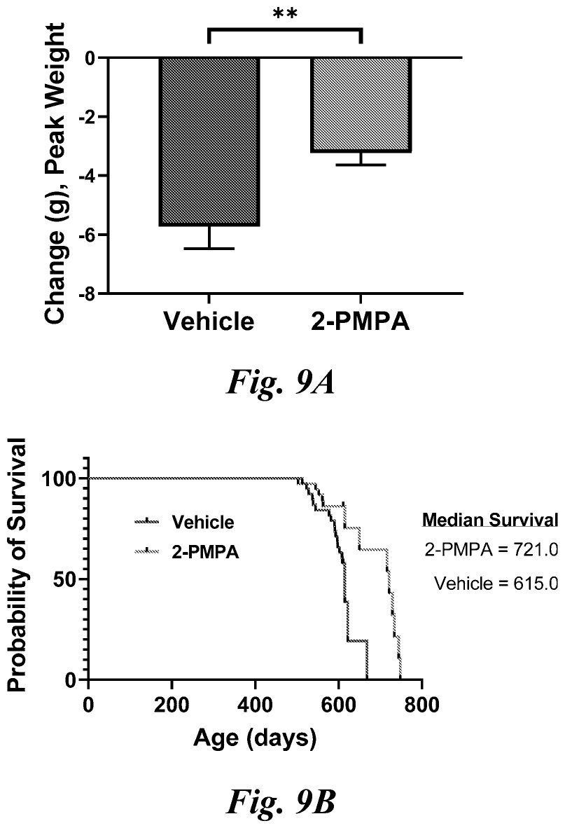

Unexpectantly, it also was found that chronic inhibition of GCPII can enhance longevity. 2-PMPA treatment significantly prolonged survival, with survival of 8% versus 58% of vehicle and 2-PMPA-treated mice, respectively, after 5 months of treatment. For example, the 2-PMPA treated animals survived significantly longer than the vehicle treated animals, with a median survival exceeding the vehicle mice by 106 days. FIG. 9A and FIG. 9B demonstrate that 2-PMPA treatment prolongs survival and delays weight loss. FIG. 9A demonstrates that 2-PMPA mice lost less body weight after 20 weeks on drug. FIG. 9B demonstrates that 2-PMPA treated mice survived longer than vehicle mice (N=48-50/group). These data support GCPII inhibition as a potential therapeutic strategy to delay muscle function loss related to aging and improve survival.

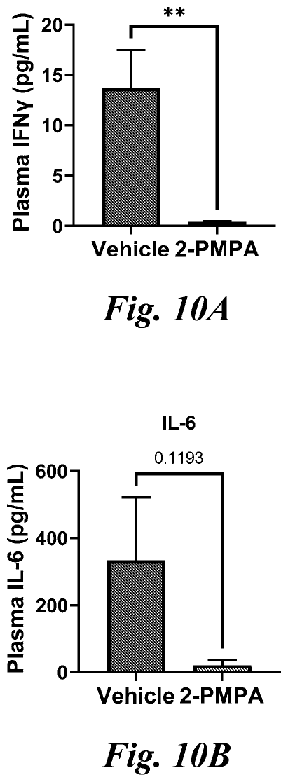

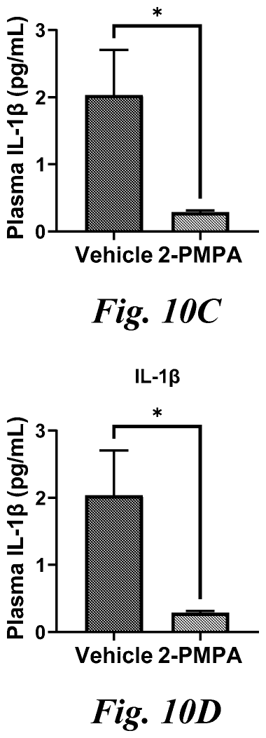

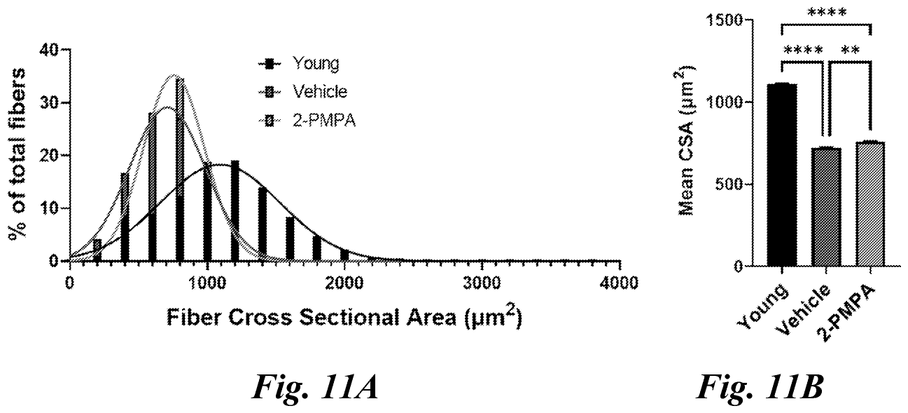

A striking reduction in several age-related proinflammatory cytokines in the plasma also was observed. More particularly, IFN-gamma, IL-1beta, and TNF-alpha were reduced. Referring now to FIG. 10A, FIG. 10B, FIG. 10C, and FIG. 10D, 20 weeks of 2-PMPA treatment reduces several circulating pro-inflammatory cytokines (N=8/group).

### 1.3 Summary

In summary, the presently disclosed data demonstrate that GCPII is elevated in aged WT mouse muscle and is associated with activated macrophages. GCPII inhibition delays motor functional loss and reduces muscle wasting. GCPII inhibition reduces weight loss and prolongs survival. GCPII inhibition reduces circulating pro-inflammatory cytokines.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents form part of the common general knowledge in the art.
Pandya, V. A. and Patani, R., Decoding the relationship between ageing and amyotrophic lateral sclerosis: a cellular perspective, Brain, Volume 143, Issue 4, April 2020, Pages 1057-1072.
Hu, Y., Cao, C., Qin, X Y. et al. Increased peripheral blood inflammatory cytokine levels in amyotrophic lateral sclerosis: a meta-analysis study. Sci Rep 7, 9094 (2017).
Pan L, Xie W, Fu X, Lu W, Jin H, Lai J, et al. Inflammation and sarcopenia: A focus on circulating inflammatory cytokines. Exp Gerontol. 2021; 154:111544.
Personius, K. E., Slusher, B. S., Udin, S. B., Neuromuscular NMDA Receptors Modulate Developmental Synapse Elimination, J. Neurosci. 24 Aug. 2016, 36 (34) 8783-8789.
Tallon, C., et al., Dendrimer-2PMPA Delays Muscle Function Loss and Denervation in a Murine Model of Amyotrophic Lateral Sclerosis, Neurotherapeutics, doi: 10.1007/s13311-021-01159-7 (2022).
Bařnka C., Rojas C., Slusher B., Pomper M., Glutamate carboxypeptidase II in diagnosis and treatment of neurologic disorders and prostate cancer. Curr Med Chem. 2012; 19(6):856-70.
Valdez G., Tapia J. C., Lichtman J. W., Fox M. A., Sanes J. R. (2012) Shared Resistance to Aging and ALS in Neuromuscular Junctions of Specific Muscles. PLoS ONE 7(4): e34640.
Vomov J. J., Peters D., Nedelcovych M., Hollinger K., Rais R., Slusher B. S. Looking for Drugs in All the Wrong Places: Use of GCPII Inhibitors Outside the Brain. Neurochem Res. 2020 June; 45(6):1256-1267.
Pastorino, S. et al. “Toward the Discovery and Development of PSMA Targeted Inhibitors for Nuclear Medicine Applications.” Current radiopharmaceuticals vol. 13, 1 (2020): 63-79.
Gourni E., Henriksen G., Metal-Based PSMA Radioligands. Molecules. 2017; 22(4):523.
Rais R, Vivra J, Tichf T, Dash R P, Gadiano A J, Tenora L, Monincová L, Bařnka C, Alt J, Zimmermann S C, Slusher C E, Wu Y, Wozniak K, Majer P, Tsukamoto T, Slusher B S. Discovery of a para-Acetoxy-benzyl Ester Prodrug of a Hydroxamate-Based Glutamate Carboxypeptidase II Inhibitor as Oral Therapy for Neuropathic Pain. J Med Chem. 2017 Sep. 28; 60(18):7799-7809.
U.S. Pat. No. 11,167,049, for Organ protection in PSMA-targeted radionuclide therapy of prostate cancer, to Babich et al., issued Nov. 9, 2021, which is incorporated herein by reference in its entirety.
Weineisen, M., Schottelius, M., Simecek, J., Baum, R. P., Yildiz, A., Beykan, S., Kulkarni, H. R., Lassmann, M., Klette, I., Eiber, M., Schwaiger, M., Wester, H.-J., 68Ga- and 177Lu-Labeled PSMA I&T: Optimization of a PSMA-Targeted Theranostic Concept and First Proof-of-Concept Human Studies, J. Nucl. Med. August 2015, 56 (8) 1169-1176.
Benešová M, Schafer M, Bauder-Wüst U, et al. Preclinical evaluation of a tailor-made DOTA-conjugated PSMA inhibitor with optimized linker moiety for imaging and endoradiotherapy of pr, ostate cancer. J Nucl Med. 2015; 56:914-920.
Eder M, Schafer M, Bauder-Wust U, Hull W E, Wangler C, Mier W, Haberkorn U, Eisenhut M. 68Ga-complex lipophilicity and the targeting property of a urea-based PSMA inhibitor for PET imaging. Bioconjug Chem. 2012; 23:688-697.
Chen, Y., Foss, C. A., Byun, Y., Nimmagadda, S., Pullambhatla, M., Fox, J. J., Castanares, M., Lupold, S. E., Babich, J. W., Mease, R. C., and Pomper, M. G., Radiohalogenated Prostate-Specific Membrane Antigen (PSMA)-Based Ureas as Imaging Agents for Prostate Cancer, J. Med. Chem. 2008, 51, 24, 7933-7943.
Knedlik T., Vorlová B., Navrátil V., Tykvart J., Sedlák F., Vaculin Š., Franěk M., Šácha P., Konvalinka J., Mouse glutamate carboxypeptidase II (GCPII) has a similar enzyme activity and inhibition profile but a different tissue distribution to human GCPII. FEBS Open Bio. 2017 Aug. 29; 7(9):1362-1378.
Chen Y., Pullambhatla M., Foss C. A., Byun Y., Nimmagadda S., Senthamizhchelvan S., Sgouros G., Mease R. C., Pomper M. G., 2-(3-{1-Carboxy-5-[(6-[18F]fluoro-pyridine3-carbonyl)-amino]-pentyl}-ureido)-pentanedioic acid, [18F]DCFPyL, a PSMA-based PET imaging agent for prostate cancer. Clin Cancer Res. 2011; 17:7645-53.
Wozniak, K. M., Wu, Y., Vornov, J. J., Lapidus, R., Rais, R., Rojas, C., Tsukamoto, T., and Slusher, B. S., The Orally Active Glutamate Carboxypeptidase II Inhibitor E2072 Exhibits Sustained Nerve Exposure and Attenuates Peripheral Neuropathy, Journal of Pharmacology and Experimental Therapeutics Dec. 1, 2012, 343 (3) 746-754.
Slusher, B. S., Thomas, A., Paul, M., Schad, C. A. and Ashby, C. R., Jr., Expression and acquisition of the conditioned place preference response to cocaine in rats is blocked by selective inhibitors of the enzyme N-acetylated-alpha-linked-acidic dipeptidase (NAALADase), Synapse (2001) 41, 22-28.
Jackson P. F., Slusher B. S., Design of NAALADase inhibitors: a novel neuroprotective strategy. Curr Med Chem. 2001; 8:949-957.
Ding P., Miller M. J., Chen Y., Helquist P., Oliver A. J., Wiest O. Syntheses of conformationally constricted molecules as potential NAALADase/PSMA inhibitors. Org. Lett. 2004; 6:1805-1808.
Ding P., Miller M. J., Chen Y., Helquist P., Oliver A. J., Wiest O., Syntheses of conformationally constricted molecules as potential NAALADase/PSMA inhibitors. Org. Lett. 2004; 6:1805-1808.
Bařnka C., Rojas C., Slusher B., Pomper M., Glutamate carboxypeptidase II in diagnosis and treatment of neurologic disorders and prostate cancer. Curr Med Chem. 2012; 19(6):856-870.

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A method for treating age-related sarcopenia and/or enhancing longevity, the method comprising administering to a subject in need of treatment thereof one or more GCPII inhibitors.

2. The method of claim 1, wherein the one or more GCPII inhibitors are selected from a phosphonate-based GCPII inhibitor, a phosphinate-based GCPII inhibitor, a phosphoramidate-based GCPII inhibitor, a thiol-based inhibitor, a hydroxamate-based inhibitor, and a urea-glutamate based GCPII inhibitor.

3. The method of claim 2, wherein;
(a) the phosphonate-based GCPII inhibitor is selected from 2-(phosphonomethyl) pentanedioic acid (2-PMPA), GPI-5232, and VA-033;
(b) the phosphinate-based GCPII inhibitor is selected from 2-[[methylhydroxyphosphinyl]methyl]pentanedioic acid, 2-[[ethylhydroxyphosphinyl]methyl]pentanedioic acid, 2-[[propylhydroxyphosphinyl]methyl]pentanedioic acid, 2-[[butylhydroxyphosphinyl]methyl]pentanedioic acid, 2-[[cyclohexylhydroxyphosphinyl]methyl]pentanedioic acid, 2-[[phenylhydroxyphosphinyl]methyl]pentanedioic acid, 2-[[(phenylmethyl)hydroxyphosphinyl]methyl]pentanedioic acid, 2-[[((2-phenylethyl)methyl)hydroxyphosphinyl]methyl]pentanedioic acid, 2-[[((3-phenylpropyl)methyl)hydroxyphosphinyl]methyl]pentanedioic acid, 2-[[((3-phenylbutyl)methyl)hydroxyphosphinyl]methyl]pentanedioic acid 2-[[((2-phenylbutyl)methyl)hydroxyphosphinyl]methyl]pentanedioic acid, 2-[[(4-phenylbutyl)hydroxyphosphinyl]methyl]pentanedioic acid, 2-[[(aminomethyl)hydroxyphosphinyl]methyl]pentanedioic acid; 7-(L-2-amino-2-carboxyethylthio)-2-(2,2-dimethylcyclopropanecarboxamide)-2-heptenoic acid, 2-(phosphonomethyl)pentanedioic acid, N-[methylhydroxyphosphinyl]glutamic acid; N-[ethylhydroxyphosphinyl]glutamic acid, N-[propylhydroxyphosphinyl]glutamic acid; N-[butylhydroxyphosphinyl]glutamic acid, N-[phenylhydroxyphosphinyl]glutamic acid; and N-[(phenylmethyl)hydroxyphosphinyl]glutamic acid;
(c) the thiol-based GCPII inhibitor is selected from 2-(3-mercaptopropyl)pentanedioic acid (2-MPPA), 3-(2-mercaptoethyl)biphenyl-2,3-dicarboxylic acid (E2072) and GPI-5693,
(d) the hydroxamate-based GCPII inhibitor is selected from 2-(2-(hydroxyamino)-2-oxoethyl)pentanedioic acid (JHU 241) and 4-carboxy-alpha-[3-(hydroxyamino)-3-oxopropyl]-benzenepropanoic acid; and
(e) the urea-glutamate based GCPII inhibitor is selected from N—[N—[(S)]-1,3-dicarboxypropyl]carbamoyl]-L-leucine (ZJ-43), MIP-1555, MIP-1519, MIP-1545, MIP-1427, MIP-1428, MIP-1379, MIP-1072, MIP-1095, MIP-1558, MIP-1405, MIP-1404, PSMA I&T, PSMA-617, PSMA-11, DCIBzL, ¹⁸F-DCFPyl, ZJ 38, GCPII-IN-1, and JB-352.

4-7. (canceled)

8. The method of claim 1, wherein the GCPII inhibitor is selected from quisqualate, β-citryl-L-glutamate, (S)-2-((((S)-5-(4-bromo-2-fluorobenzamido)-1-carboxypentyl)carbamoyl)oxy)pentanedioic acid; and (S)-2-((S)-1-carboxy-3-methylbutylcarbamoyloxy)pentanedioic acid.

9. The method of claim 1, wherein the GCPII inhibitor is selected from one or more of 2-PMPA and prodrugs thereof, L-DOPA, D-DOPA, caffeic acid, and prodrugs thereof, a hydroxamate-based prodrug, and a dendrimer 2-PMPA conjugate.

10. The method of claim 9, wherein the 2-PMPA and prodrugs thereof is a compound of formula (Ia) or formula (Ib): 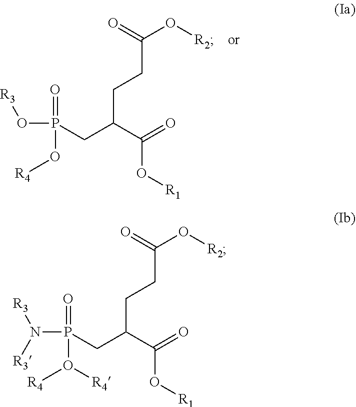
wherein:
each R₁, R₂, R₃, and R₄ is independently selected from the group consisting of H, alkyl, Ar, —(CR₅R₆)ₙ—Ar, —(CR₅R₆)ₙ—O—C(═O)—R₇, —(CR₅R₆)ₙ—C(═O)—O—R₇, —(CR₅R₆)ₙ—O—C(═O)—O—R₇, —(CR₅R₆)ₙ—O—R₇, —(CR₅R₆)ₙ—O—[(CR₅R₆)ₙ—O]ₘ—R₇, —(CR₅R₆)ₙ—Ar—O—C(═O)—R₇, —Ar—C(═O)—O—(CR₅R₆)ₙ—R₇, —(CR₅R₆)ₙ—NR₈R₉, and —(CR₅R₆)ₙ—C(═O)—NR₈R₉;
wherein:
n is an integer from 1 to 20;
m is an integer from 1 to 20;
each R₃′ and R₄′ are independently H or alkyl;
each R₅ and R₆ is independently selected from the group consisting of H, alkyl, and alkylaryl;
each R₇ is independently straight chain or branched alkyl;
Ar is aryl, substituted aryl, heteroaryl or substituted heteroaryl; and
R₈ and R₉ are each independently H or alkyl; and
pharmaceutically acceptable salts thereof.

11. The method of claim 10, wherein:
(a) each R₁ is H;
each R₂ is selected from the group consisting of H, alkyl, Ar, —(CR₅R₆)ₙ—Ar, —(CR₅R₆)ₙ—O—C(═O)—R₇, —(CR₅R₆)ₙ—C(═O)—O—R₇, —(CR₅R₆)ₙ—O—C(═O)—O—R₇, —(CR₅R₆)ₙ—O—R₇, —(CR₅R₆)ₙ—O—[(CR₅R₆)ₙ—O]ₘ—R₇, —(CR₅R₆)ₙ—Ar—O—C(═O)—R₇, —Ar—C(═O)—O—(CR₅R₆)ₙ—R₇, —(CR₅R₆)ₙ—NR₈R₉, and —(CR₅R₆)ₙ—C(═O)—NR₈R₉;
each R₃ is selected from the group consisting of H, alkyl, Ar, —(CR₅R₆)ₙ—Ar, —(CR₅R₆)ₙ—O—C(═O)—R₇, —(CR₅R₆)ₙ—C(═O)—O—R₇, —(CR₅R₆)ₙ—O—C(═O)—O—R₇, —(CR₅R₆)ₙ—O—R₇, —(CR₅R₆)ₙ—O—[(CR₅R₆)ₙ—O]ₘ—R₇, —(CR₅R₆)ₙ—Ar—O—C(═O)—R₇, —Ar—C(═O)—O—(CR₅R₆)ₙ—R₇, —(CR₅R₆)ₙ—NR₈R₉, and —(CR₅R₆)ₙ—C(═O)—NR₈R₉; and
each R₄ is selected from the group consisting of —(CR₅R₆)ₙ—O—C(═O)—R₇, —(CR₅R₆)ₙ—C(═O)—O—R₇, —(CR₅R₆)ₙ—O—C(═O)—O—R₇, —(CR₅R₆)ₙ—O—R₇, —(CR₅R₆)ₙ—O—[(CR₅R₆)ₙ—O]ₘ—R₇, —(CR₅R₆)ₙ—Ar—O—C(═O)—R₇, —Ar—C(═O)—O—(CR₅R₆)ₙ—R₇, —(CR₅R₆)ₙ—NR₈R₉, and —(CR₅R₆)ₙ—C(═O)—NR₈R₉;
(b) each R₁ is alkyl;
each R₂ is selected from the group consisting of H, alkyl, Ar, —(CR₅R₆)ₙ—Ar, —(CR₅R₆)ₙ—O—C(═O)—R₇, —(CR₅R₆)ₙ—C(═O)—O—R₇, —(CR₅R₆)ₙ—O—C(═O)—O—R₇, —(CR₅R₆)ₙ—O—R₇, —(CR₅R₆)ₙ—O—[(CR₅R₆)ₙ—O]ₘ—R₇, —(CR₅R₆)ₙ—Ar—O—C(═O)—R₇, —Ar—C(═O)—O—(CR₅R₆)ₙ—R₇, —(CR₅R₆)ₙ—NR₈R₉, and —(CR₅R₆)ₙ—C(═O)—NR₈R₉;
each R₃ is selected from the group consisting of H, alkyl, Ar, —(CR₅R₆)ₙ—Ar, —(CR₅R₆)ₙ—O—C(═O)—R₇, —(CR₅R₆)ₙ—C(═O)—O—R₇, —(CR₅R₆)ₙ—O—C(═O)—O—R₇, —(CR₅R₆)ₙ—O—R₇, —(CR₅R₆)ₙ—O—[(CR₅R₆)ₙ—O]ₘ—R₇, —(CR₅R₆)ₙ—Ar—O—C(═O)—R₇, —Ar—C(═O)—O—(CR₅R₆)ₙ—R₇, —(CR₅R₆)ₙ—NR₈R₉, and —(CR₅R₆)ₙ—C(═O)—NR₈R₉; and
each R₄ is selected from the group consisting of Ar, —(CR₅R₆)ₙ—O—C(═O)—R₇, —(CR₅R₆)ₙ—C(═O)—O—R₇, —(CR₅R₆)ₙ—O—C(═O)—O—R₇, —(CR₅R₆)ₙ—O—R₇, —(CR₅R₆)ₙ—O—[(CR₅R₆)ₙ—O]ₘ—R₇, —(CR₅R₆)ₙ—Ar—O—C(═O)—R₇, —Ar—C(═O)—O—(CR₅R₆)ₙ—R₇, —(CR₅R₆)ₙ—NR₈R₉, and —(CR₅R₆)ₙ—C(═O)—NR₈R₉;
(c) each R₁ is —(CR₅R₆)ₙ—Ar;
each R₂ is selected from the group consisting of H, alkyl, Ar, —(CR₅R₆)ₙ—Ar, —(CR₅R₆)ₙ—O—C(═O)—R₇, —(CR₅R₆)ₙ—C(═O)—O—R₇, —(CR₅R₆)ₙ—O—C(═O)—O—R₇, —(CR₅R₆)ₙ—O—R₇, —(CR₅R₆)ₙ—O—[(CR₅R₆)ₙ—O]ₘ—R₇, —(CR₅R₆)ₙ—Ar—O—C(═O)—R₇, —Ar—C(═O)—O—(CR₅R₆)ₙ—R₇, —(CR₅R₆)ₙ—NR₈R₉, and —(CR₅R₆)ₙ—C(═O)—NR₈R₉;
each R₃ is selected from the group consisting of H, alkyl, Ar, —(CR₅R₆)ₙ—Ar, —(CR₅R₆)ₙ—O—C(═O)—R₇, —(CR₅R₆)ₙ—C(═O)—O—R₇, —(CR₅R₆)ₙ—O—C(═O)—O—R₇, —(CR₅R₆)ₙ—O—R₇, —(CR₅R₆)ₙ—O—[(CR₅R₆)ₙ—O]ₘ—R₇, —(CR₅R₆)ₙ—Ar—O—C(═O)—R₇, —Ar—C(═O)—O—(CR₅R₆)ₙ—R₇, —(CR₅R₆)ₙ—NR₈R₉, and —(CR₅R₆)ₙ—C(═O)—NR₈R₉; and
each R₄ is selected from the group consisting of Ar, —(CR₅R₆)ₙ—O—C(═O)—R₇, —(CR₅R₆)ₙ—C(═O)—O—R₇, —(CR₅R₆)ₙ—O—C(═O)—O—R₇, —(CR₅R₆)ₙ—O—R₇, —(CR₅R₆)ₙ—O—[(CR₅R₆)ₙ—O]ₘ—R₇, —(CR₅R₆)ₙ—Ar—O—C(═O)—R₇, —Ar—C(═O)—O—(CR₅R₆)ₙ—R₇, —(CR₅R₆)ₙ—NR₈R₉, and —(CR₅R₆)ₙ—C(═O)—NR₈R₉; or
(d) each R₁ is selected from Ar, —(CR₅R₆)ₙ—O—C(═O)—R₇, —(CR₅R₆)ₙ—C(═O)—O—R₇, —(CR₅R₆)ₙ—O—C(═O)—O—R₇, —(CR₅R₆)ₙ—O—R₇, —(CR₅R₆)ₙ—O—[(CR₅R₆)ₙ—O]ₘ—R₇, —(CR₅R₆)ₙ—Ar—O—C(═O)—R₇, —Ar—C(═O)—O—(CR₅R₆)ₙ—R₇, —(CR₅R₆)ₙ—NR₈R₉, and —(CR₅R₆)ₙ—C(═O)—NR₈R₉;
each R₂ is selected from the group consisting of H, alkyl, Ar, —(CR₅R₆)ₙ—Ar, —(CR₅R₆)ₙ—O—C(═O)—R₇, —(CR₅R₆)ₙ—C(═O)—O—R₇, —(CR₅R₆)ₙ—O—C(═O)—O—R₇, —(CR₅R₆)ₙ—O—R₇, —(CR₅R₆)ₙ—O—[(CR₅R₆)ₙ—O]ₘ—R₇, —(CR₅R₆)ₙ—Ar—O—C(═O)—R₇, —Ar—C(═O)—O—(CR₅R₆)ₙ—R₇, —(CR₅R₆)ₙ—NR₈R₉, and —(CR₅R₆)ₙ—C(═O)—NR₈R₉;
each R₃ is selected from the group consisting of H, alkyl, Ar, —(CR₅R₆)ₙ—Ar, —(CR₅R₆)ₙ—O—C(═O)—R₇, —(CR₅R₆)ₙ—C(═O)—O—R₇, —(CR₅R₆)ₙ—O—C(═O)—O—R₇, —(CR₅R₆)ₙ—O—R₇, —(CR₅R₆)ₙ—O—[(CR₅R₆)ₙ—O]ₘ—R₇, —(CR₅R₆)ₙ—Ar—O—C(═O)—R₇, —Ar—C(═O)—O—(CR₅R₆)ₙ—R₇, —(CR₅R₆)ₙ—NR₈R₉, and —(CR₅R₆)ₙ—C(═O)—NR₈R₉; and
each R₄ is selected from the group consisting of H, alkyl, Ar, —(CR₅R₆)ₙ—Ar, —(CR₅R₆)ₙ—O—C(═O)—R₇, —(CR₅R₆)ₙ—C(═O)—O—R₇, —(CR₅R₆)ₙ—O—C(═O)—O—R₇, —(CR₅R₆)ₙ—O—R₇, —(CR₅R₆)ₙ—O—[(CR₅R₆)ₙ—O]ₘ—R₇, —(CR₅R₆)ₙ—Ar—O—C(═O)—R₇, —Ar—C(═O)—O—(CR₅R₆)ₙ—R₇, —(CR₅R₆)ₙ—NR₈R₉, and —(CR₅R₆)ₙ—C(═O)—NR₈R₉;
wherein:
each n is an integer from 1 to 20;
each m is an integer from 1 to 20;
each R₅ and R₆ is independently selected from the group consisting of H, alkyl, and alkylaryl;
each R₇ is independently straight chain or branched alkyl;
each Ar is aryl, substituted aryl, heteroaryl or substituted heteroaryl;
each R₈ and R₉ are independently H or alkyl; and
each R₃′ and R₄′ are independently H or alkyl; and
pharmaceutically acceptable salts thereof.

12. The method of claim 11, wherein the compound is a compound of formula (Ia) and:
(a) R₁ is H;
R₂ and R₃ are each selected from the group consisting of H, —(CR₅R₆)ₙ—O—R₇, —(CR₅R₆)ₙ—Ar—O—C(═O)—R₇, —(CR₅R₆)ₙ—O—C(═O)—R₇, —Ar—C(═O)—O—(CR₅R₆)ₙ—R₇, and —(CR₅R₆)ₙ—O—C(═O)—O—R₇; and
R₄ is selected from the group consisting of —(CR₅R₆)ₙ—O—R₇, —(CR₅R₆)ₙ—Ar—O—C(═O)—R₇, —Ar—C(═O)—O—(CR₅R₆)ₙ—R₇, —(CR₅R₆)ₙ—O—C(═O)—R₇ and —(CR₅R₆)ₙ—O—C(═O)—O—R₇;
(b) R₁ is alkyl;
R₂ and R₃ are each independently selected from the group consisting of H, alkyl, —(CR₅R₆)ₙ—O—R₇—(CR₅R₆)ₙ—Ar—O—C(═O)—R₇, —(CR₅R₆)ₙ—O—[(CR₅R₆)ₙ—O]ₘ—R₇, —(CR₅R₆)ₙ—O—C(═O)—R₇ and —(CR₅R₆)ₙ—O—C(═O)—O—R₇; and
R₄ is selected from the group consisting of —(CR₅R₆)ₙ—O—R₇, —(CR₅R₆)ₙ—Ar—O—C(═O)—R₇, —(CR₅R₆)ₙ—O—[(CR₅R₆)ₙ—O]ₘ—R₇, —(CR₅R₆)ₙ—O—C(═O)—R₇ and —(CR₅R₆)ₙ—O—C(═O)—O—R₇;
(c) R₁ is selected from —(CR₅R₆)ₙ—O—C(═O)—R₇ and —(CR₅R₆)ₙ—O—C(═O)—O—R₇; and
R₂ R₃, and R₄ are each independently selected from H, Ar, —(CR₅R₆)ₙ—O—C(═O)—R₇, and —(CR₅R₆)ₙ—O—C(═O)—O—R₇;
(d) one of R₁, R₂, R₃, or R₄ is H and the other three are each independently selected from the group consisting of:
—(CR₅R₆)ₙ—O—C(═O)—R₇ and —(CR₅R₆)ₙ—O—C(═O)—O—R₇;
wherein R₅ and R₆ are each independently selected from the group consisting of H, C₁₋₈ straight-chain alkyl, and C₁₋₈ branched-chain alkyl;
R₇ is C₁₋₈ straight-chain alkyl, and C₁₋₈ branched-chain alkyl; or
(e) R₂ is H; and
R₁, R₃, and R₄ are each independently selected from the group consisting of:
—(CR₅R₆)ₙ—O—C(═O)—R₇ and —(CR₅R₆)ₙ—O—C(═O)—O—R₇;
wherein R₅ and R₆ are each independently selected from the group consisting of H, C₁₋₈ straight-chain alkyl, and C₁₋₈ branched-chain alkyl;
R₇ is C₁₋₈ straight-chain alkyl or C₁₋₈ branched-chain alkyl; and
pharmaceutically acceptable salts thereof.

13-16. (canceled)

17. The method of claim 11, wherein the compound of formula (Ia) or formula (Ib) is selected from the group consisting of:

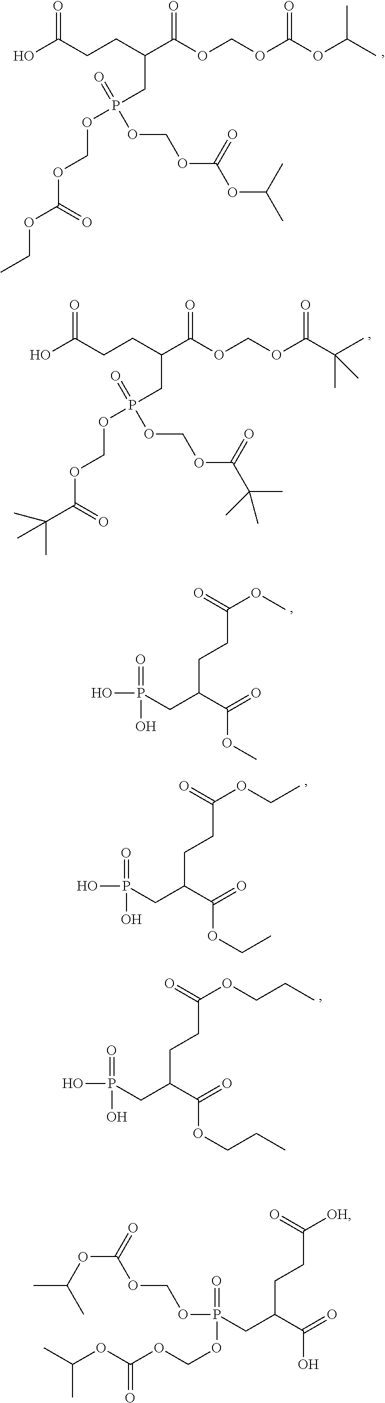

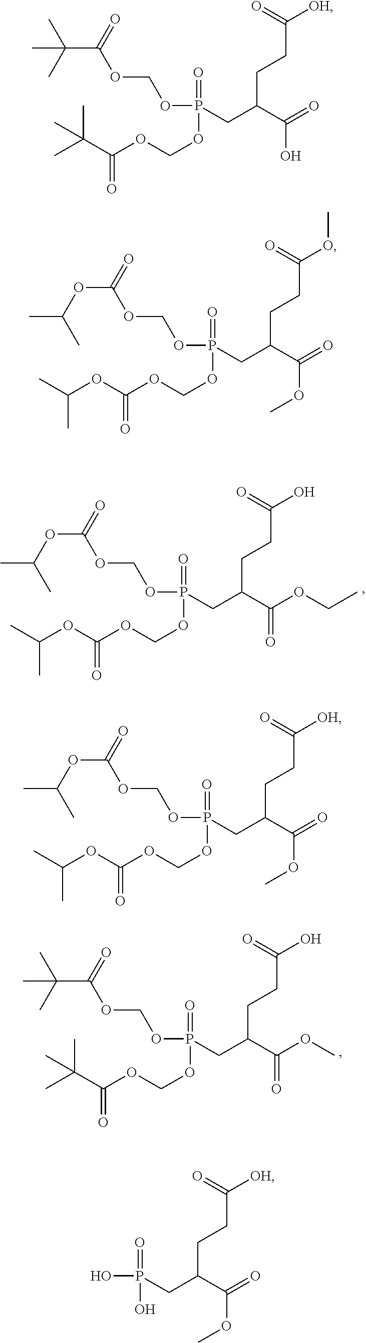

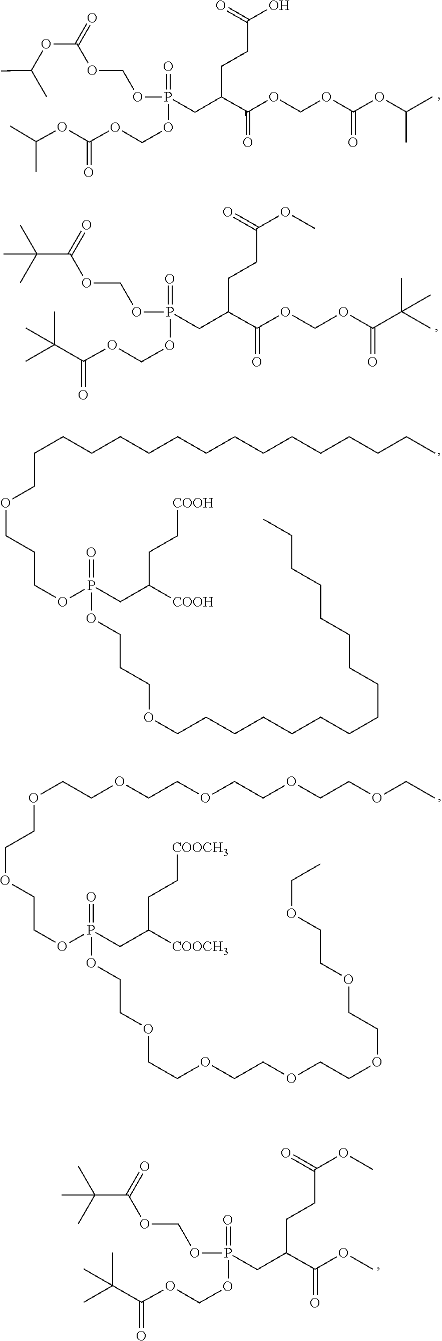

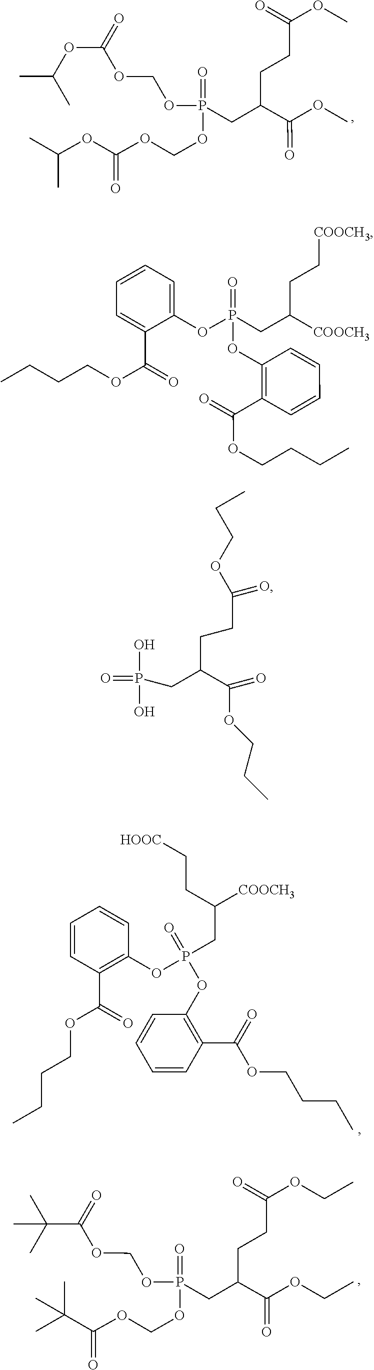

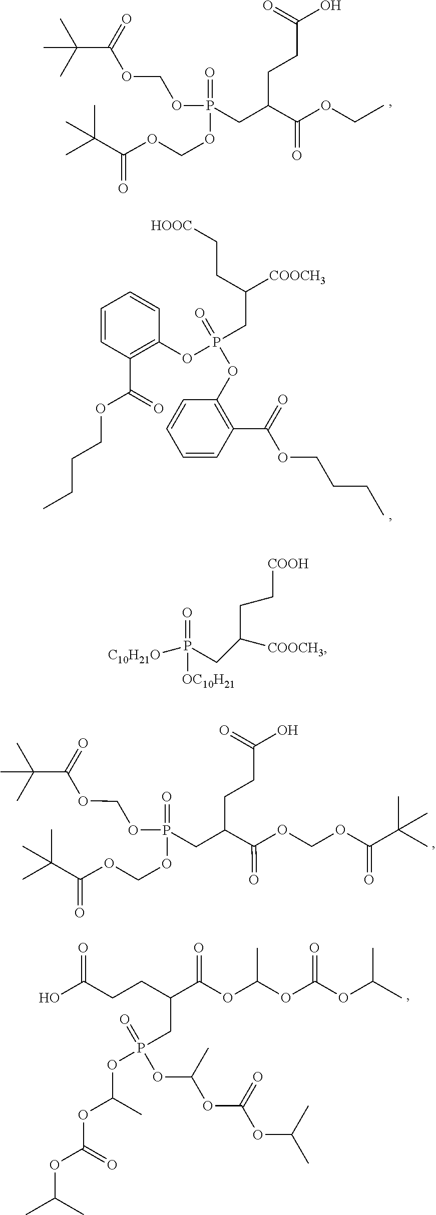

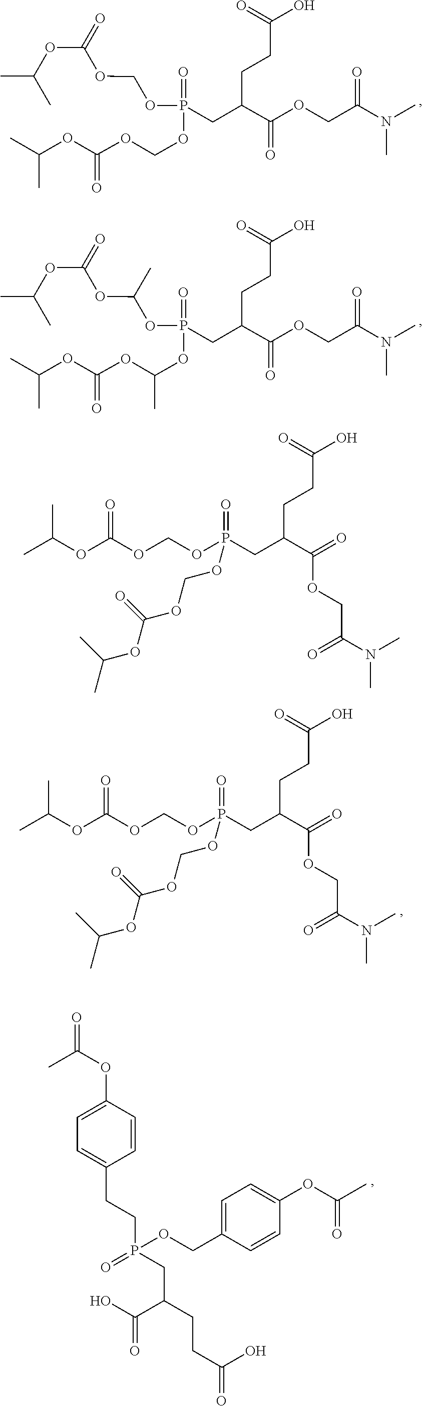

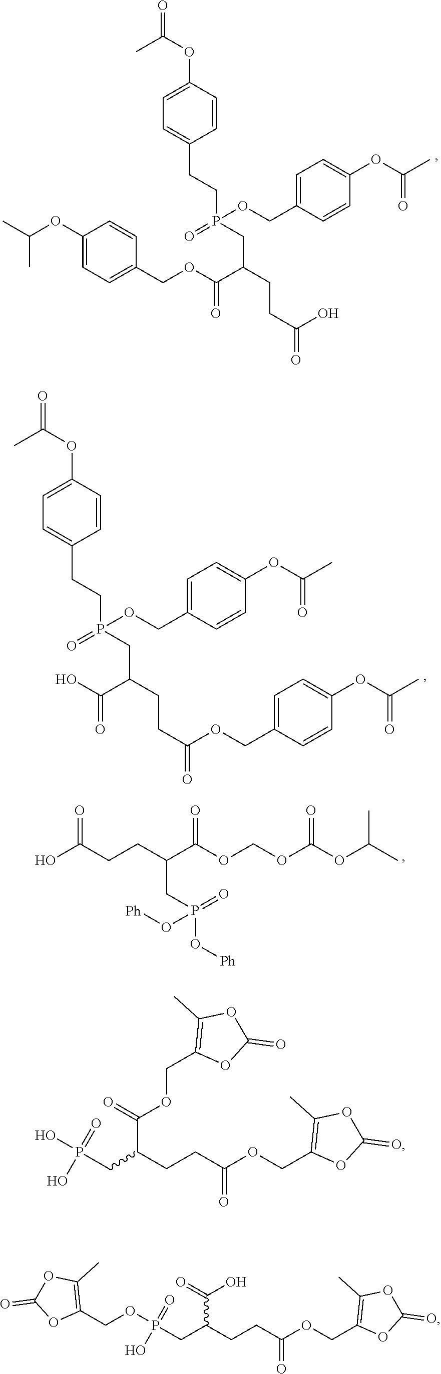

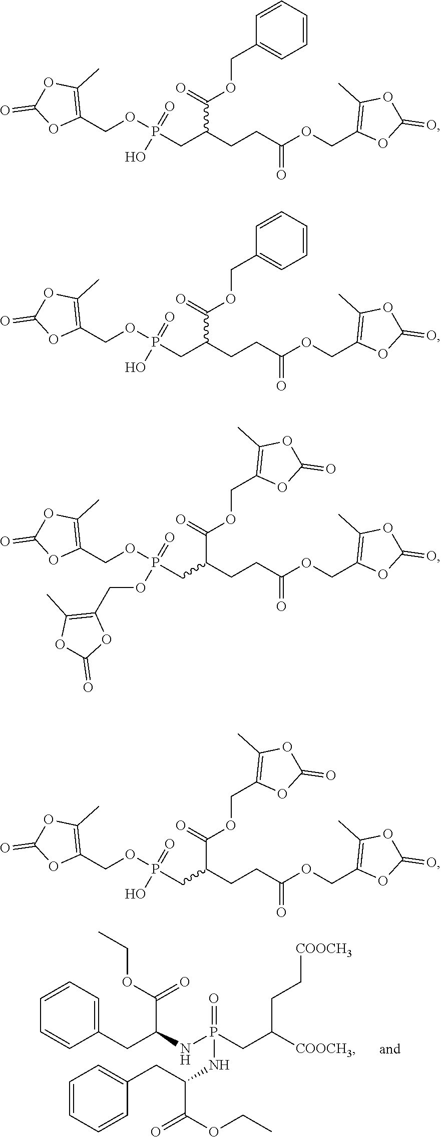

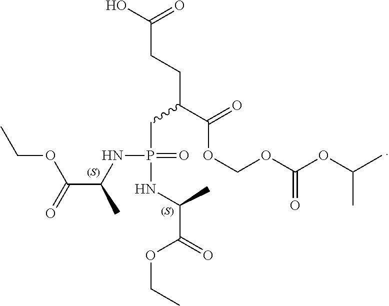

18. The method of claim 9, wherein the L-DOPA, D-DOPA, caffeic acid, and prodrugs thereof comprise a compound of formula (II): 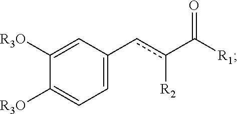
wherein: [symbol]
indicates that the bond can be a single or a double bond;
R₁ is:
—OR₅, wherein R₅ is selected from the group consisting of H, C₁-C₈ alkyl, and —O—(CH₂)ₙ—R₆, wherein n is an integer selected from the group consisting of 0, 1, 2, 3, 4, 5, 6, 7, and 8 and R₆ is substituted or unsubstituted aryl or heteroaryl; or
—NR₇R₈, wherein R₇ and R₈ are each independently selected from the group consisting of H, C₁-C₄ alkyl, C₃-C₆ cycloalkyl, C₁-C₈ alkoxyl, unsubstituted or substituted aryl or heteroaryl, —(CH₂)ₘ—R₉, wherein R₉ is —OR₁₀ or CHX₂, wherein R₁₀ is H or C₁-C₄ alkyl, and each X is halogen, and —(CH₂)ₘ—CH(NH₂)(COOH), wherein each m is independently an integer selected from the group consisting of 0, 1, 2, 3, 4, 5, 6, 7, and 8;
R₂ is H or —NR₁₁R₁₂, wherein R₁₁ and R₁₂ are each independently selected from the group consisting of H, C₁-C₄ alkyl, and —C(═O)—R₁₃, wherein R₁₃ is C₁-C₄ alkyl or —C(NH₂)—(CH₂)ₚ—R₁₄, wherein R₁₄ is C₁-C₄ alkyl or —NR₁₅R₁₆, wherein R₁₅ and R₁₆ are each H or C₁-C₄ alkyl, and p is an integer selected from the group consisting of 0, 1, 2, 3, 4, 5, 6, 7, and 8;
R₃ and R₄ are each independently H or —C(═O)—R₁₇, wherein R₁₇ is C₁-C₈ alkyl or —(CH₂)ₜ—O—C(═O)—O—R₁₈, wherein R₁₈ is C₁-C₈ alkyl, and t is an integer selected from the group consisting of 0, 1, 2, 3, 4, 5, 6, 7, and 8; and
stereoisomers and pharmaceutically acceptable salts thereof.

19. The method of claim 18, wherein:
(a) R₁ is —OR₅, and R₅ is selected from the group consisting of H, methyl, ethyl, n-propyl, isopropyl, n-butyl, isobutyl, sec-butyl, tert-butyl, n-pentyl, sec-pentyl, isopentyl, neopentyl, n-hexyl, sec-hexyl, n-heptyl, and n-octyl;
(b) R₁ is —OR₅, and R₅ is H or —O—(CH₂)ₙ—R₆, wherein R₆ is substituted or unsubstituted phenyl;
(c) R₁ is —NR₇R₈, and R₇ is H or C₁-C₄ alkyl and R₈ is selected from the group consisting of H, C₁-C₄ alkyl, C₃-C₆ cycloalkyl, unsubstituted or substituted phenyl, —(CH₂)ₘ—R₉, wherein R₉ is —OR₁₀ or CHX₂, wherein R₁₀ is H or C₁-C₄ alkyl, and each X is halogen, and —(CH₂)ₘ—CH(NH₂)(COOH), wherein each m is independently an integer selected from the group consisting of 0, 1, 2, 3, 4, 5, 6, 7, and 8;
(d) R₂ is —NR₁₁R₁₂, wherein R₁₁ is H and R₁₂ is H or —C(═O)—R₁₃, wherein R₁₃ is C₁-C₄ alkyl or —C(NH₂)—(CH₂)₁—R₁₄, wherein R₁₄ is C₁-C₄ alkyl or —NR₁₅R₁₆, wherein R₁₅ and R₁₆ are each H or C₁-C₄ alkyl, and p is an integer selected from the group consisting of 0, 1, 2, 3, 4, 5, 6, 7, and 8;
(e) if R₁ is —OR₅, then R₅ cannot be H; or if R₁ is —OR₅, then R₃, R₄, and R₅ cannot all be H;
(f) R₃ and R₄ are each independently selected from the group consisting of —C(═O)—CH₃, —C(═O)—C(CH₃)₃, and —CH₂—O—C(═O)—O—CH(CH₃)₂; or
(g) R₃ and R₄ are each H.

20-22. (canceled)

23. The method claim 18, wherein the compound of formula (II) is selected from the group consisting of:

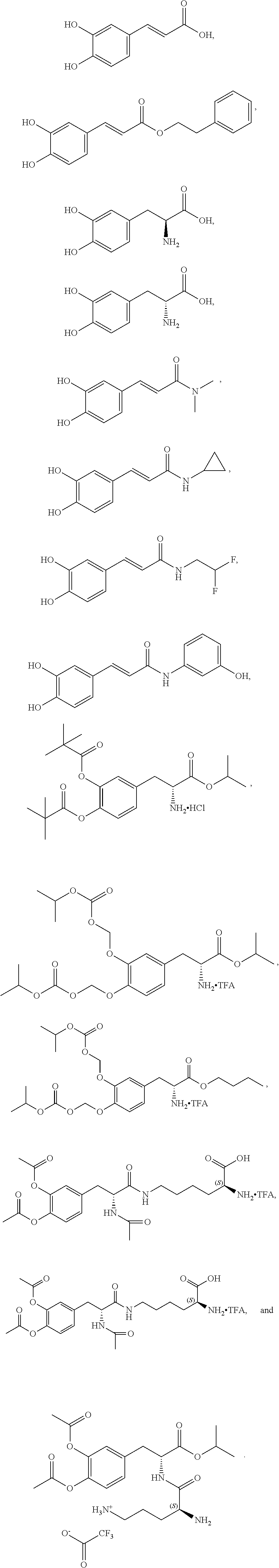

24. The method of claim 9, wherein the hydroxamate-based prodrug comprises a compound of formula (III): 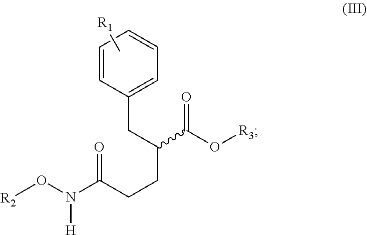
wherein:
R₁ is selected from the group consisting of —C(═O)—O—R₄ and —Ar—C(═O)—O—R₄;
R₂ is selected from the group consisting of substituted and unsubstituted C₁-C₆ alkyl, substituted and unsubstituted C₃-C₈ cycloalkyl, substituted and unsubstituted C₆-C₁₂ aryl, substituted and unsubstituted C₆-C₁₂ heteroaryl, —(CR₅R₆)ₙ—R₇, —C(═O)—O—R₇, —C(═O)—R₇, —C(═O)—NR₇R₈, —(CR₅R₆)ₙ—O—C(═O)—O—R₇, —(CR₅R₆)ₙ—Ar—O—C(═O)—R₇; R₃ is selected from the group consisting of H, substituted and unsubstituted C₁-C₆ alkyl, substituted and unsubstituted C₃-C₁₂ cycloalkyl, substituted and unsubstituted C₆-C₁₂ aryl, substituted and unsubstituted C₅-C₁₂ heteroaryl; R₄ is selected from the group consisting of H, substituted and unsubstituted C₁-C₆ alkyl, substituted and unsubstituted C₃-C₁₂ cycloalkyl, substituted and unsubstituted C₆-C₁₂ aryl, substituted and unsubstituted C₅-C₁₂ heteroaryl, —(CR₅R₆)ₙ—O—C(═O)—O—R₉, and —(CR₅R₆)ₙ—Ar—O—C(═O)—R₉; each R₅ and R₆ is independently selected from the group consisting of H, C₁-C₁₀ alkyl, and C₆-C₁₂ aralkyl; R₇ is selected from the group consisting of H, and substituted and unsubstituted C₁-C₁₀ alkyl, substituted and unsubstituted C₁-C₁₀ heteroalkyl, substituted and unsubstituted C₃-C₁₆ cycloalkyl, substituted and unsubstituted C₃-C₁₂ cycloheteroalkyl, substituted and unsubstituted C₃-C₁₂ cycloheteroalkenyl, substituted and unsubstituted C₆-C₁₂ aryl, substituted and unsubstituted C₆-C₁₂ heteroaryl, and substituted and unsubstituted C₆-C₁₂ aralkyl; R₈ is selected from the group consisting of H, and substituted and unsubstituted C₁-C₆ alkyl; R₉ is selected from the group consisting of H, and substituted and unsubstituted C₁-C₆ alkyl; n is an integer selected from the group consisting of 0, 1, 2, 3, 4, 5, and 6; Ar is selected from the group consisting of substituted and unsubstituted C₆-C₁₂ aryl, and substituted and unsubstituted C₆-C₁₂ heteroaryl; and stereoisomers and pharmaceutically acceptable salts thereof.

25. The method of claim 24, wherein the compound of formula (III) is selected from the group consisting of: 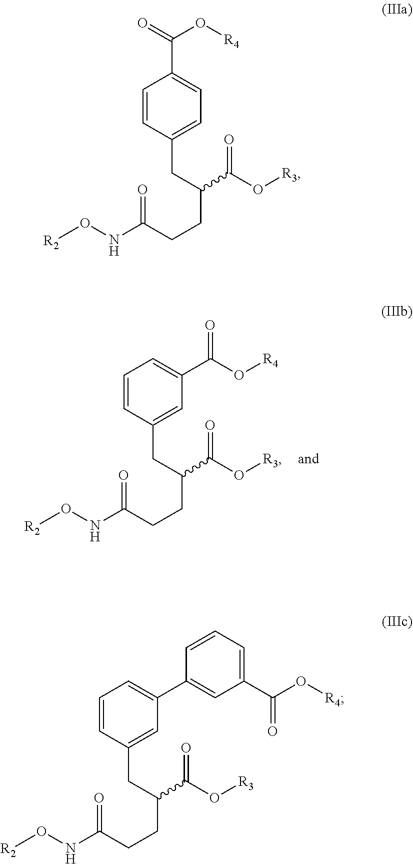
and stereoisomers and pharmaceutically acceptable salts thereof.

26. The method of claim 24, wherein:
(a) R₃ is selected from the group consisting of H and substituted and unsubstituted C₁-C₆ alkyl; R₄ is selected from the group consisting of H, substituted and unsubstituted C₁-C₆ alkyl, —(CR₅R₆)ₙ—Ar—O—C(═O)—R₉, and —(CR₅R₆)ₙ—O—C(═O)—O—R₉; n is 1; R₅ and R₆ are H; Ar is phenyl; R₉ is selected from the group consisting of substituted C₁-C₃ alkyl, and unsubstituted C₁-C₃ alkyl; and/or
(b) R₂ is —(CR₅R₆)ₙ—Ar—O—C(═O)—R₇, n is 1, Ar is phenyl, and R₇ is substituted or unsubstituted C₁-C₆ alkyl and stereoisomers and pharmaceutically acceptable salts thereof.

27. (canceled)

28. The method of claim 24, wherein the compound of formula (III) is selected from the group consisting of:

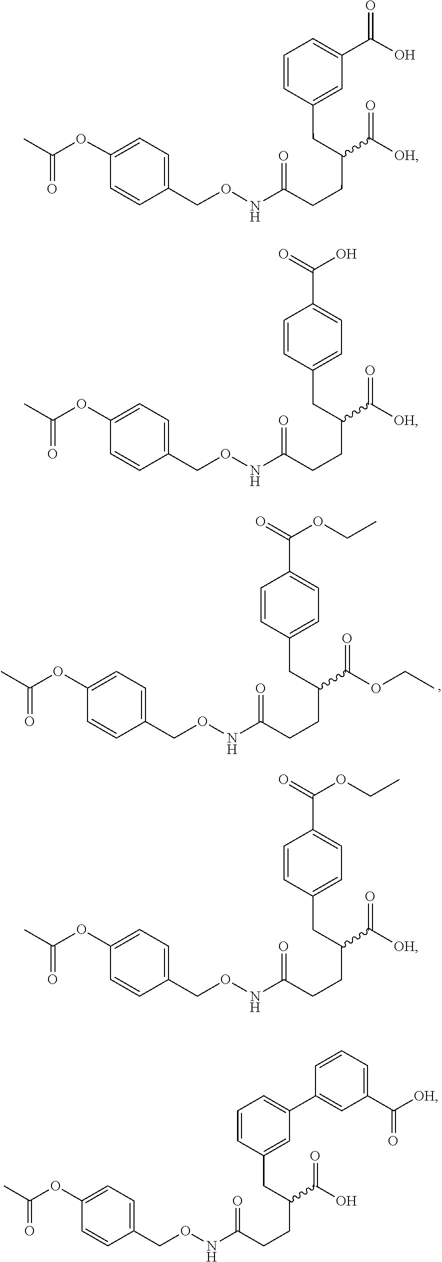

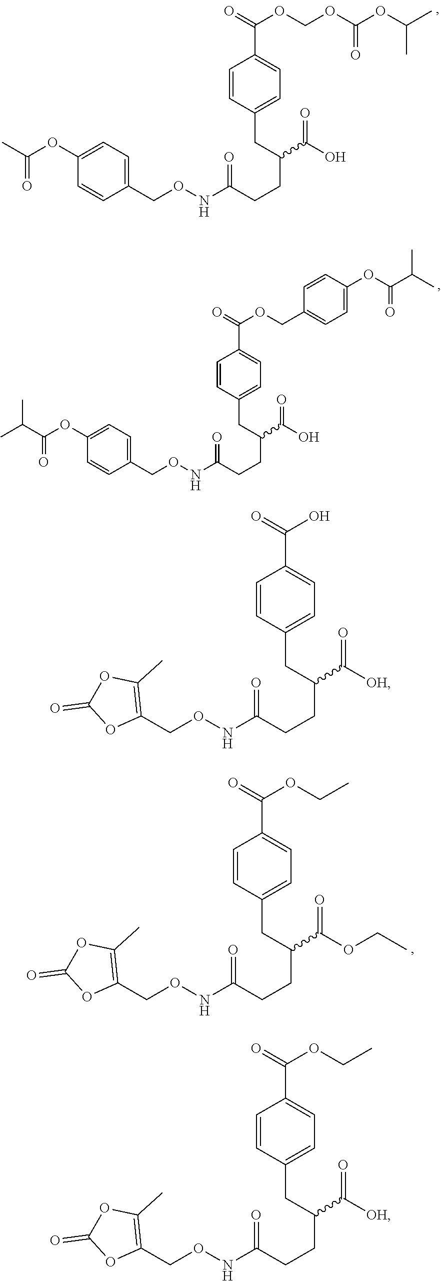

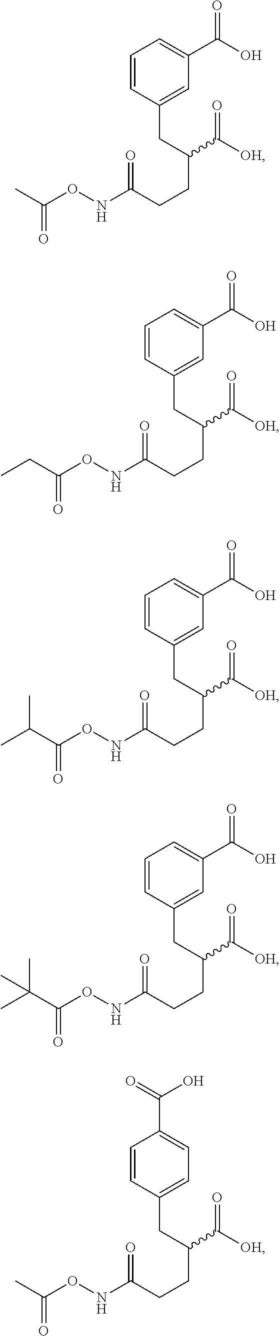 [SMILES: CC(=O)ONC(=O)CCC(Cc1cccc(C(=O)O)c1)C(=O)O ; CCC(=O)ONC(=O)CCC(Cc1cccc(C(=O)O)c1)C(=O)O ; CC(C)C(=O)ONC(=O)CCC(Cc1cccc(C(=O)O)c1)C(=O)O ; CC(C)(C)C(=O)ONC(=O)CCC(Cc1cccc(C(=O)O)c1)C(=O)O ; CC(=O)ONC(=O)CCC(Cc1ccc(C(=O)O)cc1)C(=O)O]

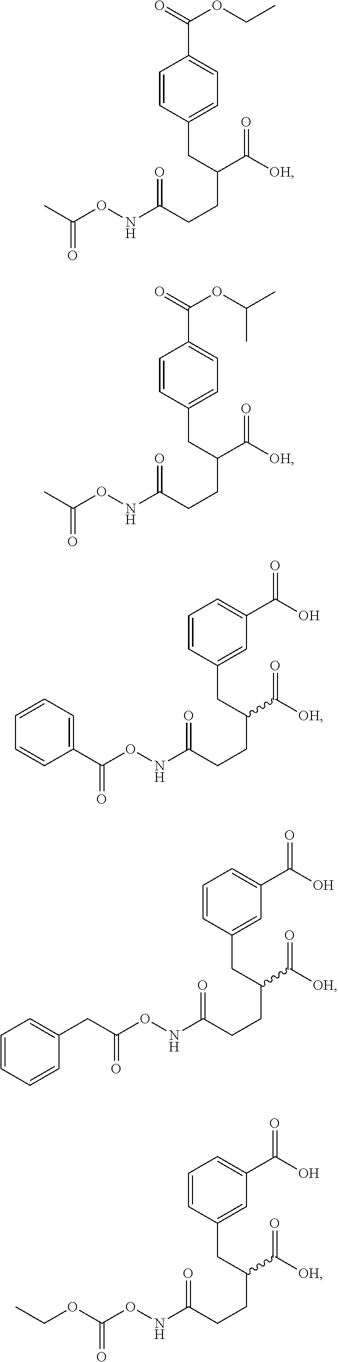

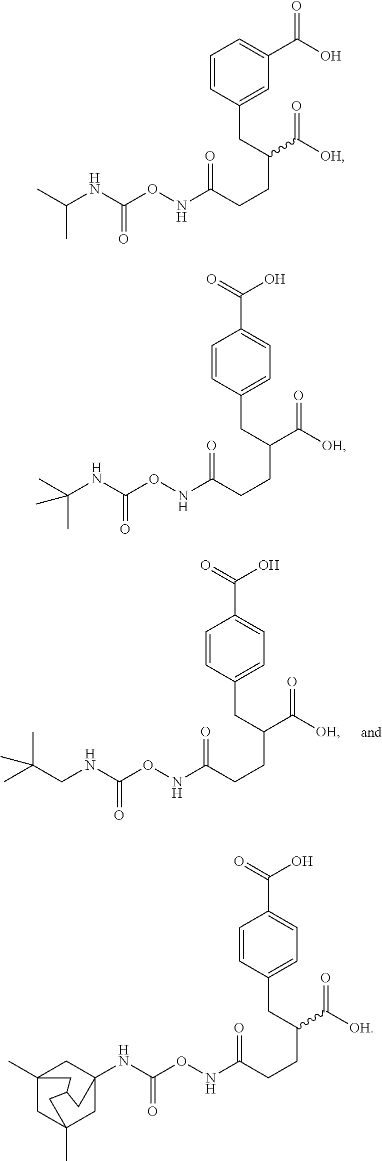 [SMILES: CC(C)NC(=O)ONC(=O)CCC(Cc1cccc(C(=O)O)c1)C(=O)O ; CC(C)(C)NC(=O)ONC(=O)CCC(Cc1ccc(C(=O)O)cc1)C(=O)O ; CC(C)(C)CNC(=O)ONC(=O)CCC(Cc1ccc(C(=O)O)cc1)C(=O)O ; "and" ; CC12CC3CC(C)(C1)CC(NC(=O)ONC(=O)CCC(Cc1ccc(C(=O)O)cc1)C(=O)O)(C3)C2]

29. The method of claim 9, wherein the dendrimer 2-PMPA conjugate comprises one or more dendrimers selected from the group consisting of polyamidoamine (PAMAM), polypropyiamine (POPAM), poly(propylene imine) (PPI), polyethylenimine, polylysine, polyester, iptycene, aliphatic poly(ether), aromatic polyether dendrimers, and combinations thereof.

30. The method of claim 29, wherein the dendrimer comprises a generation-4 (G4) to generation-10 (G10) PAMAM dendrimer having a terminal group selected from the group consisting of a carboxylic group, an amine group, and a hydroxyl group.

31. The method of claim 29, wherein the dendrimer 2-PMPA conjugate comprises a generation-4 through generation-10 hydroxyl-terminated PAMAM dendrimer covalently linked to 2-PMPA.

32. The method of claim 31, wherein the PAMAM dendrimer is covalently linked to 2-PMPA through a disulfide bridge.
